SEQUENCE LISTING (179 sequences)

SEQ ID NO: 1 - protein, 237 aa, Artificial Sequence
  Synthetic sequence
  MISC_FEATURE (1)..(23): Signal peptide of MIP1-alpha
  MISC_FEATURE (24)..(93): Amino acid sequence of human MIP1-alpha
  MISC_FEATURE (94)..(105): hinge exon h1 from IgG3
  MISC_FEATURE (106)..(120): hinge exon h4 from IgG3
  MISC_FEATURE (121)..(130): exemplary dimerization unit linker
  MISC_FEATURE (131)..(237): human CH3 domain of IgG3
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 2 - protein, 260 aa, Artificial Sequence
  Synthetic sequence
  MISC_FEATURE (1)..(19): Amino acid sequence of anti-pan HLA class II with Ig VH signal peptide
  MISC_FEATURE (20)..(127): anti-pan HLA class II VL
  MISC_FEATURE (128)..(142): exemplary linker
  MISC_FEATURE (143)..(260): anti-pan HLA class II VH
MNFGLRLIFLVLTLKGVQCDIQMTQTTSSLSASLGDRVTISCSASQDINNYLNWYQQKPDGTVKLLIYYTSSLHSGVPSRFSGSGSGTDYSLTISNLEPEDIATYYCQQYSKFPRTFGGGTKLEIKRGGGGSGGGGSGGGGSQIQLVQSGPELKKPGETVKISCKASGYTFINYGMNWVKQTPGKGLKWMGWINTYSGEPTYPDDFKGRFAFSLETSASTAYLQLNNLKNEDMATYFCARGDYYGPFDNWGQGTTLTVSS

SEQ ID NO: 3 - protein, 22 aa, Artificial Sequence
  Synthetic sequence
  MISC_FEATURE (1)..(22): Signal peptide
MDAMKRGLCCVLLLCGAVFVSP

SEQ ID NO: 4 - protein, 500 aa, Artificial Sequence
  Synthetic sequence
  MISC_FEATURE (1)..(500): VB2081
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 5 - protein, 491 aa, Artificial Sequence
  Synthetic sequence
  MISC_FEATURE (1)..(491): VB2082
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLFVNEFYAYLRKHFSMMGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 6 - protein, 526 aa, Artificial Sequence
  Synthetic sequence
  MISC_FEATURE (1)..(526): VB2083
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 7 - protein, 551 aa, Artificial Sequence
  Synthetic sequence
  MISC_FEATURE (1)..(551): VB2084
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 8 - protein, 495 aa, Artificial Sequence
  Synthetic sequence
  MISC_FEATURE (1)..(495): VB2085
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 9 - protein, 500 aa, Artificial Sequence
  Synthetic sequence
  MISC_FEATURE (1)..(500): VB2086
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGLGGLGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 10 - protein, 486 aa, Artificial Sequence
  Synthetic sequence
  MISC_FEATURE (1)..(486): VB2087
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLFVNEFYAYLRKHFSMMGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 11 - protein, 521 aa, Artificial Sequence
  Synthetic sequence
  MISC_FEATURE (1)..(521): VB2088
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 12 - protein, 546 aa, Artificial Sequence
  Synthetic sequence
  MISC_FEATURE (1)..(546): VB2089
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 13 - protein, 506 aa, Artificial Sequence
  Synthetic sequence
  MISC_FEATURE (1)..(506): VB2091
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 14 - protein, 557 aa, Artificial Sequence
  Synthetic sequence
  MISC_FEATURE (1)..(557): VB2092
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 15 - protein, 503 aa, Artificial Sequence
  Synthetic sequence
  MISC_FEATURE (1)..(503): VB2094
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 16 - protein, 554 aa, Artificial Sequence
  Synthetic sequence
  MISC_FEATURE (1)..(554): VB2095
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 17 - protein, 577 aa, Artificial Sequence
  Synthetic sequence
  MISC_FEATURE (1)..(577): VB2097
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLGGSAGGSGSGSSGGSSGASGTGTAGGTGSGSGTGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 18 - protein, 577 aa, Artificial Sequence
  Synthetic sequence
  MISC_FEATURE (1)..(577): VB2099
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLGGSGGGSEGGGSEGGGSEGGGSEGGGSEGGGSGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 19 - protein, 10 aa, Artificial Sequence
  Synthetic sequence
  MISC_FEATURE (1)..(10): Linker
GGGGSGGGSS

SEQ ID NO: 20 - protein, 5 aa, Artificial Sequence
  Synthetic sequence
  MISC_FEATURE (1)..(5): Linker
GGGSG

SEQ ID NO: 21 - protein, 5 aa, Artificial Sequence
  Synthetic sequence
  MISC_FEATURE (1)..(5): Linker
GGGGS

SEQ ID NO: 22 - protein, 5 aa, Artificial Sequence
  Synthetic sequence
  MISC_FEATURE (1)..(5): Linker
GGGGS

SEQ ID NO: 23 - protein, 5 aa, Artificial Sequence
  Synthetic sequence
  MISC_FEATURE (1)..(5): Linker
GGGSS

SEQ ID NO: 24 - protein, 5 aa, Artificial Sequence
  Synthetic sequence
  MISC_FEATURE (1)..(5): Linker
GGGSG

SEQ ID NO: 25 - protein, 5 aa, Artificial Sequence
  Synthetic sequence
  MISC_FEATURE (1)..(5): Linker
LGGGS

SEQ ID NO: 26 - protein, 5 aa, Artificial Sequence
  Synthetic sequence
  MISC_FEATURE (1)..(5): Linker
GLGGS

SEQ ID NO: 27 - protein, 5 aa, Artificial Sequence
  Synthetic sequence
  MISC_FEATURE (1)..(5): Linker
GGLGS

SEQ ID NO: 28 - protein, 5 aa, Artificial Sequence
  Synthetic sequence
  MISC_FEATURE (1)..(5): Linker
GGGLS

SEQ ID NO: 29 - protein, 5 aa, Artificial Sequence
  Synthetic sequence
  MISC_FEATURE (1)..(5): Linker
GGGGL

SEQ ID NO: 30 - protein, 5 aa, Artificial Sequence
  Synthetic sequence
  MISC_FEATURE (1)..(5): Linker
LGGSG

SEQ ID NO: 31 - protein, 5 aa, Artificial Sequence
  Synthetic sequence
  MISC_FEATURE (1)..(5): Linker
GLGSG

SEQ ID NO: 32 - protein, 5 aa, Artificial Sequence
  Synthetic sequence
  MISC_FEATURE (1)..(5): Linker
GGLSG

SEQ ID NO: 33 - protein, 5 aa, Artificial Sequence
  Synthetic sequence
  MISC_FEATURE (1)..(5): Linker
GGGLG

SEQ ID NO: 34 - protein, 5 aa, Artificial Sequence
  Synthetic sequence
  MISC_FEATURE (1)..(5): Linker
GGGSL

SEQ ID NO: 35 - protein, 5 aa, Artificial Sequence
  Synthetic sequence
  MISC_FEATURE (1)..(5): Linker
LGGSS

SEQ ID NO: 36 - protein, 5 aa, Artificial Sequence
  Synthetic sequence
  MISC_FEATURE (1)..(5): Linker
GLGSS

SEQ ID NO: 37 - protein, 5 aa, Artificial Sequence
  Synthetic sequence
  MISC_FEATURE (1)..(5): Linker
GGLSS

SEQ ID NO: 38 - protein, 5 aa, Artificial Sequence
  Synthetic sequence
  MISC_FEATURE (1)..(5): Linker
LGLGS

SEQ ID NO: 39 - protein, 5 aa, Artificial Sequence
  Synthetic sequence
  MISC_FEATURE (1)..(5): Linker
GLGLS

SEQ ID NO: 40 - protein, 5 aa, Artificial Sequence
  Synthetic sequence
  MISC_FEATURE (1)..(5): Linker
GLLGS

SEQ ID NO: 41 - protein, 5 aa, Artificial Sequence
  Synthetic sequence
  MISC_FEATURE (1)..(5): Linker
LGGLS

SEQ ID NO: 42 - protein, 5 aa, Artificial Sequence
  Synthetic sequence
  MISC_FEATURE (1)..(5): Linker
GLGGL

SEQ ID NO: 43 - protein, 5 aa, Artificial Sequence
  Synthetic sequence
  MISC_FEATURE (1)..(5): Linker
LGLSG

SEQ ID NO: 44 - protein, 5 aa, Artificial Sequence
  Synthetic sequence
  MISC_FEATURE (1)..(5): Linker
GLLSG

SEQ ID NO: 45 - protein, 5 aa, Artificial Sequence
  Synthetic sequence
  MISC_FEATURE (1)..(5): Linker
GGLSL

SEQ ID NO: 46 - protein, 5 aa, Artificial Sequence
  Synthetic sequence
  MISC_FEATURE (1)..(5): Linker
GGLLG

SEQ ID NO: 47 - protein, 5 aa, Artificial Sequence
  Synthetic sequence
  MISC_FEATURE (1)..(5): Linker
GLGSL

SEQ ID NO: 48 - protein, 5 aa, Artificial Sequence
  Synthetic sequence
  MISC_FEATURE (1)..(5): Linker
LGLSS

SEQ ID NO: 49 - protein, 5 aa, Artificial Sequence
  Synthetic sequence
  MISC_FEATURE (1)..(5): Linker
GGLLS

SEQ ID NO: 50 - protein, 10 aa, Artificial Sequence
  Synthetic sequence
  MISC_FEATURE (1)..(5): Linker
LGGGSGGGGS

SEQ ID NO: 51 - protein, 10 aa, Artificial Sequence
  Synthetic sequence
  MISC_FEATURE (1)..(10): Linker
GLGGSGGGGS

SEQ ID NO: 52 - protein, 10 aa, Artificial Sequence
  Synthetic sequence
  MISC_FEATURE (1)..(10): Linker
GGLGSGGGGS

SEQ ID NO: 53 - protein, 10 aa, Artificial Sequence
  Synthetic sequence
  MISC_FEATURE (1)..(10): Linker
GGGLSGGGGS

SEQ ID NO: 54 - protein, 10 aa, Artificial Sequence
  Synthetic sequence
  MISC_FEATURE (1)..(10): Linker
GGGGLGGGGS

SEQ ID NO: 55 - protein, 10 aa, Artificial Sequence
  Synthetic sequence
  MISC_FEATURE (1)..(10): Linker
LGGSGGGGSG

SEQ ID NO: 56 - protein, 10 aa, Artificial Sequence
  Synthetic sequence
  MISC_FEATURE (1)..(10): Linker
GLGSGGGGSG

SEQ ID NO: 57 - protein, 10 aa, Artificial Sequence
  Synthetic sequence
  MISC_FEATURE (1)..(10): Linker
GGLSGGGGSG

SEQ ID NO: 58 - protein, 10 aa, Artificial Sequence
  Synthetic sequence
  MISC_FEATURE (1)..(10): Linker
GGGLGGGGSG

SEQ ID NO: 59 - protein, 10 aa, Artificial Sequence
  Synthetic sequence
  MISC_FEATURE (1)..(10): Linker
GGGSLGGGSG

SEQ ID NO: 60 - protein, 10 aa, Artificial Sequence
  Synthetic sequence
  MISC_FEATURE (1)..(10): Linker
LGGSSGGGSS

SEQ ID NO: 61 - protein, 10 aa, Artificial Sequence
  Synthetic sequence
  MISC_FEATURE (1)..(10): Linker
GLGSSGGGSS

SEQ ID NO: 62 - protein, 10 aa, Artificial Sequence
  Synthetic sequence
  MISC_FEATURE (1)..(10): Linker
GGLSSGGGSS

SEQ ID NO: 63 - protein, 10 aa, Artificial Sequence
  Synthetic sequence
  MISC_FEATURE (1)..(10): Linker
GGGLSGGGSS

SEQ ID NO: 64 - protein, 10 aa, Artificial Sequence
  Synthetic sequence
  MISC_FEATURE (1)..(10): Linker
GGGSLGGGSS

SEQ ID NO: 65 - protein, 10 aa, Artificial Sequence
  Synthetic sequence
  MISC_FEATURE (1)..(10): Linker
LGGGSLGGGS

SEQ ID NO: 66 - protein, 10 aa, Artificial Sequence
  Synthetic sequence
  MISC_FEATURE (1)..(10): Linker
GLGGSGLGGS

SEQ ID NO: 67 - protein, 10 aa, Artificial Sequence
  Synthetic sequence
  MISC_FEATURE (1)..(10): Linker
GGLGSGGLGS

SEQ ID NO: 68 - protein, 10 aa, Artificial Sequence
  Synthetic sequence
  MISC_FEATURE (1)..(10): Linker
GGGLSGGGLS

SEQ ID NO: 69 - protein, 10 aa, Artificial Sequence
  Synthetic sequence
  MISC_FEATURE (1)..(10): Linker
GGGGLGGGGL

SEQ ID NO: 70 - protein, 10 aa, Artificial Sequence
  Synthetic sequence
  MISC_FEATURE (1)..(10): Linker
LGGSGLGGSG

SEQ ID NO: 71 - protein, 10 aa, Artificial Sequence
  Synthetic sequence
  MISC_FEATURE (1)..(10): Linker
GLGSGGLGSG

SEQ ID NO: 72 - protein, 10 aa, Artificial Sequence
  Synthetic sequence
  MISC_FEATURE (1)..(10): Linker
GGLSGGGLSG

SEQ ID NO: 73 - protein, 10 aa, Artificial Sequence
  Synthetic sequence
  MISC_FEATURE (1)..(10): Linker
GGGLGGGGLG

SEQ ID NO: 74 - protein, 10 aa, Artificial Sequence
  Synthetic sequence
  MISC_FEATURE (1)..(10): Linker
GGGSLGGGSL

SEQ ID NO: 75 - protein, 10 aa, Artificial Sequence
  Synthetic sequence
  MISC_FEATURE (1)..(10): Linker
LGGSSLGGSS

SEQ ID NO: 76 - protein, 10 aa, Artificial Sequence
  Synthetic sequence
  MISC_FEATURE (1)..(10): Linker
GLGSSGLGSS

SEQ ID NO: 77 - protein, 10 aa, Artificial Sequence
  Synthetic sequence
  MISC_FEATURE (1)..(10): Linker
GGLSSGGLSS

SEQ ID NO: 78 - protein, 16 aa, Artificial Sequence
  Synthetic sequence
  MISC_FEATURE (1)..(10): Linker
TQKSLSLSPGKGLGGL

SEQ ID NO: 79 - protein, 13 aa, Artificial Sequence
  Synthetic sequence
  MISC_FEATURE (1)..(10): Linker
SLSLSPGKGLGGL

SEQ ID NO: 80 - protein, 36 aa, Artificial Sequence
  Synthetic sequence
  MISC_FEATURE (1)..(36): Linker
GGSAGGSGSGSSGGSSGASGTGTAGGTGSGSGTGSG

SEQ ID NO: 81 - protein, 36 aa, Artificial Sequence
  Synthetic sequence
  MISC_FEATURE (1)..(36): Linker
GGSGGGSEGGGSEGGGSEGGGSEGGGSEGGGSGGGS

SEQ ID NO: 82 - protein, 9 aa, Influenza A virus
CTELKLSDY

SEQ ID NO: 83 - protein, 9 aa, Human cytomegalovirus
NLVPMVATV

SEQ ID NO: 84 - protein, 9 aa, Mycobacterium tuberculosis
KLVANNTRL

SEQ ID NO: 85 - protein, 5 aa, Artificial Sequence
  Synthetic sequence
  MISC_FEATURE (1)..(5): Linker
GLSGL

SEQ ID NO: 86 - protein, 711 aa, Artificial Sequence
  Synthetic sequence
ATGCAGGTCTCCACTGCTGCCCTTGCCGTCCTCCTCTGCACCATGGCTCTCTGCAACCAGGTCCTCTCTGCACCACTTGCTGCTGACACGCCGACCGCCTGCTGCTTCAGCTACACCTCCCGACAGATTCCACAGAATTTCATAGCTGACTACTTTGAGACGAGCAGCCAGTGCTCCAAGCCCAGTGTCATCTTCCTAACCAAGAGAGGCCGGCAGGTCTGTGCTGACCCCAGTGAGGAGTGGGTCCAGAAATACGTCAGTGACCTGGAGCTGAGTGCCGAGCTCAAAACCCCACTTGGTGACACAACTCACACAGAGCCCAAATCTTGTGACACACCTCCCCCGTGCCCAAGGTGCCCAGGCGGTGGAAGCAGCGGAGGTGGAAGTGGAGGACAGCCCCGAGAACCACAGGTGTACACCCTGCCCCCATCCCGGGAGGAGATGACCAAGAACCAGGTCAGCCTGACCTGCCTGGTCAAAGGCTTCTACCCCAGCGACATCGCCGTGGAGTGGGAGAGCAGCGGGCAGCCGGAGAACAACTACAACACCACGCCTCCCATGCTGGACTCCGACGGCTCCTTCTTCCTCTACAGCAAGCTCACCGTGGACAAGAGCAGGTGGCAGCAGGGGAACATCTTCTCATGCTCCGTGATGCATGAGGCTCTGCACAACCGCTTCACGCAGAAGAGCCTCTCCCTGTCTCCGGGTAAA

SEQ ID NO: 87 - protein, 10 aa, Artificial Sequence
  Synthetic sequence
  MISC_FEATURE (1)..(10): Linker
GGGSSGGGSG

SEQ ID NO: 88 - protein, 60 aa, Artificial Sequence
  Synthetic sequence
AGQVRIWATYQTMLDKIREVPEGWLIFVAEREELYVRVRNGFRKVLLEARTALPRGTGNE

SEQ ID NO: 89 - protein, 39 aa, Artificial Sequence
  Synthetic sequence
KPLDGEYFTLQIRGPERFEMFRELNEALELKDAQAGKEP

SEQ ID NO: 90 - protein, 5 aa, Artificial Sequence
  Synthetic sequence
  MISC_FEATURE (1)..(5): Linker
GLGGL

SEQ ID NO: 91 - protein, 6 aa, Artificial Sequence
  Synthetic sequence
  MISC_FEATURE (1)..(6): Linker
GSGGGA

SEQ ID NO: 92 - protein, 12 aa, Artificial Sequence
  Synthetic sequence
  MISC_FEATURE (1)..(12): Linker
GSGGGAGSGGGA

SEQ ID NO: 93 - protein, 18 aa, Artificial Sequence
  Synthetic sequence
  MISC_FEATURE (1)..(18): Linker
GSGGGAGSGGGAGSGGGA

SEQ ID NO: 94 - protein, 24 aa, Artificial Sequence
  Synthetic sequence
  MISC_FEATURE (1)..(24): Linker
GSGGGAGSGGGAGSGGGAGSGGGA

SEQ ID NO: 95 - protein, 10 aa, Artificial Sequence
  Synthetic sequence
  MISC_FEATURE (1)..(10): Linker
GENLYFQSGG

SEQ ID NO: 96 - protein, 10 aa, Artificial Sequence
  Synthetic sequence
  MISC_FEATURE (1)..(10): Linker
SGGGSSGGGS

SEQ ID NO: 97 - protein, 10 aa, Artificial Sequence
  Synthetic sequence
  MISC_FEATURE (1)..(10): Linker
GGGGSGGGGS

SEQ ID NO: 98 - protein, 10 aa, Artificial Sequence
  Synthetic sequence
  MISC_FEATURE (1)..(10): Linker
SSGGGSSGGG

SEQ ID NO: 99 - protein, 10 aa, Artificial Sequence
  Synthetic sequence
  MISC_FEATURE (1)..(10): Linker
GGSGGGGSGG

SEQ ID NO: 100 - protein, 10 aa, Artificial Sequence
  Synthetic sequence
  MISC_FEATURE (1)..(10): Linker
GSGSGSGSGS

SEQ ID NO: 101 - protein, 6 aa, Artificial Sequence
  Synthetic sequence
  MISC_FEATURE (1)..(6): Linker
GGGSSS

SEQ ID NO: 102 - protein, 15 aa, Artificial Sequence
  Synthetic sequence
  MISC_FEATURE (1)..(15): Linker
GGGSSGGGSSGGGSS

SEQ ID NO: 103 - protein, 8 aa, Artificial Sequence
  Synthetic sequence
  MISC_FEATURE (1)..(8): Linker
GLGGLAAA

SEQ ID NO: 104 - protein, 12 aa, Artificial Sequence
  Synthetic sequence
  MISC_FEATURE (1)..(12): Linker
KPEPKPAPAPKP

SEQ ID NO: 105 - protein, 12 aa, Artificial Sequence
  Synthetic sequence
  MISC_FEATURE (1)..(12): Linker
AEAAAKEAAAKA

SEQ ID NO: 106 - protein, 5 aa, Artificial Sequence
  Synthetic sequence
  MISC_FEATURE (1)..(5): Linker
EAAAK

SEQ ID NO: 107 - protein, 15 aa, Artificial Sequence
  Synthetic sequence
  MISC_FEATURE (1)..(15): Linker
PSRLEEELRRRLTEP

SEQ ID NO: 108 - protein, 8 aa, Artificial Sequence
  Synthetic sequence
  MISC_FEATURE (1)..(8): Linker
SACYCELS

SEQ ID NO: 109 - protein, 5 aa, Artificial Sequence
  Synthetic sequence
  MISC_FEATURE (1)..(5): Linker
GGGGS

SEQ ID NO: 110 - protein, 5 aa, Artificial Sequence
  Synthetic sequence
  MISC_FEATURE (1)..(5): Linker
EAAAK

SEQ ID NO: 111 - protein, 5 aa, Artificial Sequence
  Synthetic sequence
  MISC_FEATURE (1)..(5): Linker; the sequence can be repeated m times, where m is an integer
EAAAK

SEQ ID NO: 112 - protein, 6 aa, Artificial Sequence
  Synthetic sequence
  MISC_FEATURE (1)..(6): Linker
  MISC_FEATURE (1)..(5): Repeated m times, where m is an integer greater than or equal to 1
EAAKGS

SEQ ID NO: 113 - protein, 17 aa, Artificial Sequence
  Synthetic sequence
  MISC_FEATURE (1)..(17): Linker
GPSRLEEELRRRLTEPG

SEQ ID NO: 114 - protein, 12 aa, Artificial Sequence
  Synthetic sequence
  MISC_FEATURE (1)..(12): Linker
HEYGAEALERAG

SEQ ID NO: 115 - DNA, 36 bp, Artificial Sequence
  Synthetic sequence
  misc_feature (1)..(36): encodes CLEC9A ligand
tggcccaggttccacagcagcgtgttccacacccac

SEQ ID NO: 116 - DNA, 324 bp, Artificial Sequence
  Synthetic sequence
  misc_feature (1)..(324): encodes CCL19
atggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctcctgaccagccctgggtggatcgcatcatccgaagactgaagaagtcttctgccaagaacaaaggcaacagcaccagaaggagccctgtgtct

SEQ ID NO: 117 - DNA, 333 bp, Artificial Sequence
  Synthetic sequence
  misc_feature (1)..(333): hinge region derived from IgM
gccgaactcccgcccaaggtgtccgtgttcgtccctccccgcgatgggttcttcggcaatccacgaaaatccaaactgatttgtcaggccaccggcttctccccccgacagatccaggtgagttggctacgagagggtaaacaggtggggagcggagtgaccactgaccaggtgcaggccgaggccaaggaaagcggacccacaacatacaaagtgacaagcactctgacgattaaggagtcagactggctcggccaatccatgtttacatgccgggttgatcacagagggttgaccttccaacagaacgcatccagtatgtgcgttccagat

SEQ ID NO: 118 - DNA, 81 bp, Artificial Sequence
  Synthetic sequence
  misc_feature (1)..(81): dimerization unit
gagctcaaaaccccacttggtgacacaactcacacagagcccaaatcttgtgacacacctcccccgtgcccaaggtgccca

SEQ ID NO: 119 - DNA, 321 bp, Artificial Sequence
  Synthetic sequence
  misc_feature (1)..(321): dimerization unit
ggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 120 - DNA, 432 bp, Artificial Sequence
  Synthetic sequence
  misc_feature (1)..(432): Dimerization unit
gagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 121 - DNA, 105 bp, Artificial Sequence
  Synthetic sequence
  misc_feature (1)..(105): encodes dHLX protein
ggagaactggaggaattacttaaacatctcaaggagttgctcaaaggccctaggaagggagaactggaggaactcctcaaacatctcaaggagttactaaaggga

SEQ ID NO: 122 - DNA, 210 bp, Artificial Sequence
  Synthetic sequence
  misc_feature (1)..(210): targeting unit
gcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgcc

SEQ ID NO: 123 - DNA, 69 bp, Artificial Sequence
  Synthetic sequence
  misc_feature (1)..(69): encodes signal peptide
atgcaggtctccactgctgcccttgccgtcctcctctgcaccatggctctctgcaaccaggtcctctct

SEQ ID NO: 124 - protein, 12 aa, Artificial Sequence
  Synthetic sequence
  MISC_FEATURE (1)..(12): Linker
ELKTPLGDTTHT

SEQ ID NO: 125 - protein, 223 aa, Artificial Sequence
  Synthetic sequence
  MISC_FEATURE (1)..(223): RBD sequence of spike protein of SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 126 - DNA, 180 bp, Artificial Sequence
  Synthetic sequence
  misc_feature (1)..(180): Collagen trimerization domain
gctgggcaggtgaggatctgggccacataccagaccatgctggacaagatccgggaggtgccggagggctggctcatctttgtggccgagagggaagagctctatgtacgcgttagaaatggcttccggaaggtgctgctggaggcccggacagccctcccgagaggcacgggcaatgag

SEQ ID NO: 127 - protein, 27 aa, Artificial Sequence
  Synthetic sequence
  MISC_FEATURE (1)..(27): Exemplary trimerization unit
GYIPEAPRDGQAYVRKDGEWVLLSTFL

SEQ ID NO: 128 - DNA, 117 bp, Artificial Sequence
  Synthetic sequence
  misc_feature (1)..(117): Exemplary tetramerization unit
aagcctctggacggagagtatttcactctccagatccggggccccgaaaggttcgaaatgttccgggagcttaacgaggccttggagctgaaagacgcacaggccggaaaggaaccg

SEQ ID NO: 129 - protein, 25 aa, Artificial Sequence
  Synthetic sequence
  MISC_FEATURE (1)..(25): T cell epitope - pep08
HFAIGLALYYPSARIVYTACSHAAV

SEQ ID NO: 130 - protein, 16 aa, Artificial Sequence
  Synthetic sequence
  MISC_FEATURE (1)..(16): T cell epitope - pep18
FVNEFYAYLRKHFSMM

SEQ ID NO: 131 - protein, 15 aa, Artificial Sequence
  Synthetic sequence
  MISC_FEATURE (1)..(15): T cell epitope - pep25
LVKPSFYVYSRVKNL

SEQ ID NO: 132 - protein, 19 aa, Artificial Sequence
  Synthetic sequence
  MISC_FEATURE (1)..(19): signal peptide
MNFGLRLIFLVLTLKGVQC

SEQ ID NO: 133 - protein, 23 aa, Artificial Sequence
  Synthetic sequence
  MISC_FEATURE (1)..(23): Signal peptide
MQVSTAALAVLLCTMALCNQVLS

SEQ ID NO: 134 - protein, 6 aa, Artificial Sequence
  Synthetic sequence
  MISC_FEATURE (1)..(6): Linker
SGSSGS

SEQ ID NO: 135 - protein, 10 aa, Artificial Sequence
  Synthetic sequence
  MISC_FEATURE (1)..(10): Linker
GGGGSGGGGS

SEQ ID NO: 136 - protein, 5 aa, Artificial Sequence
  Synthetic sequence
  MISC_FEATURE (1)..(5): Linker; the motif can be repeated m times, where m is an integer
GGSGG

SEQ ID NO: 137 - protein, 6 aa, Artificial Sequence
  Synthetic sequence
  MISC_FEATURE (1)..(6): Linker; the motif can be repeated m times, where m is an integer
SGSSGS

SEQ ID NO: 138 - protein, 5 aa, Artificial Sequence
  Synthetic sequence
  MISC_FEATURE (1)..(5): Linker
GGSGG

SEQ ID NO: 139 - protein, 5 aa, Artificial Sequence
  Synthetic sequence
  MISC_FEATURE (1)..(5): Linker
GPGPG

SEQ ID NO: 140 - protein, 524 aa, Artificial Sequence
  Synthetic sequence
  MISC_FEATURE (1)..(524): Hemagglutinin of influenza H1N1
DTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLV

SEQ ID NO: 141 - DNA, 780 bp, Artificial Sequence
  Synthetic sequence
  misc_feature (1)..(780): DNA sequence encoding exemplary construct of SEQ ID NO 2
atgaacttcggactgagactcatttttctcgttctcactctgaagggcgtccagtgtgatattcagatgactcagacaaccagtagtctgtctgctagtctgggggatcgagttacaatctcttgttcagcatctcaggacattaataactatctgaactggtaccagcagaaacctgatggtaccgtcaagctcctgatctactatacatcttccctgcattccggtgtgccttctaggttcagtggctcaggatctggcactgactacagcctgaccatctctaacctggagccagaggacatcgcaacctactactgccagcagtacagcaaattcccacggaccttcggtgggggcacaaagctcgagattaagaggggcggcggagggagcggaggcggagggtcaggcggtggagggtctcagatccagctggtgcagagcggcccagaactgaaaaagccaggggaaacagtgaaaatctcctgcaaagcaagtggttacaccttcatcaattacgggatgaattgggttaagcagactccaggaaaaggcctgaagtggatgggctggatcaatacatattccggcgagccaacttaccctgacgactttaaaggaaggtttgcctttagtctcgaaacaagcgcctctacagcctacctgcagctcaacaatctcaagaacgaggacatggcaacttatttctgtgctagaggcgattactatgggccgtttgataattggggacagggtaccacactcaccgtttcctcc

SEQ ID NO: 142 - protein, 15 aa, Artificial Sequence
  Synthetic sequence
  MISC_FEATURE (1)..(15): Pep 01
RSFIEDLLFNKVTLA

SEQ ID NO: 143 - protein, 25 aa, Artificial Sequence
  Synthetic sequence
  MISC_FEATURE (1)..(25): Pep 02
MTYRRLISMMGFKMNYQVNGYPNMF

SEQ ID NO: 144 - protein, 15 aa, Artificial Sequence
  Synthetic sequence
  MISC_FEATURE (1)..(15): Pep 03
LMIERFVSLAIDAYP

SEQ ID NO: 145 - protein, 15 aa, Artificial Sequence
  Synthetic sequence
  MISC_FEATURE (1)..(15): Pep 04
RAMPNMLRIMASLVL

SEQ ID NO: 146 - protein, 15 aa, Artificial Sequence
  Synthetic sequence
  MISC_FEATURE (1)..(15): Pep 05
MVYMPASWVMRIMTW

SEQ ID NO: 147 - protein, 17 aa, Artificial Sequence
  Synthetic sequence
  MISC_FEATURE (1)..(17): Pep 06
FLNRFTTTLNDFNLVAM

SEQ ID NO: 148 - protein, 18 aa, Artificial Sequence
  Synthetic sequence
  MISC_FEATURE (1)..(18): Pep 07
SSVELKHFFFAQDGNAAI

SEQ ID NO: 149 - protein, 15 aa, Artificial Sequence
  Synthetic sequence
  MISC_FEATURE (1)..(15): Pep 09
YFIKGLNNLNRGMVL

SEQ ID NO: 150 - protein, 15 aa, Artificial Sequence
  Synthetic sequence
  MISC_FEATURE (1)..(15): Pep 10
YLNTLTLAVPYNMRV

SEQ ID NO: 151 - protein, 16 aa, Artificial Sequence
  Synthetic sequence
  MISC_FEATURE (1)..(16): Pep 11
AQFAPSASAFFGMSRI

SEQ ID NO: 152 - protein, 15 aa, Artificial Sequence
  Synthetic sequence
  MISC_FEATURE (1)..(15): Pep 12
EIVDTVSALVYDNKL

SEQ ID NO: 153 - protein, 28 aa, Artificial Sequence
  Synthetic sequence
  MISC_FEATURE (1)..(28): Pep 13
SSGDATTAYANSVFNICQAVTANVNALL

SEQ ID NO: 154 - protein, 17 aa, Artificial Sequence
  Synthetic sequence
  MISC_FEATURE (1)..(17): Pep 14
HVISTSHKLVLSVNPYV

SEQ ID NO: 155 - protein, 24 aa, Artificial Sequence
  Synthetic sequence
  MISC_FEATURE (1)..(24): Pep 15
MLSDTLKNLSDRVVFVLWAHGFEL

SEQ ID NO: 156 - protein, 18 aa, Artificial Sequence
  Synthetic sequence
  MISC_FEATURE (1)..(18): Pep 17
ASIKNFKSVLYYQNNVFM

SEQ ID NO: 157 - protein, 15 aa, Artificial Sequence
  Synthetic sequence
  MISC_FEATURE (1)..(15): Pep 19
RVWTLMNVLTLVYKV

SEQ ID NO: 158 - protein, 15 aa, Artificial Sequence
  Synthetic sequence
  MISC_FEATURE (1)..(15): Pep 20
FAYANRNRFLYIIKL

SEQ ID NO: 159 - protein, 27 aa, Artificial Sequence
  Synthetic sequence
  MISC_FEATURE (1)..(27): Pep 01 H1N1
KLSTRGVQIASNENMETMESSTLELRS

SEQ ID NO: 160 - protein, 27 aa, Artificial Sequence
  Synthetic sequence
  MISC_FEATURE (1)..(27): pep 02
GENGRKTRIAYERMCNILKGKFQTAAQ

SEQ ID NO: 161 - protein, 27 aa, Artificial Sequence
  Synthetic sequence
  MISC_FEATURE (1)..(27): pep 03
GKMIGGIGRFYIQMCTELKLSDYEGRL

SEQ ID NO: 162 - protein, 27 aa, Artificial Sequence
  Synthetic sequence
  MISC_FEATURE (1)..(27): pep 04
LLEMCHSTQIGGIRMVDILRQNPTEEQ

SEQ ID NO: 163 - protein, 27 aa, Artificial Sequence
  Synthetic sequence
  MISC_FEATURE (1)..(27): pep 05
IWHSNLNDATYQRTRALVRTGMDPRMC

SEQ ID NO: 164 - protein, 27 aa, Artificial Sequence
  Synthetic sequence
  MISC_FEATURE (1)..(27): Pep 06
DQSLPPNFSSLENFRAYVDGFEPNGYI

SEQ ID NO: 165 - protein, 27 aa, Artificial Sequence
  Synthetic sequence
  MISC_FEATURE (1)..(27): Pep 07
MSLLTEVETPIRNEWGCRCNGSSDPLA

SEQ ID NO: 166 - protein, 27 aa, Artificial Sequence
  Synthetic sequence
  MISC_FEATURE (1)..(27): Pep 08
YEGRLIQNSLTIERMVLSAFDERRNKY

SEQ ID NO: 167 - protein, 27 aa, Artificial Sequence
  Synthetic sequence
  MISC_FEATURE (1)..(27): Pep 09
NSSLCPIRGWAIYSKDNSIRIGSKGDV

SEQ ID NO: 168 - protein, 27 aa, Artificial Sequence
  Synthetic sequence
  MISC_FEATURE (1)..(27): pep 10
RNFWRGENGRKTRIAYERMCNILKGKF

SEQ ID NO: 169 - protein, 577 aa, Artificial Sequence; identical to SEQ ID NO: 17
  Synthetic sequence
  MISC_FEATURE (1)..(577): TECH004-IV002

SEQ ID NO: 170 - protein, 639 aa, Artificial Sequence
  Synthetic sequence
  MISC_FEATURE (1)..(639): TECH004-IV003
MQVSTAALAVLLCTMALCNQVLSAPLAADTPTACFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSYFIKGLNNLNRGMVLGGGGSGGGGSSSGDATTAYANSVFNICQAVTANVNALLGGGGSGGGGSLVKPSFYVYSRVKNLGGSAGGSGSGSSGGSSGASGTGTAGGTGSGSGTGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 171 - protein, 770 aa, Artificial Sequence
  Synthetic sequence
  MISC_FEATURE (1)..(770): TECH004-IV004
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMTYRRLISMMGFKMNYQVNGYPNMFGGGGSGGGGSYFIKGLNNLNRGMVLGGGGSGGGGSHFAIGLALYYPSARIVYTACSHAAVGGGGSGGGGSMVYMPASWVMRIMTWGGGGSGGGGSFAYANRNRFLYIIKLSGGGGSGGGGFLNRFTTTLNDFNLVAMSSVELKHFFFAQDGNAAISGGGGSGGGGSSGDATTAYANSVFNICQAVTANVNALLSGGGGSGGGGFVNEFYAYLRKHFSMMGGGGSGGGGSLVKPSFYVYSRVKNLGGSAGGSGSGSSGGSSGASGTGTAGGTGSGSGTGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 172 - protein, 975 aa, Artificial Sequence
  Synthetic sequence
  MISC_FEATURE (1)..(975): TECH004-IV005
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMTYRRLISMMGFKMNYQVNGYPNMFGGGGSGGGGSHVISTSHKLVLSVNPYVASIKNFKSVLYYQNNVFMGGGGSGGGGSYFIKGLNNLNRGMVLYLNTLTLAVPYNMRVGGGGSGGGGSMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSRAMPNMLRIMASLVLMVYMPASWVMRIMTWGGGGSGGGGSRVWTLMNVLTLVYKVFAYANRNRFLYIIKLSGGGGSGGGGAQFAPSASAFFGMSRIEIVDTVSALVYDNKLSGGGGSGGGGFLNRFTTTLNDFNLVAMSSVELKHFFFAQDGNAAISGGGGSGGGGSSGDATTAYANSVFNICQAVTANVNALLSGGGGSGGGGFVNEFYAYLRKHFSMMGGGGSGGGGSLVKPSFYVYSRVKNLGGSAGGSGSGSSGGSSGASGTGTAGGTGSGSGTGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 173 - protein, 903 aa, Artificial Sequence
  Synthetic sequence
  MISC_FEATURE (1)..(903): TECH004-IV025
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKLSTRGVQIASNENMETMESSTLELRSGGGGSGGGGSDQSLPPNFSSLENFRAYVDGFEPNGYIGGGGSGGGGSMSLLTEVETPIRNEWGCRCNGSSDPLAGGSAGGSGSGSSGGSSGASGTGTAGGTGSGSGTGSGDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLV

SEQ ID NO: 174 - protein, 977 aa, Artificial Sequence
  Synthetic sequence
  MISC_FEATURE (1)..(977): TECH004-IV026
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKLSTRGVQIASNENMETMESSTLELRSGGGGSGGGGSGENGRKTRIAYERMCNILKGKFQTAAQGGGGSGGGGSIWHSNLNDATYQRTRALVRTGMDPRMCGGGGSGGGGSDQSLPPNFSSLENFRAYVDGFEPNGYIGGGGSGGGGSMSLLTEVETPIRNEWGCRCNGSSDPLAGGSAGGSGSGSSGGSSGASGTGTAGGTGSGSGTGSGDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLV

SEQ ID NO: 175 - protein, 1162 aa, Artificial Sequence
  Synthetic sequence
  MISC_FEATURE (1)..(1162): TECH004-IV027
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKLSTRGVQIASNENMETMESSTLELRSGGGGSGGGGSGENGRKTRIAYERMCNILKGKFQTAAQGGGGSGGGGSGKMIGGIGRFYIQMCTELKLSDYEGRLGGGGSGGGGSLLEMCHSTQIGGIRMVDILRQNPTEEQGGGGSGGGGSIWHSNLNDATYQRTRALVRTGMDPRMCGGGGSGGGGSDQSLPPNFSSLENFRAYVDGFEPNGYIGGGGSGGGGSMSLLTEVETPIRNEWGCRCNGSSDPLAGGGGSGGGGSYEGRLIQNSLTIERMVLSAFDERRNKYGGGGSGGGGSNSSLCPIRGWAIYSKDNSIRIGSKGDVGGGGSGGGGSRNFWRGENGRKTRIAYERMCNILKGKFGGSAGGSGSGSSGGSSGASGTGTAGGTGSGSGTGSGDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLV

SEQ ID NO: 176 - protein, 13 aa, Artificial Sequence
  Synthetic sequence
  MISC_FEATURE (1)..(13): Pep11
SVSSFERFEIFPK

SEQ ID NO: 177 - protein, 14 aa, Artificial Sequence
  Synthetic sequence
  MISC_FEATURE (1)..(14): Pep12
HNTNGVTAACSHEG

SEQ ID NO: 178 - protein, 9 aa, Artificial Sequence
  Synthetic sequence
  MISC_FEATURE (1)..(9): Pep13
IYSTVASSL

SEQ ID NO: 179 - protein, 565 aa, Artificial Sequence
  Synthetic sequence
  MISC_FEATURE (1)..(565): Hemagglutinin/HA Protein
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

(19) United States
(12) Patent Application Publication
(10) Pub. No.: US 2025/0339509 A1
(43) Pub. Date: Nov. 6, 2025

(54) IMMUNOGENIC CONSTRUCTS AND VACCINES FOR USE IN THE PROPHYLACTIC AND THERAPEUTIC TREATMENT OF INFECTIOUS DISEASES

(71) Applicant: Nykode Therapeutics ASA, Oslo (NO)

(72) Inventors: Agnete Brunsvik Fredriksen, Rælingen (NO); Gunnstein Norheim, Oslo (NO); Elisabeth Stubsrud, Oslo (NO)

(73) Assignee: Nykode Therapeutics ASA, Oslo (NO)

(21) Appl. No.: 18/558,492

(22) PCT Filed: May 3, 2022
(86) PCT No.: PCT/EP2022/061819

§ 371 Date: Nov. 1, 2023

(30) Foreign Application Priority Data
May 3, 2021 (DK) PA 2021 70205

(51) Int. Cl. A61K 39/215; A61K 38/00; A61K 39/00; A61P 31/14; C07K 14/005; C07K 14/47; C07K 14/78; C12N 7/00

(52) U.S. Cl. CPC A61K 39/215; A61P 31/14; C07K 14/005; C07K 14/4746; C07K 14/78; C12N 7/00; A61K 38/00; A61K 2039/572; C07K 2319/02; C07K 2319/30; C12N 2770/20022; C12N 2770/20034

(57) ABSTRACT

This invention relates to immunogenic constructs, such as polynucleotides, polypeptides and dimeric proteins, and vaccines comprising such immunogenic constructs, which are useful for the prophylactic and therapeutic treatment of infectious diseases, as well as methods for producing and using the immunogenic constructs and vaccines.

### CROSS-REFERENCE TO RELATED APPLICATIONS

This application is a National Stage Entry of PCT/EP2022/061819, filed May 3, 2022, which claims the benefit of priority to Denmark Patent Application No. PA 2021 70205, filed May 3, 2021, which are hereby incorporated by reference in their entirety.

### TECHNICAL FIELD

This invention relates to immunogenic constructs, such as polynucleotides, polypeptides and multimeric proteins, such as dimeric proteins, and vaccines comprising such immunogenic constructs, which are useful for the prophylactic and therapeutic treatment of infectious diseases, as well as methods for producing and using the immunogenic constructs and vaccines.

### REFERENCE TO AN ELECTRONIC SEQUENCE LISTING

The instant application contains a Sequence Listing which has been submitted in ASCII (ST.25) format via USPTO Patent Center, which is hereby incorporated by reference into the specification in its entirety. Said ASCII copy, named “V89540_1120US_SL”, was created on Jun. 11, 2024, and is 192,708 bytes in size.

### BACKGROUND

Both B cell (humoral/antibody mediated) and immune system cell responses are important components of protective responses against infections caused by pathogens. Specific antibodies against pathogen antigens can mediate a broad range of effector functions, such as e.g. a) direct neutralization of toxins or pathogens, b) neutralization of pathogen virulence factors, c) binding to and trapping of pathogens in mucins, d) activating complement to mediate anti-pathogen phagocytic clearance, degradation or lysis, e) activating neutrophil opsonophagocytosis, f) inducing macrophage opsonophagocytosis g) activating natural killer (NK) cell degranulation to kill infected cells, h) enhancing antigen update, processing and presentation by dendritic cells to T and B cells, i) inducing degranulation of mast cells, basophils and eosinophils in the setting of parasitic infections (L. Lu et al., Nat Rev Immunol 18(1), 2018, 46).

Complementing these activities, T cell responses are critical for limiting viral replication and infection by killing the infected cells, inducing apoptosis, releasing antiviral substances, and/or inducing increased intracellular lysis in already infected cells and thus help to prevent, reduce severity of or cure the disease. In addition, effective and long-lasting response in both arms of immunity usually requires additional support from T-helper (Th1 and Th2) lymphocytes.

Cytotoxic T lymphocytes (CTL) also play a significant role (F. Sheperd et al., Int J Mol Sci 21, 2020, 6144) with e.g. intracellular pathogens where MHC class I-restricted CD8+ T cells are critical for clearing bacterial infections and are known to provide protective immunity against a range of bacterial species. MHC class II restricted CD4+ T cells support memory CD8+ T cell responses and are important for protective immunity against bacterial infections. Naïve CD4+ T cells differentiate subsets of cells with effector capacity, such as T helper 1 (Th1) and Th2 cells. After binding specific T cell epitopes on the surface of antigen-presenting cells (APCs), Th1 and Th2 cells supply specific soluble cytokine signals that regulate the balance between antibody and CTL immunity. Thus, effective immunity involves multiple antigen recognition events of specific pathogen immunogenic determinants (epitopes) by T-helper cells followed by molecular recognition by B cells, CTL, or both.

Different types of lymphocytes (B cells, CTLs and Th cells) specifically recognize different types of epitopes of the pathogen. B cell epitopes can be categorized as linear or conformational epitopes, with linear epitopes often being parts of conformational B-cell epitopes in native proteins. Conformational epitopes are exposed structural features on the surface of pathogens such as a viral envelope, bacterial outer membrane or secreted bacterial toxins. T cell epitopes are short peptides from any protein of a pathogen, which only have to conform to the host antigen-processing and MHC binding mechanisms, most notably class I or class II MHC haplotype restriction mechanisms. Suitable T cell epitopes occur with an estimated frequency of about one per 200-500 amino acid sequence, depending on host population and pathogen. Therefore, it is likely that a naturally occurring protein antigen does not comprise or only comprises few suitable T cell epitopes, or has only suboptimal T cell epitopes.

Combining in a vaccine one or more selected T cell epitopes and a B cell antigen is beneficial: while the presence of the antigen ensures the production of persistent and functional antibodies, the presence of T cell epitopes will elicit strong T cell response with a long-lasting memory population; in totality providing protection against subsequent infection.

By including T cell epitopes which target pathogen proteins that are not surface proteins and thus mutate less frequently, a vaccine may provide sufficient protection against infection with a pathogen, even if the included B cell antigen is no longer optimal. If the included T cell epitopes are conserved T cell epitopes, e.g. between subgenus, species or strains, there is an even greater likelihood that the vaccine renders protection against future mutated pathogens and future similar pathogens. Combination of multiple antigen serotypes or T cell epitopes from divergent clades may be required to provide a broadly protective immune response across populations.

The vaccibody construct is a dimeric protein consisting of two polypeptides, each comprising:
a) a targeting unit, which targets antigen-presenting cells,
b) a dimerization unit, and
c) an antigenic unit and which, after administration to a subject, has shown to be efficient in generating an immune response against the antigens or epitopes comprised in the antigenic unit.
   In some embodiments, the vaccibody construct is a multimeric protein consisting of multiple polypeptides, each comprising a targeting unit, which targets antigen-presenting cells, a multimerization unit and an antigenic unit and which, after administration to a subject, has shown to be efficient in generating an immune response against the antigens or epitopes comprised in the antigenic unit.

The vaccibody construct may be administered to a subject in the form of a polynucleotide (e.g. a DNA plasmid) comprising a nucleotide sequence encoding the polypeptide. After administration to host cells, e.g. muscle cells of a human, the polypeptide is expressed which, due to the multimerization unit, forms a dimeric protein; e.g. the polypeptide is expressed which, due to the dimerization unit, forms a dimeric protein.

### SUMMARY

The present disclosure provides a vaccibody construct or a polynucleotide encoding same, which comprises an antigenic unit that comprises one or more T cell epitopes and one or more antigens which are arranged in a specific way.

Thus, in a first aspect, the disclosure provides an immunogenic construct comprising:
(i) a polynucleotide comprising a nucleotide sequence encoding a polypeptide comprising:
   a) a targeting unit targeting or capable of targeting antigen-presenting cells,
   b) a multimerization unit such as a dimerization unit, and
   c) an antigenic unit comprising one or more T cell epitopes and one or more antigens or parts or fragments thereof, or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
wherein the antigenic unit comprises a subunit comprising the T cell epitopes which are separated from each other by T cell epitope linkers, if more than one T cell epitope is comprised in the subunit; and wherein the subunit is connected to the multimerization unit by a unit linker and separated from the one or more antigens or parts or fragments thereof by an antigen linker.

Preferably, the T cell epitopes and the antigens or parts or fragments thereof are from a pathogen.

Such a construct will, once administered to a human in the form of a vaccine, elicit a rapid, strong and persistent T cell response and B cell response and is thus suitable as a prophylactic or therapeutic vaccine for an infectious disease.

This is of particular importance in a pandemic or epidemic, where time is of essence to stop a pathogen from spreading further. For diseases where infected individuals may be asymptotic or only show mild and/or diffuse symptoms, there may not be time to test whether an individual is already infected or not. Moreover, tests may not be available, not be available at sufficient numbers or not be specific enough. The time from exposure to infection, severe disease and/or ultimately death may vary according to pathogen species, and for some pathogen species this time window will allow a therapeutic use of the vaccine of same design as for preventive purpose (i.e. ranging from post-exposure prophylaxis to early stage treatment). Thus, being able to provide prophylactic or therapeutic treatment in a single vaccine is a huge benefit.

The immunogenic construct of the disclosure may be used in a vaccine, i.e. a composition comprising the construct of the disclosure and a pharmaceutically acceptable carrier, for use in the prophylactic or therapeutic treatment of an infectious disease, by administering the vaccine to a subject.

### DESCRIPTION OF THE DRAWINGS

FIG. 1 shows an example of an immunogenic construct of the disclosure described as a polypeptide having an N-terminal start and a C-terminal end. The elements of the polypeptide—targeting unit (TU), dimerization unit (DimU) and antigenic unit—may be arranged in the polypeptide such that the antigenic unit is located at the C-terminal end of the polypeptide (FIG. 1a) or at the N-terminal start of the polypeptide (FIG. 1b). Further details are provided in the section with the title “Immunogenic construct”.

FIG. 2 shows a schematic drawing of the immunogenic constructs VB2081-VB2099 of Example 2, shown as dimeric protein.

FIG. 3: The proteins encoded in the immunogenic constructs VB2081-VB2099 were expressed and secreted 6 days after transfection of HEK293 cells. Conformational integrity of the proteins was confirmed by binding to antibodies detecting human IgG CH3 domain (comprised in the dimerization unit) and the RBD (Wuhan variant) antigen (antigenic unit) in ELISA.

FIG. 4: Anti-RBD (Wuhan variant) IgG immune response in mice vaccinated with 1 dose of 25 μg of the indicated immunogenic constructs. Mice were vaccinated by intramuscular administration of DNA on day 0, immediately followed by electroporation of the injection site. Sera obtained at day 14 post vaccination were tested for anti-RBD IgG antibodies binding the Wuhan RBD protein. Mean of 2-5 mice per group is shown. Anti-RBD IgG immune response was compared to that induced by VB2060 (comprising the same antigen but lacking the subunit with T cell epitopes) in mice which were treated identically.

FIG. 5: T cell response induced by immunogenic constructs VB2099, VB2097, VB2095, VB2089, VB2084, VB2087, VB2082 and VB2081. Mice (5 animals/group) were vaccinated intramuscularly on day 0 with 25 μg of the indicated immunogenic construct (DNA plasmids). On day 14 post vaccination, the spleens were harvested and the splenocytes were re-stimulated with the predicted T cell epitope(s) and the 15-mer overlapping peptides covering the Wuhan RBD aa sequence. The figure represents the total number of IFN-γ positive spots/1×10⁶ splenocytes.

FIG. 6: Expression and secretion levels of proteins/polypeptides encoded by DNA plasmids TECH004-IV002, TECH004-IV003, TECH004-IV004 and TECH004-IV005. Proteins were detected in the supernatant of Expi293F cells transfected with said DNA plasmids by the enzyme-linked immunosorbent assay (ELISA) using mouse a-human IgG CH3 domain capture Ab (MCA878G), rabbit a-SARS-CoV-2/2019-nCoV RBD detection Ab (40592-T62), and goat anti-rabbit IgG-HRP secondary Ab (31460). “Expifect”: cells treated only with transfection agent Expifectamine which serve as a negative control.

FIG. 7: Expression and secretion of intact proteins encoded by some constructs of the disclosure. Western blot shows full-length secretion of the first polypeptide. Reduced supernatant samples from transfection control, TECH004-IV002, TECH004-IV003 TECH004-IV004, and TECH004-IV005. Primary antibody: goat anti-human MIP-1α (AF270). Secondary antibody: donkey anti-goat, Dylight 800 (SA5-10092). Chemidoc channels Dylight 800 and 650 (for protein standard).

FIG. 8: Immunogenicity of DNA plasmids TECH004-IV002, TECH004-IV003, TECH004-IV004, and TECH004-IV005 against the encoded T cell epitopes in mice vaccinated with these plasmids by measuring the IFN-γ secretion from T cells (total T cell response), compared to the negative control VB1026.

FIG. 9: Immunogenicity of DNA plasmids TECH004-IV002, TECH004-IV003, TECH004-IV004, and TECH004-IV005 against Wuhan RBD Antigenic unit in mice vaccinated with these plasmids by measuring the IFN-γ secretion from T cells (total T cell response), compared to the negative control VB1026.

FIG. 10: immunogenicity of DNA plasmids TECH004-IV002, TECH004-IV003, TECH004-IV004, and TECH004-IV005 in mice vaccinated with said plasmids by measuring total IgG antibodies binding the Wuhan RBD protein, compared to the negative control VB1026.

FIG. 11: Expression and secretion levels of proteins encoded by constructs of the disclosure. Protein expression and secretion levels of the polypeptides encoded by DNA plasmids TECH004-IV025, TECH004-IV026 and TECH004-IV027 were detected in the supernatant of Expi293F cells transfected with said DNA plasmids by the enzyme-linked immunosorbent assay (ELISA) using mouse anti-human IgG CH3 domain capture Ab (MCA878G), rabbit anti-Influenza A H1N1 HA domain detection Ab (11684-R107), and goat anti-rabbit IgG secondary Ab (31460). “Expifect”: cells treated only with transfection agent Expifectamine which serve as a negative control.

FIG. 12: Immunogenicity of DNA plasmids TECH004-IV025, TECH004-IV026, and TECH004-IV027 against the encoded T cell epitopes in mice vaccinated with these plasmids by measuring the IFN-γ secretion from T cells (total T cell response), compared to the negative control VB1026.

FIG. 13: Immunogenicity of DNA plasmids TECH004-IV025, TECH004-IV026, and TECH004-IV027 against H1N1 HA Antigenic unit in mice vaccinated with these plasmids by measuring the IFN-γ secretion from T cells (total T cell response), compared to the negative control VB1026.

FIG. 14: Immunogenicity of DNA plasmids TECH004-IV025, TECH004-IV026, and TECH004-IV027 in mice vaccinated with said plasmids by way of measuring total IgG antibodies binding the H1N1 HA protein, compared to the negative control VB1026.

### DETAILED DESCRIPTION

An “immunogenic construct” is one that elicits an immune response, particularly when administered to a subject in a form suitable for administration and in an amount effective to elicit the immune response (i.e. an immunologically effective amount).

A “subject” is an animal, e.g. a mouse, or a human. A subject may be a patient, i.e. a human suffering from an infectious disease who is in need of a therapeutic treatment, or it may be a subject in need of prevention from being infected with an infectious disease, or it may be a subject suspected of suffering from an infectious disease. The terms “subject” and “individual” are used interchangeably herein.

An “infectious disease” is a disease caused by one or more pathogens, including viruses, bacteria, fungi and parasites.

A “treatment” is a prophylactic treatment or a therapeutic treatment.

A “prophylactic treatment” is a treatment administered to a subject who does not (or not yet) display signs or symptoms of, or displays only early signs or symptoms of, an infectious disease, such that treatment is administered for the purpose of preventing or decreasing the risk of developing the disease and/or symptoms associated with the disease. A prophylactic treatment functions as a preventive treatment against an infectious disease, or as a treatment that inhibits or reduces further development or enhancement of the disease and/or its associated symptoms. The terms prophylactic treatment, prophylaxis and prevention are used interchangeably herein.

A “therapeutic treatment” is a treatment administered to a subject who displays symptoms or signs of an infectious disease, in which treatment is administered to the subject for the purpose of diminishing or eliminating the disease and/or those signs or symptoms.

A “T cell epitope” as used herein refers to a single T cell epitope or a part or region of an antigen containing multiple T cell epitopes, e.g. multiple minimal epitopes.

A “part” of an antigen is a fragment or portion of an antigen, i.e. part/fragment of the amino acid sequence of an antigen, or the nucleotide sequence encoding same, e.g. an epitope; preferably, the part or fragment of the antigen is immunogenic. These terms will be used throughout interchangeably.

The term “minimal epitope” refers to a subsequence of an epitope predicted to bind to MHC I or MHC II. In other words, the minimal epitope may be immunogenic, i.e. capable of eliciting an immune response. The term minimal epitope thus may refer to short subsequences of an epitope, which are predicted to bind to MHC I or MHC II. A 27-mer epitope may thus encompass several minimal epitopes, which may each have a length shorter than 27 amino acids, and which each are immunogenic. For example, a minimal epitope could consist of the first 14 amino acids of the epitope, provided that it is predicted to bind to MHC I or MHC II, or it could consist of amino acids 9 to 18 of the epitope, or of amino acids 7 to 22, provided that these sequences are predicted to bind to MHC I or MHC II.

A “nucleotide sequence” is a sequence consisting of nucleotides. The terms “nucleotide sequence” and “nucleic acid sequence” are used interchangeably herein.

The section headings used herein are for organizational purposes only and are not to be construed as limiting the subject matter described.

### Immunogenic Construct

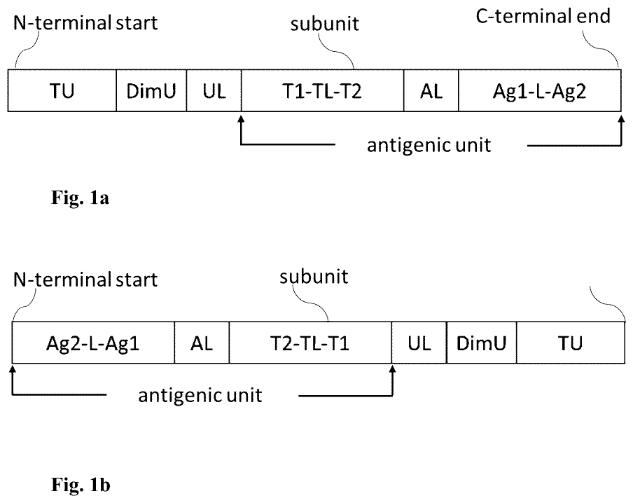

The immunogenic construct of the disclosure can be described as a polypeptide having an N-terminal start and a C-terminal end (illustrated in FIG. 1, which shows an embodiment where the construct comprises a multimerization unit which is a dimerization unit). The elements and units of the polypeptide—targeting unit (TU), multimerization unit, such as, in this Figure, a dimerization unit (DimU), and antigenic unit (Ag)—may be arranged in the polypeptide such that the antigenic unit is located at the C-terminal end of the polypeptide (FIG. 1a) or at the N-terminal start of the polypeptide (FIG. 1b). Preferably, the antigenic unit is located at the C-terminal end of the polypeptide.

The elements of the antigenic unit—the subunit with the T cell epitope(s) and T cell epitope linker(s) comprised in the subunit, if the subunit comprises more than one T cell epitope, the antigen linker (AL) and the one or more antigens or parts or fragments thereof—are arranged such that the subunit is connected to the multimerization unit, such as a dimerization unit, by a unit linker (UL) and separated from the one or more antigens or parts (or fragments) thereof by an antigen linker (AL). Thus, the subunit with the T cell epitope(s) is closest to the multimerization unit, such as a dimerization unit, while the antigen(s) constitute the terminal end of the polypeptide.

FIG. 1 illustrates an antigenic unit with 2 T cell epitopes (T1, T2) and 2 antigens or parts of 2 antigens or 2 parts of one antigen (Ag1, Ag2). The 2 T cell epitopes in the subunit are separated by a T cell epitope linker (TL). The subunit is separated from the first antigen or part thereof (Ag1) by the antigen linker (AL). The 2 antigens or parts of 2 antigens or 2 parts of one antigen are separated by a linker (L).

Also, the antigenic unit may be described as a polypeptide having an N-terminal start (which, in FIG. 1a, is the start of the subunit and which, in FIG. 1b, is the start of the antigenic unit) and a C-terminal end (in FIG. 1a, the end of the antigenic unit and in FIG. 1b, the end of the subunit). The elements of the antigenic unit may be arranged in said polypeptide such that the one or more antigens or parts thereof are located at the C-terminal end of the antigenic unit (FIG. 1a) or at the N-terminal start of the antigenic unit (FIG. 1b). Preferably, the one or more antigens or parts thereof are located at the C-terminal end of the antigenic unit.

Within the subunit comprising more than one T cell epitopes, the T cell epitopes are separated by T cell epitope linkers (TL in FIG. 1). A subunit that comprises n T cell epitopes, where n is an integer, preferably comprises n−1 T cell epitope linkers.

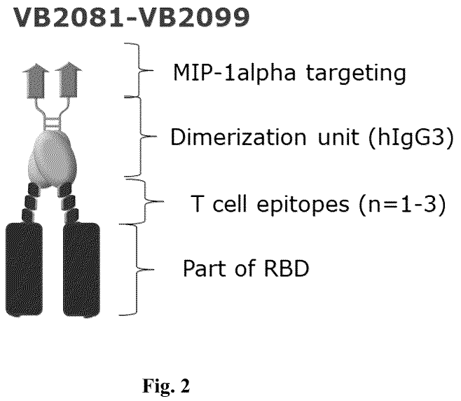

The order and orientation of the above-described units and elements are the same in the multimeric protein, such as in a dimeric protein (illustrated for certain constructs in FIG. 2), and in the polynucleotide.

In the following, the various units and elements of the construct will be discussed in detail. These units and elements are present in the polynucleotide as nucleotide sequences encoding the units while they are present in the polypeptide or multimeric protein as amino acid sequences. For the ease of reading, in the following, the units of the construct are mainly explained in relation to the polypeptide/multimeric/dimeric protein, i.e. on the basis of the amino acid sequences of such constructs.

### Antigenic Unit

There are two primary classes of MHC molecules, MHC class I and MHC II. The terms MHC class I and MHC class II are interchangeably used herein with HLA class I and HLA class II. HLA (human leukocyte antigen) is a major histocompatibility complex in humans. Thus, in some embodiments, the antigenic unit comprises epitopes having a length suitable for specific presentation on MHC class I or MHC class II. In some embodiments, the epitope has a length of from 7 to 11 amino acids for MHC class I presentation. In other embodiments, the epitope has a length of 15 amino acids for MHC class II presentation.

### Subunit Comprising T Cell Epitopes and T Cell Epitope Linkers

The construct of the disclosure comprises an antigenic unit comprising one or more T cell epitopes which are arranged in a subunit of the antigenic unit, and which T cell epitopes are separated from each other by T cell epitope linkers, if more than one T cell epitope is comprised in the subunit.

The one or more T cell epitopes are disease-relevant T cell epitopes, i.e. they are comprised (or naturally found) in proteins of the pathogen which causes the disease or which is involved in causing it, e.g. eggs of a parasite which do not cause a disease but develop into larvae which cause it, and which pathogen is the target of a vaccine comprising the immunogenic construct of the disclosure including such T cell epitopes.

In some embodiments, the antigenic unit comprises one T cell epitope; in other embodiments, the antigenic unit comprises several T cell epitopes, which can be identical or different; preferably the several T cell epitopes are several different T cell epitopes.

In some embodiments, the antigenic unit comprises one or more T cell epitopes of a pathogen, i.e. one T cell epitope of a pathogen or more than one T cell epitope of a pathogen, i.e. multiple T cell epitopes of a pathogen. In some embodiments, the multiple T cell epitopes are of the same pathogen, i.e. (naturally) comprised in the same or different proteins of the pathogen. In other embodiments, the multiple T cell epitopes are of multiple different pathogens, i.e. (naturally) comprised in proteins of different pathogens. In that context, a “different pathogen” may, for example be a different virus or bacterium or a different strain of the same virus or bacterium or it may be the same strain, but comprising one or more mutations.

The construct of the disclosure may be for use in a pan-vaccine, e.g. a vaccine targeting different (seasonal) viruses. For example, the pan-vaccine could target betacoronavirus and influenza or target different strains of e.g. betacoronaviruses or different mutations of the same strain.

In some embodiments, the antigenic unit comprises one or more antigens derived from surface proteins of pathogens, e.g. viral surface proteins such as the spike protein from SARS-CoV-2, hemagglutinin of the influenza virus or gp120 of the HIV virus (human immunodeficiency virus). In some embodiments, the antigenic unit comprises or consist of or more antigens or parts or fragments thereof comprising a Hemagglutinin H1N1 sequence, such as the Hemagglutinin H1N1 sequence set forth in SEQ ID NO: 140.

In other embodiments, the antigen is a full-length protein of a pathogen, preferably a full-length surface protein, e.g. a full-length viral surface protein or bacterial surface protein or a full-length surface protein of any other pathogen. In other embodiments, the antigen is a full-length bacterial protein which is secreted by the bacterium, e.g. secreted into the cytoplasm of infected subjects. In other embodiments, the antigenic unit comprises more than one antigen, i.e. several antigens, each of which being a full-length protein.

Each T cell epitope comprised in the antigenic unit of the construct of the disclosure has a length of from 7 to about 200 amino acids, with the longer T cell epitopes possibly including hotspots of minimal epitopes. A hotspot of minimal epitopes is a region that contains several minimal epitopes (e.g. having a length of from 8-15 amino acids) that are predicted to be presented by different HLA alleles to cover a broad range of world population.

In some embodiments, the antigenic unit comprises T cell epitopes with a length of from 7 to 150 amino acids, preferably of from 7 to 100 amino acids, e.g. from about 10 to about 100 amino acids or from about 15 to about 100 amino acids or from about 20 to about 75 amino acids or from about 25 to about 50 amino acids.

T cell epitopes having a length of about 60 to 200 amino acids may be split into shorter sequences and included into the first antigenic unit separated by the linkers which are described herein. By way of example, a T cell epitope having a length of 150 amino acids may be split into 3 sequences of 50 amino acids each, and included into the first antigenic unit, preferably with a T cell epitope linker separating the 3 sequences from each other.

In some embodiments, the T cell epitope has a length suitable for presentation by MHC (major histocompatibility complex). There are two primary classes of MHC molecules, MHC class I and MHC II. The terms MHC class I and MHC class II are interchangeably used herein with HLA class I and HLA class II. HLA (human leukocyte antigen) is a major histocompatibility complex in humans. Thus, in a preferred embodiment, the antigenic unit comprises T cell epitopes having a length suitable for specific presentation on MHC class I or MHC class II. In some embodiments, the T cell epitope has a length of from 7 to 11 amino acids for MHC class I presentation. In some embodiments, the T cell epitope has a length of from 9 to 60 amino acids, such as from 9 to 30 amino acids, such as 15 to 60 amino acids, such as 15 to 30 amino acids, such as 11 to 15 amino acids, such as 12 to 20 amino acids for MHC class II presentation. In some preferred embodiments the T cell epitope has a length of 15 amino acids for MHC class II presentation.

The T cell epitope may be comprised in any of the pathogen proteins, e.g. surface proteins but also structural and non-structural proteins; in other words, the T cell epitope may be found in the proteins naturally present in said pathogen.

In some embodiments, the T cell epitope is from a conserved region of the pathogen, i.e. conserved between several subgenus, species or strains of respective pathogens. In other words, the T cell epitope may be encoded by a nucleotide sequence which is found in a conserved region of the genome of the pathogen, i.e. conserved between several subgenus, species or strains of respective pathogens. The T cell epitope may thus be conserved between several subgenus, species or strains of respective pathogens, i.e. the amino acid sequence of the T cell epitope is conserved between these.

As an example, the T cell epitope may be from a conserved region of a betacoronavirus, e.g. a region which is conserved between viruses from the same subgenus, such as the subgenus Sarbecovirus, e.g. conserved between SARS-CoV-2, which causes coronavirus disease 2019 (COVID-19) and SARS-CoV, which causes severe acute respiratory syndrome (SARS). By including such T cell epitope in the construct of the disclosure, a vaccine comprising the construct will, or is at least expected to, also provide protection against multiple variants of a betacoronavirus, e.g. variants of SARS-CoV or variants of SARS-CoV-2, which is important for the efficacy of such a vaccine against future variants. Viruses are known to mutate, e.g. undergo viral antigen drift or antigen shift. Finding conserved regions across the genome of betacoronavirus genus indicates that these conserved regions are needed to maintain essential structures or functions, thus it can be assumed that future mutations will take place in the less-conserved regions. By raising an immune response against the conserved regions, the vaccinated individual will be protected also against future variants, or at least is expected to have a higher likelihood of being protected also against future variants.

As an example, the T cell epitope may be from a conserved region of a betacoronavirus, e.g. a region which is conserved between viruses from the same subgenus, such as the subgenus Sarbecovirus, e.g. conserved between SARS-CoV-2, which causes coronavirus disease 2019 (COVID-19) and SARS-CoV, which causes severe acute respiratory syndrome (SARS). By including such T cell epitope in the construct of the disclosure, a vaccine comprising the construct will, or is at least expected to, also provide protection against multiple variants of a betacoronavirus, e.g. variants of SARS-CoV or variants of SARS-CoV-2, which is important for the efficacy of such a vaccine against future variants. Viruses are known to mutate, e.g. undergo viral antigen drift or antigen shift. The presence of conserved regions across the genomes of the betacoronavirus genus indicate that these conserved regions are needed to maintain essential structures or functions. Thus, it can be assumed that future mutations will take place in the less-conserved regions. By raising an immune response against the conserved regions, the vaccinated individual will be protected also against future variants, or at least is expected to have a higher likelihood of being protected also against future variants.

As an example, the T cell epitope may be from a region of a human papilloma virus (HPV), e.g. from HPV16 or HPV18. HPV antigens may be any antigens selected from the list consisting of E1, E2, E6, E7, L1 and L2, e.g. E6 and/or E7 of HPV16 and/or HPV18. By including such T cell epitopes in the construct of the disclosure, a vaccine comprising the construct will provide protection against HPV. HPV infections are involved in certain cancers, such as squamous cell carcinoma of the head and neck, cervical cancer and vulvar squamous cell carcinoma. Indeed, HPV16 viral antigens are expressed in about 50% of all patients with said cancers.

As another example, the T cell epitope may be from a region of a human Influenza virus, such as human Influenza virus A, human Influenza virus B, human Influenza virus C and human Influenza virus D. As an example, the human Influenza virus may be a specific hemagglutinin (HA) subtype, such as H1, H2, and H3, and/or a specific neuraminidase (NA) subtype, such as N1 or N5. As an example, the human Influenza virus may be a H1N1 subtype. By including such T cell epitope in the construct of the disclosure, a vaccine comprising the construct will provide protection against Influenza infection.

In some embodiments, the antigenic unit comprises one part of one antigen. The RBD domain of the spike protein of SARS-CoV-2 or the head or stem domain of hemagglutinin of the influenza virus are examples of parts of an antigen. Sequences of the spike protein and of the RBD domain are available in databases. As an example, in the spike protein of the “Wuhan” strain (NCBI accession number YP_009724390), the RBD sequence is positioned at residues 319 to 542. As another example, hemagglutinin from influenza H1N1, e.g. having SEQ ID NO: 140, can be used.

In yet other embodiments, the antigenic unit comprises several parts of one antigen. In yet other embodiments, the antigenic unit comprises one part of several antigens, e.g. one part of antigen 1 and one part of antigen 2 and 1 part of antigen 3. In yet other embodiments, the antigenic unit comprises several parts of several antigens, e.g. 2 parts of antigen 1 and 3 parts of antigen 2. In some embodiments, the antigenic unit comprises or consist of or more antigens or parts or fragments thereof comprising a RBD sequence of spike protein of SARS-CoV-2 of the Wuhan strain, such as the RBD sequence set forth in SEQ ID NO: 125.

If more than one antigen is comprised in the antigenic unit, or more than 1 part of one or more antigens, the antigens and parts may be separated by a linker. In some embodiments, the linker has similar or the same the properties and/or sequences as the T cell epitope linkers or the dimerization or multimerization units described in this application.

Herein is thus disclosed an immunogenic construct comprising:
(i) a polynucleotide comprising a nucleotide sequence encoding a polypeptide comprising:
   a) a targeting unit targeting or capable of targeting antigen-presenting cells,
   b) a multimerization unit such as a dimerization unit, and
   c) an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
wherein the one or more T cell epitopes and the one or more antigens or parts or fragments thereof are from a pathogen, such as a virus,
wherein the antigenic unit comprises a subunit comprising the T cell epitopes which are separated from each other by T cell epitope linkers, if more than one T cell epitope is comprised in the subunit; and wherein the subunit is connected to the multimerization unit by a unit linker and separated from the one or more antigens or parts or fragments thereof by an antigen linker.

In some embodiments, the immunogenic construct comprises:
(i) a polynucleotide comprising a nucleotide sequence encoding a polypeptide comprising:
   a) a targeting unit targeting or capable of targeting antigen-presenting cells,
   b) a multimerization unit, and
   c) an antigenic unit comprising one or more T cell epitopes and one or more antigens or parts or fragments thereof, or
(ii) a polypeptide encoded by the nucleotide sequence defined in (i); or
(iii) a multimeric protein consisting of multiple polypeptides as defined in (ii);
wherein the one or more T cell epitopes and the one or more antigens or parts or fragments thereof are from a pathogen, such as a virus,
wherein the antigenic unit comprises a subunit comprising the T cell epitopes which are separated from each other by T cell epitope linkers, if more than one T cell epitope is comprised in the subunit; and wherein the subunit is connected to the multimerization unit by a unit linker and separated from the one or more antigens or parts or fragments thereof by an antigen linker.

In some embodiments, the immunogenic construct comprises:
(i) a polynucleotide comprising a nucleotide sequence encoding a polypeptide comprising:
   a) a targeting unit targeting or capable of targeting antigen-presenting cells,
   b) a dimerization unit, and
   c) an antigenic unit comprising one or more T cell epitopes and one or more antigens or parts or fragments thereof, or
(ii) a polypeptide encoded by the nucleotide sequence defined in (i); or
(iii) a dimeric protein consisting of two polypeptides as defined in (ii);
wherein the one or more T cell epitopes and the one or more antigens or parts or fragments thereof are from a pathogen, such as a virus,
wherein the antigenic unit comprises a subunit comprising the T cell epitopes which are separated from each other by T cell epitope linkers, if more than one T cell epitope is comprised in the subunit; and wherein the subunit is connected to the dimerization unit by a unit linker and separated from the one or more antigens or parts or fragments thereof by an antigen linker.

The number of T cell epitopes in the subunit may vary, and depends on the length and number of the other elements included in the antigenic unit, e.g. the T cell epitope linkers, the antigen linker and the one or more antigens or parts or fragments thereof.

In some embodiments, the antigenic unit comprises up to 3500 amino acids, such as from 60 to 3500 amino acids, e.g. from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.

In some embodiments, the subunit comprises 1 to 50 T cell epitopes. In some embodiments, the subunit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes. In other embodiments, the subunit comprises 1 to 3 T cell epitopes or 1 to 5 T cell epitopes or 3 to 6 T cell epitopes or 5 to 15 T cell epitopes or 7 to 17 T cell epitopes or 9 to 19 T cell epitopes.

In some embodiments, the T cell epitopes are randomly arranged in the subunit. In some embodiments, at least one of the following methods for arranging them in the subunit is used to enhance the immune response.

In some embodiments, the T cell epitopes are arranged in the order of more antigenic (immunogenic) to less antigenic (immunogenic) in the direction from the unit linker to the antigen linker. Alternatively, particularly if the hydrophilicity/hydrophobicity varies greatly among the T cell epitopes, it is preferred that the most hydrophobic T cell epitopes is/are positioned substantially in the middle of the subunit and the most hydrophilic T cell epitopes is/are positioned towards the unit linker and/or antigen linker. The skilled person will have no difficulty predicting antigenicity/immunogenicity and/or hydrophilicity/hydrophobicity by methods known in the art and as described herein.

Since a true positioning in the middle of the subunit is only possible if the subunit comprises an odd number of T cell epitopes, the term “substantially” in this context refers to subunits comprising an even number of T cell epitopes, wherein the most hydrophobic T cell epitopes are positioned as close to the middle as possible.

By way of example, a subunit comprises 5 T cell epitopes, which are arranged as follows: 1-2-3*-4-5; with 1, 2, 3*, 4 and 5 each being a different T cell epitope and being a T cell epitope linker and * indicating the most hydrophobic T cell epitope, which is positioned in the middle of the subunit.

In another example, a subunit comprises 6 T cell epitopes, which are arranged as follows: 1-2-3*-4-5-6 or, alternatively, as follows: 1-2-4-3*-5-6; with 1, 2, 3*, 4, 5 and 6 each being a T cell epitope and—being a T cell epitope linker and * indicating the most hydrophobic T cell epitope, which is positioned substantially in the middle of the subunit.

Alternatively, the T cell epitopes may be arranged by alternating a hydrophilic and a hydrophobic T cell epitope. Optionally, GC rich sequences encoding T cell epitopes are arranged in such a way, that GC clusters are avoided. In some embodiments, GC rich sequences encoding T cell epitopes are arranged such that there is at least one non-GC rich T cell sequence between them. GC rich sequences are sequences with a GC content of 60% or more, such as 65% or more, such as 70% or more, such as 75% or more, such as 80% or more.

### T Cell Epitope Linker

When more than one T cell epitope is present in the subunit, the T cell epitopes are separated by T cell epitope linkers. This ensures that each T cell epitope is presented in an optimal way to the immune system.

In some embodiments, the T cell epitope linker is designed to be non-immunogenic. The T cell epitope linker may be a rigid linker, meaning that that it does not allow the two amino acid sequences that it connects to substantially move freely relative to each other. Alternatively, the T cell epitope linker may be a flexible linker, i.e. a linker that allows the two amino acid sequences that it connects to substantially move freely relative to each other.

Both types of linkers are useful. In some embodiments, the T cell epitope linker is a flexible linker, which allows for presenting the T cell epitope in an optimal manner to the immune system, even if the subunit comprises a large number of T cell epitopes.

In some embodiments, the T cell epitope linker is a peptide consisting of from 4 to 40 amino acids, e.g. 35, 30, 25 or 20 amino acids, e.g. from 5 to 20 amino acids or 5 to 15 amino acids or 8 to 20 amino acids or 8 to 15 amino acids 10 to 15 amino acids or 8 to 12 amino acids. In some embodiments, the T cell epitope linker consists of 10 amino acids.

In some embodiments, e.g. in an antigenic unit comprising several T cell epitopes, the T cell epitope linkers comprised in the subunit are identical. If, however, one or more of the T cell epitopes comprises a sequence similar to that of the linker, it may be an advantage to substitute the neighboring T cell epitope linker with a linker of a different sequence. Also, if a T cell epitope/linker junction is predicted to constitute an epitope in itself, then it is preferred to use a T cell epitope linker of a different sequence.

In some embodiments, the T cell epitope linker is a flexible linker, such as a flexible linker which comprises small, non-polar (e.g. glycine, alanine or leucine) or polar (e.g. serine or threonine) amino acids. The small size of these amino acids provides flexibility and allows for mobility of the connected amino acid sequences. The incorporation of serine or threonine can maintain the stability of the linker in aqueous solutions by forming hydrogen bonds with the water molecules, and therefore reduces the unfavorable interaction between the linker and antigens. In some embodiments, the flexible linker is a serine (S) and/or glycine (G) rich linker, i.e. a linker comprising several serine and/or several glycine residues. Preferred examples are GGGGSGGGSS (SEQ ID NO: 19), GGGSG (SEQ ID NO: 20), GGGGS (SEQ ID NO: 21), SGSSGS (SEQ ID NO: 134), GGSGG (SEQ ID NO: 138), or multiple variants thereof such as GGGGSGGGGS (SEQ ID NO: 135), (GGGGS)m (SEQ ID NO: 22), (GGGSS)m (SEQ ID NO: 23), (GGSGG)m (SEQ ID NO: 136), (GGGSG)m (SEQ ID NO: 24), or (SGSSGS)m (SEQ ID NO: 137), where m is an integer from 1 to 5, e.g., 1, 2, 3, 4, or 5. In preferred embodiments, m is 2. In other embodiments, the serine and/or glycine rich linker further comprises at least one leucine (L) residue, such as at least 1 or at least 2 or at least 3 leucine residues, e.g. 1, 2, 3 or 4 leucine residues.

In some embodiments, the T cell epitope linker comprises or consists of LGGGS (SEQ ID NO: 25), GLGGS (SEQ ID NO: 26), GGLGS (SEQ ID NO: 27), GGGLS (SEQ ID NO: 28) or GGGGL (SEQ ID NO: 29). In other embodiments, the T cell epitope linker comprises or consists of LGGSG (SEQ ID NO: 30), GLGSG (SEQ ID NO: 31), GGLSG (SEQ ID NO: 32), GGGLG (SEQ ID NO: 33) or GGGSL (SEQ ID NO: 34). In other embodiments, the T cell epitope linker comprises or consists of LGGSS (SEQ ID NO: 35), GLGSS (SEQ ID NO: 36), or GGLSS (SEQ ID NO: 37).

In other embodiments, the T cell epitope linker comprises or consists of LGLGS (SEQ ID NO: 38), GLGLS (SEQ ID NO: 39), GLLGS (SEQ ID NO: 40), LGGLS (SEQ ID NO: 41), GLGGL (SEQ ID NO: 42) or (GLGGL)m (SEQ ID NO: 90). In other embodiments, the T cell epitope linker comprises or consists of LGLSG (SEQ ID NO: 43), GLLSG (SEQ ID NO: 44), GGLSL (SEQ ID NO: 45), GGLLG (SEQ ID NO: 46) or GLGSL (SEQ ID NO: 47). In other embodiments, the T cell epitope linker comprises or consists of LGLSS (SEQ ID NO: 48), or GGLLS (SEQ ID NO: 49).

In other embodiments, the T cell epitope linker is a serine-glycine linker that has a length of 10 amino acids and comprises 1 or 2 leucine residues.

In some embodiments, the T cell epitope linker comprises or consists of LGGGSGGGGS (SEQ ID NO: 50), GLGGSGGGGS (SEQ ID NO: 51), GGLGSGGGGS (SEQ ID NO: 52), GGGLSGGGGS (SEQ ID NO: 53) or GGGGLGGGGS (SEQ ID NO: 54). In other embodiments, the T cell epitope linker comprises or consists of LGGSGGGGSG (SEQ ID NO: 55), GLGSGGGGSG (SEQ ID NO: 56), GGLSGGGGSG (SEQ ID NO: 57), GGGLGGGGSG (SEQ ID NO: 58) or GGGSLGGGSG (SEQ ID NO: 59). In other embodiments, the T cell epitope linker comprises or consists of LGGSSGGGSS (SEQ ID NO: 60), GLGSSGGGSS (SEQ ID NO: 61), GGLSSGGGSS (SEQ ID NO: 62), GGGLSGGGSS (SEQ ID NO: 63) or GGGSLGGGSS (SEQ ID NO: 64).

In further embodiments, the T cell epitope linker comprises or consists of LGGGSLGGGS (SEQ ID NO: 65), GLGGSGLGGS (SEQ ID NO: 66), GGLGSGGLGS (SEQ ID NO: 67), GGGLSGGGLS (SEQ ID NO: 68) or GGGGLGGGGL (SEQ ID NO: 69). In other embodiments, the T cell epitope linker comprises or consists of LGGSGLGGSG (SEQ ID NO: 70), GLGSGGLGSG (SEQ ID NO: 71), GGLSGGGLSG (SEQ ID NO: 72), GGGLGGGGLG (SEQ ID NO: 73) or GGGSLGGGSL (SEQ ID NO: 74). In yet other embodiments, the T cell epitope linker comprises or consists of LGGSSLGGSS (SEQ ID NO: 75), GLGSSGLGSS (SEQ ID NO: 76), or GGLSSGGLSS (SEQ ID NO: 77).

In other embodiments, the T cell epitope linker comprises or consists of GSGGGA (SEQ ID NO: 91), GSGGGAGSGGGA (SEQ ID NO: 92), GSGGGAGSGGGAGSGGGA (SEQ ID NO: 93), GSGGGAGSGGGAGSGGGAGSGGGA (SEQ ID NO: 94) or GENLYFQSGG (SEQ ID NO: 95). In yet other embodiments, the T cell epitope linker comprises or consists of SGGGSSGGGS (SEQ ID NO: 96), GGGGSGGGGS (SEQ ID NO: 97), SSGGGSSGGG (SEQ ID NO: 98), GGSGGGGSGG (SEQ ID NO: 99), GSGSGSGSGS (SEQID NO: 100), GGGSSGGGSG (SEQ ID NO: 87, corresponding to amino acids 121-130 of SEQ ID NO: 1), GGGSSS (SEQ ID NO: 101), GGGSSGGGSSGGGSS (SEQ ID NO: 102) or GLGGLAAA (SEQ ID NO: 103).

In other embodiments, the T cell epitope linker is a rigid linker. Such rigid linkers may be useful to efficiently separate (larger) antigens and prevent their interferences with each other. In some embodiments, the T cell epitope linker comprises or consist of KPEPKPAPAPKP (SEQ ID NO: 104), AEAAAKEAAAKA (SEQ ID NO: 105), (EAAAK)m (SEQ ID NO: 106), PSRLEEELRRRLTEP (SEQ ID NO: 107) or SACYCELS (SEQ ID NO: 108).

In other embodiments, the T cell epitope linker comprises or consists of the sequence TQKSLSLSPGKGLGGL (SEQ ID NO: 78). In other embodiments, the T cell epitope linker comprises or consists of the sequence SLSLSPGKGLGGL (SEQ ID NO: 79). In other embodiments, the T cell epitope linker comprises or consists of AAY or GPGPG (SEQ ID NO: 139).

In yet other embodiments, the T cell epitope linker is a GSAT linker, i.e. a linker comprising one or more glycine, serine, alanine and threonine residues, e.g. a linker comprising or consisting of the sequence GGSAGGSGSGSSGGSSGASGTGTAGGTGSGSGTGSG (SEQ ID NO: 80); or the T cell epitope is a SEG linker, i.e. a linker comprising one or more serine, glutamic acid and glycine residues, e.g. a linker comprising or consisting of the sequence GGSGGGSEGGGSEGGGSEGGGSEGGGSEGGGSGGGS (SEQ ID NO: 81), or the T cell epitope linker is ELKTPLGDTTHT (SEQ ID NO: 124; corresponding to amino acids 94-105 of SEQ ID NO: 1).

In other embodiments, the T cell epitope linker is a cleavable linker, e.g. a linker which includes one or more recognition sites for endopeptidases, e.g. endopeptidases such as furin, caspases, cathepsins and the like. Cleavable linkers may be introduced to release free functional protein domains (e.g. encoded by larger antigens), which may overcome steric hindrance between such domains or other drawbacks due to interference of such domains, like decreased bioactivity, altered biodistribution.

Examples of T cell epitope linkers are disclosed in paragraphs [0098]-[0099] and in the recited sequences of WO 2020/176797A1 (in particular SEQ ID NOs: 37 to 65 and SEQ ID NOs: 67 to 76), which is incorporated herein by reference and in paragraphs [0135] to [0139] of US 2019/0022202A1, which is incorporated herein by reference.

### T Cell Epitopes

The present constructs comprise, as described herein in detail, one or more T cell epitopes. Preferably, the T cell epitopes are from a pathogen, for example from a virus or a pathogenic bacteria.

In some embodiments, the T cell epitopes are known to be immunogenic, e.g. their immunogenicity has been confirmed by appropriate methods and the results have been published, e.g. in a scientific publication.

For example, useful T cell epitopes against infection by SARS-CoV2 in humans can be found in Grifoni et al., 2021 (Cell Host Microbe. 2021 Jul. 14; 29(7): 1076-1092). Such T cell epitopes may thus be included in the present constructs to provide protection against SARS-CoV2 in humans.

Another example of such T cell epitopes is the T cell epitope with the sequence CTELKLSDY (SEQ ID NO: 82) of the nucleoprotein from Influenza A virus, which has been studied for immune reactivity in 39 publications, tested in 54 T cell assays and 34 MHC ligand assays. Other examples include the T cell epitope with the sequence NLVPMVATV (SEQ ID NO: 83) of the 65 kDa phosphoprotein from human herpesvirus 5 (human cytomegalovirus) which has been studied for immune reactivity in 327 publications, tested in 754 T cell assays, 25 B cell assays and 52 MHC ligand assays and the T cell epitope with the sequence KLVANNTRL (SEQ ID NO: 84) of diacylglycerol acyltransferase/mycolyltransferase Ag85B from Mycobacterium tuberculosis, which has been studied for immune reactivity in 13 publications, tested in 34 T cell assays, 4 B cell assays and 26 MHC ligand assays (source: immune epitope database and analysis resource (www.iedb.org), with link to publications available therein).

In other embodiments, the T cell epitopes are selected based on their predicted ability to bind to HLA class I/II alleles, i.e. selected in silico based on predictive HLA-binding algorithms. After having identified relevant epitopes, the epitopes are ranked according to their ability or predicted ability to bind to HLA class I/II alleles and the epitopes that are predicted to bind best are selected to be included in the antigenic unit.

Any suitable HLA-binding algorithm may be used, such as one of the following:

Available software analysis of peptide-MHC binding (IEDB, NetMHCpan and NetMHCIIpan) may be downloaded or used online from the following websites:

www.iedb.org/
services.healthtech.dtu.dk/service.php?NetMHCpan-4.0
services.healthtech.dtu.dk/service.php?NetMHCIIpan-3.2

Commercially available advanced software to predict optimal sequences for vaccine design are found here:

www.oncoimmunity.com/
omictools.com/t-cell-epitopes-category
github.com/griffithlab/pVAC-Seq
crdd.osdd.net/raghava/cancertope/help.php
www.epivax.com/tag/neoantigen/

In other embodiments, each T cell epitope is ranked with respect to its predicted binding affinity and/or antigenicity or immunogenicity, and the predicted most antigenic or most immunogenic epitopes are selected and preferably optimally arranged in the antigenic unit.

Conserved T cell epitopes for inclusion into the antigenic unit may be identified by the method below which is explained using a betacoronavirus as an example, but which may be used for the identification of conserved T cell epitopes of other viruses or other pathogens.

For identifying T cell epitopes that are conserved between SARS-CoV-2 and SARS-CoV, the following steps are carried out:
1) Identification of sets of HLA class I and II alleles that are specific for a subset of the population or a defined ethnic group or a defined geographic region
2) Identification of genomic regions in the conserved viral sequence of SARS-CoV-2 that contain hotspots of minimal epitopes, i.e. minimal epitopes predicted to be presented by different HLA class I and II alleles to cover a broad range of the world's population
3) Selection of SARS-CoV-2 T cell epitopes in the hotspots that cover the highest number of different HLA class I and II alleles
4) From the selected T cell epitopes, identifying those that are conserved between SARS-CoV and SARS-CoV-2
5) Checking the selected T cell epitopes for similarity to sequences found in the normal human proteome and removing those T cell epitopes with a high number of matches to such sequences
6) From the remaining selected T cell epitopes, identifying those that match or have a high similarity to minimal epitopes already described to be immunogenic

Example 1 shows how such T cell epitopes were identified from conserved regions of SARS-CoV2.

### Antigens or Parts or Fragments Thereof

The construct of the disclosure comprises an antigenic unit comprising one or more antigens or parts or fragments thereof, which are separated from the subunit comprising the one or more T cell epitopes by the antigen linker. In some embodiments, the parts or fragments of an antigen are immunogenic.

The antigen linker is designed to be non-immunogenic and may be a flexible linker, which allows for correct folding of the antigen such that the conformational B cell epitopes are presented to the immune system. Protein modelling may be used to model 3D structures/conformations of the antigen connected to an antigen linker to determine which length and amino acid sequence promotes correct folding, and to suggest appropriate linkers enabling conformationally correct folding.

Typically, the antigen linker is a peptide consisting of from 10 to 80 amino acids, e.g. from 11 to 70 amino acids or 15 to 60 amino acids or 20 to 50 amino acids or 25 to 45 amino acids or 12 to 45 amino acids or 13 to 40 amino acids or 30 to 40 amino acids.

In some embodiments, the antigen linker is a serine (S) and/or glycine (G) rich linker, which may comprise one or more leucines, e.g. like the T cell epitopes linkers described above.

In other embodiments, the antigen linker comprises or consists of the sequence TQKSLSLSPGKGLGGL (SEQ ID NO: 78). In other embodiments, the antigen linker comprises or consists of the sequence SLSLSPGKGLGGL (SEQ ID NO: 79).

In other embodiments, the antigen linker is a GSAT linker, i.e. a linker comprising one or more glycine, serine, alanine and threonine residues, e.g. a linker comprising or consisting of the sequence GGSAGGSGSGSSGGSSGASGTGTAGGTGSGSGTGSG (SEQ ID NO: 80); or the antigen linker is a SEG linker, i.e. a linker comprising one or more serine, glutamic acid and glycine residues, e.g. a linker comprising or consisting of the sequence GGSGGGSEGGGSEGGGSEGGGSEGGGSEGGGSGGGS (SEQ ID NO: 81).

Examples of antigen linkers are disclosed in paragraphs [0098]-[0099] and in the recited sequences of WO 2020/176797A1 (in particular SEQ ID NOs: 37 to 65 and SEQ ID NOs: 67 to 76), which is incorporated herein by reference and in paragraphs [0135] to [0139] of US 2019/0022202A1, which is incorporated herein by reference.

The one or more antigens or parts or fragments thereof are disease-relevant antigens, i.e. they are comprised (or naturally found) in proteins of the pathogen which causes the disease or is involved in causing it, and which is the target of a vaccine comprising the immunogenic construct of the disclosure which includes such antigens.

The one or more antigens or parts or fragments thereof comprise conformational B ell epitopes, but may also comprise linear B cell epitopes and/or T cell epitopes. In contrary to the T cell epitopes in the subunit, these T cell epitopes are not isolated, but are presented to the immune system in their natural environment, i.e. flanked by the amino acid residues which are present in the antigen.

In some embodiments, the antigenic unit comprises one or more antigens derived from surface proteins of pathogens, e.g. viral surface proteins such as the spike protein from SARS-CoV-2, hemagglutinin of the influenza virus or gp120 of the HIV virus (human immunodeficiency virus). In other words, the antigenic unit in such embodiments comprises one or more antigens, parts or fragments thereof, which are naturally found in surface proteins of pathogens.

In other embodiments, the antigen is a full-length protein of a pathogen, preferably a full-length surface protein, e.g. a full-length viral surface protein or bacterial surface protein or a full-length surface protein of any other pathogen. In other embodiments, the antigen is a full-length bacterial protein which is secreted by the bacterium, e.g. secreted into the cytoplasm of infected subjects. In other embodiments, the antigenic unit comprises more than one antigen, i.e. several antigens, each of which being a full-length protein.

In other embodiments, the antigenic unit comprises one part or fragment of one antigen. The RBD domain of the spike protein of SARS-CoV-2 or the head or stem domain of hemagglutinin of the influenza virus are examples of parts or fragments of an antigen. Sequences of the spike protein and of the RBD domain are available in databases. As an example, in the spike protein of the “Wuhan” strain (NCBI accession number YP_009724390), the RBD sequence is positioned at residues 319 to 542. As another example, hemagglutinin from influenza H1N1, e.g. having SEQ ID NO: 140, can be used.

In other embodiments, the antigenic unit comprises several parts or fragments of one antigen. In other embodiments, the antigenic unit comprises one part or fragment of several antigens, e.g. one part of antigen 1 and one part of antigen 2 and 1 part of antigen 3. In other embodiments, the antigenic unit comprises several parts or fragments of several antigens, e.g. 2 parts of antigen 1 and 3 parts of antigen 2.

If more than one antigen is comprised in the antigenic unit, or more than 1 part of one or more antigens, the antigens and parts thereof may be separated by a linker. In some embodiments, the linker has the same properties and/or sequences as the T cell epitope linkers or the antigen linkers described elsewhere in this application.

### Unit Linker

The antigenic unit is connected to the multimerization unit, such as a dimerization unit, by a unit linker. The unit linker is preferably non-immunogenic.

The unit linker may comprise a restriction site in order to facilitate the construction of the polynucleotide. In a preferred embodiment, the unit linker is GLGGL (SEQ ID NO: 42) or GLSGL (SEQ ID NO: 85). In some embodiments, the unit linker comprises or consists of GGGGS (SEQ ID NO: 21), GGGGSGGGGS (SEQ ID NO: 97), (GGGGS)m (SEQ ID NO: 109), EAAAK (SEQ ID NO: 110), (EAAAK)m (SEQ ID NO: 111), (EAAKG)mS (SEQ ID NO: 112) where m is an integer greater than or equal to 1, GPSRLEEELRRRLTEPG (SEQ ID NO: 113), AAY or HEYGAEALERAG (SEQ ID NO: 114).

### Dimerization/multimerization Unit

The construct of the disclosure comprises a multimerization unit, such as a dimerization unit.

### Multimerization Unit

The term “multimerization unit” as used herein, refers to a sequence of nucleotides or amino acids between the antigenic unit and the targeting unit. In addition to connecting the antigenic unit and the targeting unit, the multimerization unit facilitates multimerization of/joins multiple polypeptides, such as two, three, four or more polypeptides, into a multimeric protein, such as a dimeric protein, a trimeric protein or a tetrameric protein. Furthermore, the multimerization unit also provides the flexibility in the multimeric protein to allow optimal binding of the targeting unit to the surface molecules on the APCs, even if they are located at variable distances. The multimerization unit may be any unit that fulfils one or more of these requirements.

In some embodiments, the multimerization unit is a trimerization unit, such as a collagen-derived trimerization unit, such as a human collagen-derived trimerization domain, such as human collagen derived XVIII trimerization domain (see for instance A. Alvarez-Cienfuegos et al., Sci Rep 6, 28643 (2016)) or human collagen XV trimerization domain. Thus, in some embodiments, the multimerization unit is a trimerization unit that comprises or consists of the nucleotide sequence with SEQ ID NO: 126, or an amino acid sequence encoded by said nucleotide sequence. In other embodiments, the trimerization unit is the C-terminal domain of T4 fibritin. Thus, in some embodiments, the multimerization unit is a trimerization unit that comprises or consists of the amino acid sequence with SEQ ID NO: 127, or a nucleotide sequence encoding said amino acid sequence.

In other embodiments, the multimerization unit is a tetramerization unit, such as a domain derived from p53, optionally further comprising a hinge region as described below. Thus, in one embodiment, the multimerization unit is a tetramerization unit that comprises or consists of the nucleotide sequence with SEQ ID NO: 128, or an amino acid sequence encoded by said nucleotide sequence, optionally further comprising a hinge region as described below.

The term “hinge region” in the context of a multimerization unit refers to an amino acid sequence comprised in the multimerization unit that contributes to joining two or more of the polypeptides, e.g. three or four polypeptides, i.e. contributes to the formation of the multimeric or dimeric protein and/or functions as a flexible spacer, allowing the targeting units of the multimeric protein to bind simultaneously to multiple surface molecules on APCs, even if these surface molecules are located at variable distances.

### Dimerization Unit

The term “dimerization unit” as used herein, refers to a sequence of nucleotides or amino acids between the antigenic unit and the targeting unit. In addition to connecting the antigenic unit and the targeting unit, the dimerization unit facilitates dimerization of/joins two polypeptides into a dimeric protein. Furthermore, the dimerization unit also provides the flexibility in the dimeric protein to allow optimal binding of the targeting unit to the surface molecules on the APCs, even if they are located at variable distances. The dimerization unit may be any unit that fulfils one or more of these requirements.

Accordingly, in some embodiments the construct comprises a dimerization unit comprising a hinge region. In other embodiments, the dimerization unit comprises a hinge region and another domain that facilitates dimerization. In other embodiments, the dimerization unit comprises a hinge region, a dimerization unit linker and another domain that facilitates dimerization, wherein the dimerization unit linker connects the hinge region to the other domain that facilitates dimerization. The dimerization unit linker is further described below.

In some embodiments, the dimerization unit linker is a glycine-serine rich linker, preferably GGGSSGGGSG (SEQ ID NO: 87), i.e. the dimerization unit comprises a glycine-serine rich dimerization unit linker and preferably the dimerization unit linker GGGSSGGGSG (SEQ ID NO: 87).

The term “hinge region” refers to an amino acid sequence comprised in the dimerization unit that contributes to joining two of the polypeptides, i.e. contributes to the formation of the dimeric protein. In the context of multimerization units that facilitate multimerization of/joins more than two polypeptides, the term “hinge region” refers to an amino acid sequence comprised in such multimerization unit that contributes to joining more than two polypeptides, e.g. three or four polypeptides.

Moreover, the hinge region functions as a flexible spacer, allowing the two targeting units of the dimeric protein to bind simultaneously to two surface molecules on APCs, even if they are located at variable distances. The hinge region may be Ig derived, such as derived from IgG, e.g. IgG1, IgG2 or IgG3. In some embodiments, the hinge region is derived from IgM, e.g. comprising or consisting of the nucleotide sequence with SEQ ID NO: 117 or an amino acid sequence encoded by said nucleotide sequence. The hinge region may contribute to the dimerization (or multimerization) through the formation of covalent bond(s), e.g. disulfide bridge(s) between cysteine residues. Thus, in some embodiments, the hinge region has the ability to form one or more covalent bonds. Preferably, the covalent bond is a disulfide bridge.

In some embodiments, the dimerization unit comprises or consists of a hinge exon h1 and hinge exon h4 (human hinge region 1 and human hinge region 4) having an amino acid sequence having at least 80% sequence identity to the amino acid sequence 94-120 of SEQ ID NO: 1.

In preferred embodiments, the dimerization unit comprises or consists of a hinge exon h1 and a hinge exon h4 with an amino acid sequence having at least 85% sequence identity to the amino acid sequence 94-120 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In some embodiments, the dimerization unit comprises or consists of a hinge exon h1 and a hinge exon h4 with the amino acid sequence 94-120 of SEQ ID NO: 1, or a nucleotide sequence encoding the amino acid sequence.

In some embodiments, the dimerization unit comprises or consists of the amino acid sequence 94-120 of SEQ ID NO: 1, except that at the most four amino acids have been substituted, deleted or inserted, such as at the most three amino acids, such as at the most two amino acids or such as at the most one amino acid.

In some embodiments, the dimerization unit comprises or consists of a nucleic acid sequence having at least 80% sequence identity to the nucleotide sequence with SEQ ID NO: 118.

In further embodiments, the dimerization unit comprises or consists of a nucleotide sequence having at least 85% sequence identity to the nucleotide sequence with SEQ ID NO: 118, such as at least 86% or at least 87%, 88%, 89%, 90%, 91%, 92%, 93%, 94%, 95%, 96%, 97%, 98% or at least 99% sequence identity.

In yet further preferred embodiments, the dimerization unit comprises or consists of the nucleotide sequence of SEQ ID NO: 118.

In other embodiments, the dimerization unit comprises another domain that facilitates dimerization; preferably, said other domain is an immunoglobulin domain, such as an immunoglobulin constant domain (C domain), such as a CH1 domain, a CH2 domain or a carboxyterminal C domain (i.e. a CH3 domain), or a sequence that is substantially identical to such C domains or a variant thereof. Preferably, the other domain that facilitates dimerization is a carboxyterminal C domain derived from IgG. More preferably, the other domain that facilitates dimerization is a carboxyterminal C domain derived from IgG3.

In some embodiments, the dimerization unit comprises or consists of a carboxyterminal C domain derived from IgG3 with an amino acid sequence having at least 80% sequence identity to the amino acid sequence 131-237 of SEQ ID NO: 1, or a nucleotide sequence encoding the amino acid sequence.

In preferred embodiments, the dimerization unit comprises or consists of a carboxyterminal C domain derived from IgG3 with an amino acid sequence having at least 85% sequence identity to the amino acid sequence 131-237 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In preferred embodiments, the dimerization unit comprises or consists of a carboxyterminal C domain derived from IgG3 with the amino acid sequence 131-237 of SEQ ID NO: 1.

In preferred embodiments, the dimerization unit comprises or consists of the amino acid sequence 131-237 of SEQ ID NO: 1, except that at the most 16 amino acids have been substituted, deleted or inserted, such as at the most 15, 14, 13, 12, 11, 10, 9, 8, 7, 6, 5, 4, 3, 2, or 1 amino acid.

In other preferred embodiments, the dimerization unit comprises or consists of a nucleotide sequence having at least 80% sequence identity to the nucleotide sequence with SEQ ID NO: 119.

In further embodiments, the dimerization unit comprises or consists of a nucleotide sequence having at least 85% sequence identity to the nucleotide sequence with SEQ ID NO: 119, such as at least 86% or at least 87%, 88%, 89%, 90%, 91%, 92%, 93%, 94%, 95%, 96%, 97%, 98% or at least 99% sequence identity. In yet further embodiments, the dimerization unit comprises or consists of the nucleotide sequence of SEQ ID NO: 119.

The immunoglobulin domain contributes to dimerization through non-covalent interactions, e.g. hydrophobic interactions. Thus, in one embodiment, the immunoglobulin domain has the ability to form dimers via noncovalent interactions. Preferably, the noncovalent interactions are hydrophobic interactions.

It is preferred that if the dimerization unit comprises a CH3 domain, it does not comprise a CH2 domain and vice versa.

In preferred embodiments, the dimerization unit comprises a hinge exon h1, a hinge exon h4, a dimerization unit linker and a CH3 domain of human IgG3. In further preferred embodiments, the dimerization unit comprises a polypeptide consisting of hinge exon h1, hinge exon h4, a dimerization unit linker and a CH3 domain of human IgG3.

In other preferred embodiments, the dimerization unit consists of a polypeptide consisting of hinge exon h1, hinge exon h4, a dimerization unit linker and a CH3 domain of human IgG3.

In some embodiments, the dimerization unit comprises an amino acid sequence having at least 80% sequence identity to the amino acid sequence 94-237 of SEQ ID NO: 1.

In preferred embodiments, the dimerization unit comprises an amino acid sequence having at least 85% sequence identity to the amino acid sequence 94-237 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In more preferred embodiments the dimerization unit consists of an amino acid sequence having at least 80% sequence identity to the amino acid sequence 94-237 of SEQ ID NO: 1, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In some embodiments, the dimerization unit consists of the amino acid sequence 94-237 of SEQ ID NO: 1, or a nucleotide sequence encoding the amino acid sequence.

In some embodiments, the dimerization unit comprises the amino acid sequence 94-237 of SEQ ID NO: 1, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the dimerization unit consists of the amino acid sequence 94-237 of SEQ ID NO: 1, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In some embodiments, the dimerization unit comprises a nucleotide sequence having at least 80% sequence identity to the nucleotide sequence with SEQ ID NO: 120.

In further embodiments, the dimerization unit comprises a nucleotide sequence having at least 85% sequence identity to the nucleotide sequence with SEQ ID NO: 120, such as at least 86% or at least 87%, 88%, 89%, 90%, 91%, 92%, 93%, 94%, 95%, 96%, 97%, 98% or at least 99% sequence identity. In yet further embodiments, the dimerization unit comprises the nucleotide sequence of SEQ ID NO: 120.

In other embodiments, the dimerization unit consists of a nucleotide sequence having at least 80% sequence identity to the nucleotide sequence with SEQ ID NO: 120.

In further embodiments, the dimerization unit consists of a nucleotide sequence having at least 85% sequence identity to the nucleotide sequence with SEQ ID NO: 120, such as at least 86% or at least 87%, 88%, 89%, 90%, 91%, 92%, 93%, 94%, 95%, 96%, 97%, 98% or at least 99% sequence identity.

In yet further embodiments, the dimerization unit has the nucleotide sequence of SEQ ID NO: 120.

In one embodiment, the dimerization unit comprises or consists of the dHLX protein, e.g. the dHLX protein comprising or consisting of the nucleotide sequence with SEQ ID NO: 121 or the amino acid sequence encoded by said nucleotide sequence.

In some embodiments, the construct comprises a dimerization unit linker, which preferably is a glycine-serine rich linker, preferably GGGSSGGGSG (SEQ ID NO: 87), i.e. the dimerization unit comprises a glycine-serine rich dimerization unit linker and preferably the dimerization unit linker GGGSSGGGSG (SEQ ID NO: 87).

### Targeting Unit

The construct of the disclosure comprises a targeting unit that targets or is capable of targeting antigen-presenting cells (APCs).

The term “targeting unit” as used herein refers to a unit that delivers the construct of the disclosure, more specifically the polypeptide (multimeric or dimeric protein; encoded by the polynucleotides described herein), to an antigen-presenting cell for MHC class II-restricted presentation to CD4+ T cells or for providing cross presentation to CD8+ T cells by MHC class I restriction.

APCs include dendritic cells (DCs) and subsets thereof.

Due to the targeting unit, the construct of the disclosure attracts DCs, neutrophils and other immune cells. Thus, the construct will not only target the antigenic unit comprised therein to specific cells, but in addition facilitate a response-amplifying effect (adjuvant effect) by recruiting specific immune cells to the administration site of a vaccine comprising the construct or a polynucleotide encoding it. This unique mechanism is of great importance in a clinical setting, since the construct can be administered to a subject in the form of a vaccine, which does not need to comprise any adjuvants, since the construct comprised in the vaccine provides the adjuvant effect.

The targeting unit is designed to target the construct of the disclosure to surface molecules expressed on the APCs, such as molecules expressed exclusively on subsets of DCs.

Examples of such surface molecules on APCs are HLA, cluster of differentiation 14 (CD14), cluster of differentiation 40 (CD40), CLEC9A, chemokine receptors and Toll-like receptors (TLRs). Chemokine receptors include C-C motif chemokine receptor 1 (CCR1), C-C motif chemokine receptor 3 (CCR3), C-C motif chemokine receptor 4 (CCR4), C-C motif chemokine receptor 5 (CCR5), C-C motif chemokine receptor 6 (CCR6), C-C motif chemokine receptor 7 (CCR7), C-C motif chemokine receptor 8 (CCR8) and XCR1. Toll-like receptors include TLR-2, TLR-4 and TLR-5.

The targeting unit is or comprises a moiety that interacts with these surface molecules.

Thus, the targeting unit may comprise or consist of an antibody-binding region, such as the antibody variable domains (VL and VH), with specificity for HLA, CD14, CD40, CLEC9A or Toll-like receptors. In other embodiments, the targeting unit comprises or consists of a synthetic or natural ligand. Examples include soluble CD40 ligand, natural ligands like chemokines, for example in their human forms, e.g. chemokine ligand 5, also called C-C motif ligand 5 (CCL5 or RANTES), macrophage inflammatory protein alpha and its isoforms, including mouse CCL3 (or MIP-1α), and human isoforms hCCL3, hCCL3L1, hCCL3L2 and hCCL3L3, chemokine ligand 4 (CCL4) and its isoform CCL4L, chemokine ligand 19 (CCL19), chemokine ligand 20 (CCL20), chemokine ligand 21 (CCL21), chemokine motif ligand 1 or 2 (XCL1 or XCL2) and bacterial antigens like for example flagellin.

In some embodiments, the targeting unit has affinity for an MHC class II protein. Thus, in some embodiments, the targeting unit comprises or consists of an antibody-binding region, such as the antibody variable domains (VL and VH) with specificity for MHC class II proteins, for example selected from the group consisting of anti-HLA-DP, anti-HLA-DR and anti-pan HLA class II.

In other embodiments, the targeting unit has affinity for a surface molecule selected from the group consisting of CD14, CD40, TLR-2, TLR-4 and TLR-5. Thus, in one embodiment the targeting unit comprises or consist of an antibody-binding region such as the antibody variable domains (VL and VH) with specificity for CD14, CD40, TLR-2, TLR4 or TLR-5, such as anti-CD14, anti-CD40, anti-TLR-2, anti-TLR-4 or anti-TLR-5. In yet another embodiment, the targeting unit comprises or consists of flagellin, which has affinity for TLR-5. In other embodiments, the targeting unit comprises or consists of an antibody-binding region with specificity for CLEC9A, such as anti-CLEC9A or variants thereof, such as anti-CLEC9A Fv or the targeting unit comprises or consists of a CLEC9 ligand, e.g. a CLEC9 ligand encoded by the nucleotide sequence with SEQ ID NO: 115.

Preferably, the targeting unit has affinity for a chemokine receptor selected from CCR1, CCR3, CCR4, CCR5, CCR6, CCR7 and CCR8, more preferably for a chemokine receptor selected from CCR1, CCR3 and CCR5. In some embodiments, the targeting unit has affinity for the chemokine receptor CCR7. In other embodiments, the targeting unit comprises or consists of CCL19 (e.g. comprising or consisting of a nucleotide sequence of SEQ ID NO: 116 or comprising or consisting of an amino acid sequence encoded by SEQ ID NO: 116) or CCL21, such as the human forms of CCL19 or CCL21.

Preferably, the targeting comprises or consists of chemokine human macrophage inflammatory protein alpha (human MIP1-α (hMIP-1α), also called LD78β or CCL3L1), and its isoforms, including mouse (CCL3 or MIP-1α), and human isoforms hCCL3, hCCL3L1, hCCL3L2 and hCCL3L3, chemokine ligand 4 (CCL4) and its isoform CCL4L, chemokine ligand 19 (CCL19), chemokine ligand 20 (CCL20), chemokine ligand 21 (CCL21), which binds to its cognate receptors, CCR1, CCR3 and CCR5 expressed on the cell surface of APCs.

In other embodiments, the targeting unit is an antibody-binding region with specificity for a dendritic cell receptor selected from the group CLEC9A, CD11c, CD80, CD86, CD141, CD172a, CD11b, CD103, CD83, CD14, CD206, CD303 and CD85g.

In other embodiments, the targeting unit comprises or is a ligand chosen from the table below of attracting ligands.

| Ligand | DC receptors |
|---|---|
| CCL3L1 | CCR1, CCR3, CCR5 |
| CCL3 | CCR1, CCR4, CCR5 |
| CCL5 | CCR1, CCR4, CCR5, CCR3 |
| CCL7 | CCR1, CCR3 |
| CCL20 | CCR6 |
| CCL4 | CCR4, CCR5 |
| CCL22 | CCR4 |
| XCL1 | XCR1 |
| XCL2 | XCR1 |
| CCL17 | CCR4 |
| CCL23 | CCR1 |
| CCL2 | CCR2, CCR4 |
| CX3CL1 | CX3CR1 |
| CCL19 | CCR7 |
| CCL21 | CCR7 |
| CXCL12 | CXCR4 |
| CXCL13 | CXCR5 |
| CCL11 | CCR3 |
| CCL26 | CCR3 |
| CCL13 | CCR3 |
| CCL3L1 | CCR5 |
| CCL27 | CCR10 |
| CCL28 | CCR10 |
| CCL25 | CCR9 |
| CXCL9 | CXCR3A, CXCR3B |
| CXCL10 | CXCR3A, CXCR3B |
| CXCL11 | CXCR3A, CXCR3B |
| CXCL4 | CXCR3B |

In other embodiments, the targeting unit comprises or is a ligand chosen from the table below.

| Ligand | DC receptors |
|---|---|
| FLT3L | FLT3 |
| CD40L | CD40 |
| 4-1BBL | 4-1BB |
| mAb | CD205 |
| GM-CSF | GM-CSFR |
| CD27 | CD70 |
| RANKL | RANK |
| ICOS | ICOSL |
| HMGB1 | RAGE, TLR4, TLR9, TIM-3 |
| Annexin A1 | FPR |
| HSPs | SREC1, LOX1, CD91 |
| PGRS33 | TLR2 |
| PGRS 17/11 | TLR2 |
| Flagellin | TLR5 |
| Hemagglutinin | TLR2 |
| IL33 | IL-1R1 |
| Porins | TLR2 |

In other embodiments, the targeting unit is capable of targeting a receptor selected from the group consisting of P2Y2, TLR2, TLR8, P2X7, TLR1, TLR11, TLR2, TLR3, TLR4, TLR6, TLR6/2, TLR7 and TLR9. In more specific embodiments, such receptors could be targeted by antibody-binding regions with specificity for any of the receptors, an activating mAbs or a molecule such as MyD88.

In other embodiments, the targeting unit is capable of degrading STAT3. In specific embodiments, antibody mimetics (monobodies) are activated upon internalization and will trigger the degradation of STAT3 inside the APCs.

The binding of the targeting unit to its cognate receptors leads to internalization of the construct into the APC and degradation of the protein into small peptides that are loaded onto MHC molecules and presented to CD4+ and CD8+ T cells to induce specific immune responses. Peptides loaded onto MHC class II molecules can be recognized by antigen-specific CD4+ T helper cells, whereas peptides loaded on MHC class I molecules can be recognized by antigen-specific CD8+ T cells, leading to proliferation and activation of cytotoxic function. Presentation of internalized antigens on MHC II molecules is a process termed cross-presentation. Once stimulated, and with help from activated CD4+ T cells, CD8+ T cells will target and kill cells expressing the same antigens.

In some embodiments, the targeting unit is MIP-1α, preferably hMIP-1α. Not only does MIP-1α attract APCs to the construct through its chemotactic ability, it also causes internalization of the construct through both the classical and cross-presentation pathway, whereby the epitopes are processed by enzymes and presented on the cell surface to raise the T cell response, particularly Th1 CD4+ T cell responses and CD8+ T cell responses. MIP-1α is also capable of supporting the induction of antibody responses, in particular IgG2a, which is important for protection against infection.

In preferred embodiments, the targeting unit comprises an amino acid sequence having at least 80% sequence identity to the amino acid sequence 24-93 of SEQ ID NO: 1, such as comprising the amino acid sequence 26-93 of SEQ ID NO: 1 or comprising the amino acid sequence 28-93 of SEQ ID NO: 1.

In some preferred embodiments, the targeting unit comprises an amino acid sequence having at least 85% sequence identity to the amino acid sequence 24-93 of SEQ ID NO: 1, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the amino acid sequence 24-93 of SEQ ID NO: 1.

In other preferred embodiments, the targeting unit consists of an amino acid sequence having at least 80% sequence identity to the amino acid sequence 24-93 of SEQ ID NO: 1, such as consisting of the amino acid sequence 26-93 of SEQ ID NO: 1 or comprising the amino acid sequence 28-93 of SEQ ID NO: 1.

In other preferred embodiments, the targeting unit consists of an amino acid sequence having at least 85% sequence identity to the amino acid sequence 24-93 of SEQ ID NO: 1, such as at least 86% or at least 87%, 88%, 89%, 90%, 91%, 92%, 93%, 94%, 95%, 96%, 97%, 98% or at least 99% sequence identity. In yet another preferred embodiment, the targeting unit consists of the amino acid sequence 24-93 of SEQ ID NO: 1.

In some embodiments, the targeting unit comprises the amino acid sequence 24-93 of SEQ ID NO: 1, except that at the most six amino acids have been substituted, deleted or inserted, such as at the most five amino acids, such as at the most four amino acids, such as at the most three amino acids, such as at the most two amino acids or such as at the most one amino acid. An embodiment of such a targeting unit is one comprising the amino acid sequence 26-93 of SEQ ID NO: 1 or one comprising the amino acid sequence 28-93 of SEQ ID NO: 1.

In other embodiments, the targeting unit consists of the amino acid sequence 24-93 of SEQ ID NO: 1, except that at the most six amino acids have been substituted, deleted or inserted, such as at the most five amino acids, such as at the most four amino acids, such as at the most three amino acids, such as at the most two amino acids or such as at the most one amino acid. An embodiment of such a targeting unit is one comprising the amino acid sequence 26-93 of SEQ ID NO: 1 or one comprising the amino acid sequence 28-93 of SEQ ID NO: 1.

In other embodiments, the targeting unit comprises a nucleotide sequence having at least 80% sequence identity to the nucleotide sequence with SEQ ID NO: 122.

In other embodiments, the targeting unit comprises a nucleotide sequence having at least 85% sequence identity to the nucleotide sequence with SEQ ID NO: 122, such as at least 86% or at least 87%, 88%, 89%, 90%, 91%, 92%, 93%, 94%, 95%, 96%, 97%, 98% or at least 99% sequence identity. In other embodiments, the targeting unit has the nucleotide sequence of SEQ ID NO: 122.

In some embodiments, the targeting unit consists of a nucleotide sequence having at least 80% sequence identity to the nucleotide sequence with SEQ ID NO: 122.

In other embodiments, the targeting unit consists of a nucleotide sequence having at least 85% sequence identity to the nucleotide sequence 70-277 of SEQ ID NO: 122, such as at least 86% or at least 87%, 88%, 89%, 90%, 91%, 92%, 93%, 94%, 95%, 96%, 97%, 98% or at least 99% sequence identity. In yet another preferred embodiment, the targeting unit has the nucleotide sequence of SEQ ID NO: 122.

In other embodiments, the targeting unit comprises or is anti-pan HLA class II. This targeting unit induces rapid and strong antibody responses with mixed IgG1 and IgG2a antibodies. Moreover, it induces a significant cellular response, i.e. CD4+ T cell responses and CD8+ T cell responses.

In some embodiments, the targeting unit comprises an amino acid sequence having at least 80% sequence identity to the amino acid sequence 20-260 of SEQ ID NO: 2.

In some preferred embodiments, the targeting unit comprises an amino acid sequence having at least 85% sequence identity to the amino acid sequence 20-260 of SEQ ID NO: 1, such as at least 86% or at least 87%, 88%, 89%, 90%, 91%, 92%, 93%, 94%, 95%, 96%, 97%, 98% or at least 99% sequence identity. In yet a further preferred embodiment, the targeting unit has the amino acid sequence 20-260 of SEQ ID NO: 2.

In other embodiments, the targeting unit consists of an amino acid sequence having at least 80% sequence identity to the amino acid sequence 20-260 of SEQ ID NO: 2.

In some embodiments, the targeting unit consists of an amino acid sequence having at least 85% sequence identity to the amino acid sequence 20-260 of SEQ ID NO: 2, such as at least 86% or at least 87%, 88%, 89%, 90%, 91%, 92%, 93%, 94%, 95%, 96%, 97%, 98% or at least 99% sequence identity. In yet another preferred embodiment, the targeting unit consists of the amino acid sequence 20-260 of SEQ ID NO: 2.

In some embodiments, the targeting unit comprises the amino acid sequence 20-260 of SEQ ID NO: 2, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2 amino acids or such as at the most one amino acid.

In other embodiments, the targeting unit consists of the amino acid sequence 20-260 of SEQ ID NO: 2, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2 amino acids or such as at the most one amino acid.

In other embodiments, the targeting unit comprises or consists of a nucleotide sequence having at least 80% sequence identity to the nucleotide sequence with SEQ ID NO: 141.

In other embodiments, the targeting unit comprises or consists of a nucleotide sequence having at least 85% sequence identity to the nucleotide sequence with SEQ ID NO: 141, such as at least 86% or at least 87%, 88%, 89%, 90%, 91%, 92%, 93%, 94%, 95%, 96%, 97%, 98% or at least 99% sequence identity. In other embodiments, the targeting unit has the nucleotide sequence of SEQ ID NO: 141.

### Signal Peptide

In some embodiments, the construct of the disclosure is a polynucleotide which further comprises a nucleotide sequence encoding a signal peptide. The signal peptide is either located at the N-terminal end of the targeting unit or the C-terminal end of the targeting unit, depending on the orientation of the targeting unit in the polypeptide (FIG. 1). The signal peptide is designed to allow secretion of the polypeptide encoded by the nucleic acid comprised in the polynucleotide in the cells transfected with said polynucleotide.

Any suitable signal peptide may be used. Examples of suitable peptides are an Ig VH signal peptide, a human TPA signal peptide, such as SEQ ID NO: 3 and a human MIP1-α signal peptide (corresponding to amino acids 24-93 of SEQ ID NO: 1).

In some embodiments, the polynucleotide comprises a nucleotide sequence encoding a human MIP1-α signal peptide (corresponding to amino acids 1-23 of SEQ ID NO: 1) and preferably comprises a nucleotide sequence encoding a human MIP1-α targeting unit (corresponding to amino acids 24-93 of SEQ ID NO: 1).

In some preferred embodiments, the polynucleotide comprises a nucleotide sequence encoding a signal peptide that comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity to the amino acid sequence 1-23 of SEQ ID NO: 1.

In other embodiments, the polynucleotide comprises a nucleotide sequence encoding a signal peptide that comprises the amino acid sequence 1-23 of SEQ ID NO: 1, except that at the most four amino acids have been substituted, deleted or inserted, such as at the most three amino acids, such as at the most two amino acids or such as at the most one amino acid.

In other preferred embodiments, the polynucleotide comprises a nucleotide sequence encoding a signal peptide that comprises the amino acid sequence 1-23 of SEQ ID NO: 1.

In other preferred embodiments, the polynucleotide comprises a nucleotide sequence encoding a signal peptide that consists of an amino acid sequence having at least 80%, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% to the amino acid sequence 1-23 of SEQ ID NO: 1.

In other embodiments, the polynucleotide comprises a nucleotide sequence encoding a signal peptide with the amino acid sequence 1-23 of SEQ ID NO: 1.

In some embodiments, the polynucleotide comprises a nucleotide sequence encoding a signal peptide, wherein said nucleotide sequence has at least 80% sequence identity to the nucleotide sequence with SEQ ID NO: 123.

In further embodiments, the polynucleotide comprises a nucleotide sequence encoding a signal peptide, wherein said nucleotide sequence has at least 85% sequence identity to the nucleotide sequence with SEQ ID NO: 123, such as at least 86% or at least 87%, 88%, 89%, 90%, 91%, 92%, 93%, 94%, 95%, 96%, 97%, 98% or at least 99% sequence identity.

In yet further embodiments, the polynucleotide comprises a nucleotide sequence encoding a signal peptide, wherein said nucleotide sequence is SEQ ID NO: 123.

In other embodiments, the polynucleotide comprises a nucleotide sequence encoding an Ig VH signal peptide (SEQ ID NO: 132, corresponding to amino acids 1-19 of SEQ ID NO: 2) and preferably further comprises a nucleotide sequence encoding an anti-pan HLA class II targeting unit (corresponding to nucleotides 1 to 57 of SEQ ID NO: 141).

In further embodiments, the polynucleotide comprises a nucleotide sequence encoding a signal peptide that comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%, sequence identity to the amino acid sequence 1-19 of SEQ ID NO: 2 or to SEQ ID NO: 132.

In other embodiments, the polynucleotide comprises a nucleotide sequence encoding a signal peptide that comprises the amino acid sequence 1-19 of SEQ ID NO: 2, except that at the most four amino acids have been substituted, deleted or inserted, such as at the most three amino acids, such as at the most two amino acids or such as at the most one amino acid.

In other embodiments, the polynucleotide comprises a nucleotide sequence encoding a signal peptide that comprises the amino acid sequence 1-19 of SEQ ID NO: 2.

In some embodiments, the polynucleotide comprises a nucleotide sequence encoding a signal peptide that consists of an amino acid sequence having at least 80%, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% to the amino acid sequence 1-19 of SEQ ID NO: 2.

In other embodiments, the polynucleotide comprises a nucleotide sequence encoding a signal peptide with the amino acid sequence 1-19 of SEQ ID NO: 2.

In some embodiments, the polynucleotide comprises a nucleotide sequence encoding a signal peptide, wherein said nucleotide sequence has at least 80% sequence identity to the nucleotide sequence 1-57 of SEQ ID NO: 141.

In further embodiments, the polynucleotide comprises a nucleotide sequence encoding a signal peptide, wherein said nucleotide sequence has at least 85% sequence identity to the nucleotide sequence 1-57 of SEQ ID NO: 141, such as at least 86% or at least 87%, 88%, 89%, 90%, 91%, 92%, 93%, 94%, 95%, 96%, 97%, 98% or at least 99% sequence identity.

In yet further embodiments, the polynucleotide comprises a nucleotide sequence encoding a signal peptide, wherein said nucleotide sequence is the nucleotide sequence 1-57 of SEQ ID NO: 141.

### Sequence Identity

Sequence identity may be determined as follows: A high level of sequence identity indicates likelihood that a second sequence is derived from a first sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned. The number of fully conserved residues is counted and divided by the length of the reference polypeptide. In doing so, any tags or fusion protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalize to gaps in the end of the sequences. When utilizing the ALIGN and Align0 program for comparing amino acid sequences, a BLOSUM50 substitution matrix with gap opening/extension penalties of −12/−2 is preferably used.

Amino acid sequence variants may be prepared by introducing appropriate changes into the nucleotide sequence encoding the vaccine, or by peptide synthesis. Such modifications include, for example, deletions from, and/or insertions into and/or substitutions of, residues within the amino acid sequences. The terms substituted/substitution, deleted/deletions and inserted/insertions as used herein in reference to amino acid sequences and sequence identities are well known and clear to the skilled person in the art. Any combination of deletion, insertion, and substitution can be made to arrive at the final construct, provided that the final construct possesses the desired characteristics. For example, deletions, insertions or substitutions of amino acid residues may produce a silent change and result in a functionally equivalent peptide/polypeptide.

Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Herein encompassed are conservative substitutions, i.e. like-for-like substitution such as basic for basic, acidic for acidic, polar for polar etc. and non-conservative substitutions, i.e. from one class of residue to another or alternatively involving the inclusion of unnatural amino acids such as ornithine, diaminobutyric acid ornithine, norleucine, ornithine, pyriylalanine, thienylalanine, naphthylalanine and phenylglycine. Conservative substitutions that may be made are, for example within the groups of basic amino acids (arginine, lysine and histidine), acidic amino acids (glutamic acid and aspartic acid), aliphatic amino acids (alanine, aaline, leucine, isoleucine), polar amino acids (glutamine, asparagine, serine, threonine), aromatic amino acids (phenylalanine, tryptophan, tyrosine), hydroxyl amino acids (serine, threonine), large amino acids (phenylalanine, tryptophan) and small amino acids (glycine, alanine).

Substitutions may also be made by unnatural amino acids and substituting residues include; alpha* and alpha-disubstituted* amino acids, N-alkyl amino acids*, lactic acid*, halide derivatives of natural amino acids such as trifluorotyrosine*, p-CI-phenylalanine*, p-Br-phenylalanine*, p-I-phenylalanine*, L-allyl-glycine*, β-alanine*, L-a-amino butyric acid*, L-y-amino butyric acid*, L-a-amino isobutyric acid*, L-e-amino caproic acid*, 7-amino heptanoic acid*, L-methionine sulfone*, L-norleucine*, L-norvaline*, p-nitro-L-phenylalanine*, L-hydroxyproline*, L-thioproline*, methyl derivatives of phenylalanine (Phe) such as 4-methyl-Phe*, pentamethyl-Phe*, L-Phe (4-amino) #, L-Tyr (methyl)*, L-Phe (4-isopropyl)*, L-Tic (1,2,3,4-tetrahydroisoquinoline-3-carboxyl acid)*, L-diaminopropionic acid * and L-Phe (4-benzyl)*.

In the paragraph above,* indicates the hydrophobic nature of the substituting residue, whereas #indicates the hydrophilic nature of substituting residue and #* indicates amphipathic characteristics of the substituting residue. Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation involves the presence of one or more amino acid residues in peptoid form.

### Polynucleotides

The construct of the disclosure may be in the form of a polynucleotide, e.g. DNA or RNA, including genomic DNA, cDNA and mRNA, either double-stranded or single-stranded. In preferred embodiments, the construct is a DNA construct, i.e. the polynucleotide is a DNA.

A further aspect of the disclosure is a polynucleotide comprising a nucleotide sequence encoding a polypeptide comprising:
a) a targeting unit targeting or capable of targeting antigen-presenting cells,
b) a multimerization unit such as a dimerization unit, and
c) an antigenic unit comprising one or more T cell epitopes and one or more antigens or parts or fragments thereof,
   wherein the antigenic unit comprises a subunit comprising the T cell epitopes which are separated from each other by T cell linkers, if more than one T cell epitope is comprised in the subunit, and wherein the subunit is connected to the multimerization unit (or dimerization unit) by a unit linker and separated from the one or more antigens or parts or fragments thereof by an antigen linker. Preferably, the one or more T cell epitopes and the one or more antigens or parts or fragments thereof are from a pathogen.

The polynucleotide may be a DNA or an RNA polynucleotide, including genomic DNA, cDNA and mRNA, either double-stranded or single-stranded. In a preferred embodiment, the construct is a DNA plasmid, i.e. the polynucleotide is a DNA.

In some embodiments the polynucleotide is optimized for use in the species to which it is administered. For administration to a human, in some embodiments the polynucleotide sequence is human codon optimized.

### Vectors

The polynucleotide sequence of the construct may be a DNA polynucleotide comprised in a vector suitable for transfecting a host cell and expression of a polypeptide or multimeric/dimeric protein encoded by the polynucleotide, i.e. an expression vector, such as a DNA plasmid. In other preferred embodiments, the vector is a viral vector, such as a retroviral vector. In other embodiments, the vector is suitable for transfecting a host cell and expression of an mRNA encoding for the polypeptide/multimeric protein.

A further aspect of the disclosure is a vector comprising a polynucleotide comprising a nucleotide sequence encoding a polypeptide comprising:
a) a targeting unit targeting or capable of targeting antigen-presenting cells;
b) a multimerization unit such as a dimerization unit; and
c) an antigenic unit comprising one or more T cell epitopes and one or more antigens or parts or fragments thereof;
   wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker. Preferably, the one or more T cell epitopes and the one or more antigens or parts or fragments thereof are from a pathogen, as detailed herein above.

Thus, herein is disclosed a vector comprising a polynucleotide comprising a nucleotide sequence encoding a polypeptide comprising:
a) a targeting unit targeting or capable of targeting antigen-presenting cells;
b) a multimerization unit; and
c) an antigenic unit comprising one or more T cell epitopes and one or more antigens or parts or fragments thereof;
   wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker. Preferably, the one or more T cell epitopes and the one or more antigens or parts or fragments thereof are from a pathogen, as detailed herein above.

Also disclosed is a vector comprising a polynucleotide comprising a nucleotide sequence encoding a polypeptide comprising:
a) a targeting unit targeting or capable of targeting antigen-presenting cells;
b) a dimerization unit; and
c) an antigenic unit comprising one or more T cell epitopes and one or more antigens or parts or fragments thereof;
   wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker. Preferably, the one or more T cell epitopes and the one or more antigens or parts or fragments thereof are from a pathogen, as detailed herein above.

Preferably, the vector allows for easy exchange of the various units described above, particularly the antigenic unit.

In some embodiments, the vector is a pUMVC4a vector or a vector comprising NTC9385R vector backbones. The antigenic unit may be exchanged with an antigenic unit cassette restricted by the SfiI restriction enzyme cassette where the 5′ site is incorporated in the nucleotide sequence encoding the GLGGL (SEQ ID NO: 42)/GLSGL (SEQ ID NO: 85) unit linker and the 3′ site is included after the stop codon in the vector.

In preferred embodiments, the vector is a DNA plasmid and the polynucleotide is DNA.

### Host Cell

A further aspect of the disclosure is a host cell comprising:
(i) a polynucleotide comprising: a nucleotide sequence encoding a polypeptide a targeting unit targeting or capable of targeting antigen-presenting cells; a multimerization unit such as a dimerization unit; and an antigenic unit comprising one or more T cell epitopes and one or more antigens or parts or fragments thereof, wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker; or
(ii) a polypeptide comprising the nucleotide sequence defined in (i).

Preferably, the one or more T cell epitopes and the one or more antigens or parts or fragments thereof are from a pathogen, as detailed herein above.

Also disclosed herein is a host cell comprising:
(i) a polynucleotide comprising: a nucleotide sequence encoding a polypeptide a targeting unit targeting or capable of targeting antigen-presenting cells; a multimerization unit; and an antigenic unit comprising one or more T cell epitopes and one or more antigens or parts or fragments thereof, wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker; or
(ii) a polypeptide comprising the nucleotide sequence defined in (i).

Preferably, the one or more T cell epitopes and the one or more antigens or parts or fragments thereof are from a pathogen, as detailed herein above.

Also disclosed herein is a host cell comprising:
(i) a polynucleotide comprising: a nucleotide sequence encoding a polypeptide a targeting unit targeting or capable of targeting antigen-presenting cells; a dimerization unit; and an antigenic unit comprising one or more T cell epitopes and one or more antigens or parts or fragments thereof, wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker; or
(ii) a polypeptide comprising the nucleotide sequence defined in (i).

Preferably, the one or more T cell epitopes and the one or more antigens or parts or fragments thereof are from a pathogen, as detailed herein above.

Suitable host cells include prokaryotes, yeast, insect or higher eukaryotic cells. In a preferred embodiment, the host cell is a human cell, preferably a cell of a human individual in need of the vaccine of the disclosure.

### Pharmaceutical Compositions

Further embodiments of the present disclosure provide a pharmaceutical composition comprising a construct, polynucleotide, polypeptide, multimeric protein or dimeric protein as disclosed herein, and one or more pharmaceutically acceptable carriers. In some embodiments the pharmaceutical composition further comprises one or more pharmaceutically acceptable excipients, and/or diluents.

Suitable pharmaceutically acceptable carriers include, but are not limited to, saline, buffered saline, such as PBS, dextrose, water, glycerol, ethanol, sterile isotonic aqueous buffers, and combinations thereof.

The pharmaceutical composition may further comprise an adjuvant. Particularly for pharmaceutical compositions comprising the multimeric protein or dimeric protein, pharmaceutically acceptable adjuvants include, but are not limited to poly-ICLC, 1018 ISS, aluminum salts, Amplivax, AS 15, BCG, CP-870,893, CpG7909, CyaA, dSLIM, GM-CSF, IC30, IC31, Imiquimod, ImuFact EV1 P321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PLGA microparticles, resiquimod, SRL172, virosomes and other virus-like particles, YF-17D, VEGF trap, R848, beta-glucan, Pam3Cys, Aquila's QS21 stimulon, vadimezan, and/or AsA404 (DMXAA). However, as detailed above, due to the presence of the targeting units, the composition can be administered without additional adjuvant; thus, in some embodiments, the composition does not comprise an adjuvant.

In some specific embodiments the composition may comprise a pharmaceutically acceptable amphiphilic block co-polymer comprising blocks of poly(ethylene oxide) and polypropylene oxide).

An “amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (poloxamers), PPO PEO PPO, (PEO PPO-)4ED (a poloxamine), and (PPO PEO-)4ED (a reverse poloxamine), where “ED” is an ethylenediaminyl group.

A “poloxamer” is a linear amphiphilic block co-polymer constituted by one block of poly(ethylene oxide) coupled to one block of poly(propylene oxide) coupled to one block of PEO, i.e. a structure of the formula EOa-POb-EOa, where EO is ethylene oxide, PO is propylene oxide, a is an integer from 2 to 130, and b is an integer from 15 to 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of a molecular weight of about 1800 (corresponding to b being about 31 PPO) and approximately 80% (w/w) of PEO (corresponding to a being about 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) is an X-shaped block co-polymers that bears four PEO-PPO arms connected to a central ethylenediamine moiety via bonds between the free OH groups comprised in the PEO-PPO-arms and the primary amine groups in ethylenediamine moiety. Reverse poloxamines are likewise X-shaped block co-polymers that bear four PPO-PEO arms connected to a central ethylenediamine moiety via bonds between the free OH groups comprised in the PPO-PEO arms and the primary amine groups in ethylenediamine.

Preferred amphiphilic block co-polymers are poloxamers or poloxamines. Preferred are poloxamer 407 and 188, in particular poloxamer 188. Preferred poloxamines are sequential poloxamines of formula (PEO-PPO)4-ED. Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

In some embodiments, the composition comprises the amphiphilic block co-polymer in an amount of from 0.2% w/v to 20% w/v, such as of from 0.2% w/v to 18% w/v, 0.2% w/v to 16% w/v, 0.2% w/v to 14% w/v, 0.2% w/v to 12% w/v, 0.2% w/v to 10% w/v, 0.2% w/v to 8% w/v, 0.2% w/v to 6% w/v, 0.2% w/v to 4% w/v, 0.4% w/v to 18% w/v, 0.6% w/v to 18% w/v, 0.8% w/v to 18% w/v, 1% w/v to 18% w/v, 2% w/v to 18% w/v, 1% w/v to 5% w/v, or 2% w/v to 4% w/v. Particularly preferred are amounts in the range of from 0.5% w/v to 5% w/v. In other embodiments, the composition comprises the amphiphilic block co-polymer in an amount of from 2% w/v to 5% w/v, such as about 3% w/v.

For pharmaceutical compositions comprising the polynucleotide or vector, the pharmaceutical compositions may comprise molecules that ease transfection of cells.

The pharmaceutical composition may be formulated in any way suitable for administration to a subject, e.g. such as a liquid formulation for injection, e.g. for intradermal or intramuscular injection.

The pharmaceutical composition may be administered in any way suitable for administration to a subject, such as administered by intradermal, intramuscular, or subcutaneous injection, or by mucosal or epithelial application, such as intranasal or oral.

In preferred embodiments, the pharmaceutical composition comprises a polynucleotide, e.g. comprised in a vector such as apolycistronic vector, and is administered by intramuscular or intradermal injection.

The pharmaceutical composition of the disclosure typically comprises the polynucleotide in a range of from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg. The pharmaceutical composition of the disclosure typically comprises the polypeptide, dimeric protein and/or multimeric protein in the range of from 5 μg to 5 mg.

The amount of polynucleotide, polypeptide, multimeric protein or dimeric protein may vary depending on whether the pharmaceutical composition is administered for prophylactic or therapeutic treatment, the severity of the disease in individuals which are infected, and on parameters like the age, weight, gender, medical history and pre-existing conditions.

### Vaccines

The construct of the disclosure may be administered to a subject as a vaccine, i.e. a pharmaceutical composition comprising the construct, e.g. in the form of a polynucleotide, vector, polypeptide, multimeric protein or dimeric protein and a pharmaceutically acceptable carrier.

A further aspect of the disclosure is a vaccine, comprising a pharmaceutically acceptable carrier and
(i) a polynucleotide comprising a nucleotide sequence encoding a polypeptide comprising: a targeting unit targeting or capable of targeting antigen-presenting cells; a multimerization unit such as a dimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of two polypeptides as defined in (ii), such as a dimeric protein consisting of two polypeptides as defined in (ii);
wherein the antigenic unit comprises a subunit comprising the T cell epitopes which are separated from each other by T cell epitope linkers, if more than one T cell epitope is comprised in the subunit; and wherein the subunit is connected to the multimerization unit by a unit linker and separated from the one or more antigens or parts or fragments thereof by an antigen linker. Preferably, the one or more T cell epitopes and the one or more antigens or parts or fragments thereof are from a pathogen, as detailed herein above.

Also disclosed herein is a vaccine, comprising a pharmaceutically acceptable carrier and
(i) a polynucleotide comprising a nucleotide sequence encoding a polypeptide comprising: a targeting unit targeting or capable of targeting antigen-presenting cells; a multimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii);
wherein the antigenic unit comprises a subunit comprising the T cell epitopes which are separated from each other by T cell epitope linkers, if more than one T cell epitope is comprised in the subunit; and wherein the subunit is connected to the multimerization unit by a unit linker and separated from the one or more antigens or parts or fragments thereof by an antigen linker. Preferably, the one or more T cell epitopes and the one or more antigens or parts or fragments thereof are from a pathogen, as detailed herein above.

Also disclosed herein is a vaccine, comprising a pharmaceutically acceptable carrier and
(i) a polynucleotide comprising a nucleotide sequence encoding a polypeptide comprising: a targeting unit targeting or capable of targeting antigen-presenting cells; a dimerization unit; and an antigenic unit comprising one or more T cell epitopes and one or more antigens or parts or fragments thereof; or
(ii) a polypeptide encoded by the nucleotide sequence defined in (i); or
(iii) a dimeric protein consisting of two polypeptides as defined in (ii);
wherein the antigenic unit comprises a subunit comprising the T cell epitopes which are separated from each other by T cell epitope linkers, if more than one T cell epitope is comprised in the subunit; and wherein the subunit is connected to the dimerization unit by a unit linker and separated from the one or more antigens or parts or fragments thereof by an antigen linker. Preferably, the one or more T cell epitopes and the one or more antigens or parts or fragments thereof are from a pathogen, as detailed herein above.

The targeting unit, dimerization unit, antigenic unit, T cell epitopes, T cell epitope linkers, unit linker and antigen linker are as described herein elsewhere.

Suitable pharmaceutically acceptable carriers include, but are not limited to, saline, buffered saline, such as PBS, dextrose, water, glycerol, ethanol, sterile isotonic aqueous buffers, and combinations thereof.

In some embodiments, the pharmaceutically acceptable carrier or diluent is an aqueous buffer. In other embodiments, the aqueous buffer is Tyrode's buffer, e.g. Tyrode's buffer comprising 140 mM NaCl, 6 mM KCl, 3 mM CaCl2), 2 mM MgCl2, 10 mM 4-(2-hydroxyethyl)-1-piperazineethanesulfonic acid (Hepes) pH 7.4, and 10 mM glucose.

The vaccine may further comprise an adjuvant. Particularly for vaccines comprising multimeric proteins, such as dimeric proteins, pharmaceutically acceptable adjuvants include, but are not limited to poly-ICLC, 1018 ISS, aluminum salts, Amplivax, AS 15, BCG, CP-870,893, CpG7909, CyaA, dSLIM, GM-CSF, IC30, IC31, Imiquimod, ImuFact EV1 P321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PLGA microparticles, resiquimod, SRL172, virosomes and other virus-like particles, YF-17D, VEGF trap, R848, beta-glucan, Pam3Cys, Aquila's QS21 stimulon, vadimezan, and/or AsA404 (DMXAA). However, as detailed above, due to the presence of the targeting unit, the present vaccines can be administered without additional adjuvant; thus, in some embodiments, the vaccine does not comprise an adjuvant, i.e. is adjuvant-free.

In some specific embodiments the vaccine may comprise a pharmaceutically acceptable amphiphilic block co-polymer comprising blocks of poly(ethylene oxide) and polypropylene oxide).

An “amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (poloxamers), PPO PEO PPO, (PEO PPO-)4ED (a poloxamine), and (PPO PEO-)4ED (a reverse poloxamine), where “ED” is an ethylenediaminyl group.

A “poloxamer” is a linear amphiphilic block co-polymer constituted by one block of poly(ethylene oxide) coupled to one block of poly(propylene oxide) coupled to one block of PEO, i.e. a structure of the formula EOa-POb-EOa, where EO is ethylene oxide, PO is propylene oxide, a is an integer from 2 to 130, and b is an integer from 15 to 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of a molecular weight of about 1800 (corresponding to b being about 31 PPO) and approximately 80% (w/w) of PEO (corresponding to a being about 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) is an X-shaped block co-polymers that bears four PEO-PPO arms connected to a central ethylenediamine moiety via bonds between the free OH groups comprised in the PEO-PPO-arms and the primary amine groups in ethylenediamine moiety. Reverse poloxamines are likewise X-shaped block co-polymers that bear four PPO-PEO arms connected to a central ethylenediamine moiety via bonds between the free OH groups comprised in the PPO-PEO arms and the primary amine groups in ethylenediamine.

Useful amphiphilic block co-polymers are poloxamers or poloxamines, more particularly poloxamer 407 and 188, and in particular poloxamer 188. Useful poloxamines are sequential poloxamines of formula (PEO-PPO)4-ED; in particular, preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

In some embodiments, the vaccine comprises the amphiphilic block co-polymer in an amount of from 0.2% w/v to 20% w/v, such as of from 0.2% w/v to 18% w/v, 0.2% w/v to 16% w/v, 0.2% w/v to 14% w/v, 0.2% w/v to 12% w/v, 0.2% w/v to 10% w/v, 0.2% w/v to 8% w/v, 0.2% w/v to 6% w/v, 0.2% w/v to 4% w/v, 0.4% w/v to 18% w/v, 0.6% w/v to 18% w/v, 0.8% w/v to 18% w/v, 1% w/v to 18% w/v, 2% w/v to 18% w/v, 1% w/v to 5% w/v, or 2% w/v to 4% w/v. Particularly preferred are amounts in the range of from 0.5% w/v to 5% w/v. In other embodiments, the vaccine comprises the amphiphilic block co-polymer in an amount of from 2% w/v to 5% w/v, such as about 3% w/v.

For vaccines comprising polynucleotides, the vaccines may further comprise molecules that ease transfection of cells.

The vaccine may be formulated in any way suitable for administration to a subject, e.g. a patient suffering or suspected of suffering from an infection caused by a pathogen, such as a liquid formulation for injection, e.g. for intradermal or intramuscular injection.

The vaccine, comprising in some embodiments a polynucleotide as described herein, may be administered in any way suitable for administration to a subject, such as administered by intradermal, intramuscular, or subcutaneous injection, or by mucosal or epithelial application, such as intranasal or oral administration.

In some embodiments, the vaccine comprises a polynucleotide as described herein and is administered by intramuscular or intradermal injection.

The vaccine of the disclosure typically comprises the polynucleotide in a range of from 0.1 μg to 10 mg, e.g. about 0.2 μg, 0.3 μg, 0.4 μg, 0.5 μg, 0.75 μg, 1 μg, 5 μg, 10 μg, 25 μg, 50 μg, 75 μg, or more; such as from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg. The vaccine of the disclosure typically comprises the polypeptide or multimeric/dimeric protein in the range of from 5 μg to 5 mg.

The amount of polynucleotide/polypeptide/multimeric protein/dimeric protein may vary depending on whether the vaccine is administered for prophylactic or therapeutic treatment, the severity of the disease in individuals which are infected, and on parameters like the age, weight, gender, medical history and pre-existing conditions.

### Polypeptides and Multimeric/Dimeric Proteins

The construct of the disclosure may be in the form of a polypeptide encoded by the nucleotide sequence comprised in the polynucleotide as described above.

A further aspect of the disclosure is a polypeptide, comprising:
a) a targeting unit targeting or capable of targeting antigen-presenting cells,
b) a multimerization unit such as a dimerization unit, and
c) an antigenic unit comprising one or more T cell epitopes and one or more antigens or parts or fragments thereof,
   wherein the antigenic unit comprises a subunit comprising the T cell epitopes which are separated from each other by T cell linkers, if more than one T cell epitope is comprised in the subunit, and wherein the subunit is connected to the multimerization/dimerization unit by a unit linker and separated from the one or more antigens or parts or fragments thereof by an antigen linker. Preferably, the one or more T cell epitopes and the one or more antigens or parts or fragments thereof are from a pathogen.

The polypeptide may be expressed in vitro for production of a vaccine, or the polypeptide may be expressed in vivo as a result of the administration of the polynucleotide to a subject, as described above.

Due to the presence of the multimerization/dimerization unit, multimeric/dimeric proteins are formed when the polypeptide is expressed, i.e. by joining multiple polypeptides (such as two polypeptides in embodiments with a dimerization unit) via their respective multimerization units.

A further aspect of the disclosure is a multimeric protein comprising multiple polypeptides, such as a dimeric protein comprising two polypeptides, each comprising:
a) a targeting unit for targeting antigen-presenting cells;
b) a multimerization unit such as a dimerization unit; and
c) an antigenic unit comprising one or more T cell epitopes and one or more antigens or parts or fragments thereof;
   wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker. Preferably, the one or more T cell epitopes and the one or more antigens or parts or fragments thereof are from a pathogen.

Herein is thus disclosed a multimeric protein comprising multiple polypeptides, each comprising:
a) a targeting unit for targeting antigen-presenting cells;
b) a multimerization unit; and an antigenic unit comprising one or more T cell epitopes, and
c) one or more antigens or parts or fragments thereof;
   wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker. Preferably, the one or more T cell epitopes and the one or more antigens or parts or fragments thereof are from a pathogen.

The multimeric protein may be a homomultimer, i.e. a multimeric protein wherein the multiple polypeptide chains are identical and consequently comprise identical units and thus identical antigen sequences, or the multimeric protein may be a heteromultimer comprising multiple polypeptide chains, wherein each polypeptide chain may comprise different antigen sequences in its antigenic unit. The latter may be relevant if the number of antigens for inclusion into the antigenic unit would exceed an upper size limit for the antigenic unit. It is preferred that the multimeric protein is a homomultimeric protein.

Herein is also disclosed a dimeric protein comprising two polypeptides, each comprising:
a) a targeting unit for targeting antigen-presenting cells;
b) a dimerization unit; and
c) an antigenic unit comprising one or more T cell epitopes and one or more antigens or parts or fragments thereof;
   wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker. Preferably, the one or more T cell epitopes and the one or more antigens or parts or fragments thereof are from a pathogen.

The dimeric protein may be a homodimer, i.e. a dimeric protein wherein the two polypeptide chains are identical and consequently comprise identical units and thus identical antigen sequences, or the dimeric protein may be a heterodimer comprising two polypeptide chains, wherein polypeptide chain 1 comprises different antigen sequences in its antigenic unit than polypeptide 2. The latter may be relevant if the number of antigens for inclusion into the antigenic unit would exceed an upper size limit for the antigenic unit. It is preferred that the dimeric protein is a homodimeric protein.

### Methods for Preparing the Vaccine

Suitable methods for preparing the vaccine according to the disclosure are disclosed in WO 2004/076489A1, WO 2011/161244A1, WO 2013/092875A1, WO 2017/118695A1 and WO 2021/205027 A1, which are incorporated herein by reference, in particular page 15, lines 10-13 and page 17, section “Construction of Vaccibodies” of WO 2004/076489A1; page 10, lines 10-14 and Example 1 of WO 2011/161244A1; page 15 and Example 1 of WO 2013/092875A1; section “Methods for preparing the vaccine” and Example 1 of WO 2017/118695A1 and page 26, line 17 to page 27, line 38, page 30, line 23 to page 31, line 27 and Example 4 of WO 2021/205027 A1.

In one aspect, the disclosure relates to a method for preparing a vaccine comprising the multimeric protein, such as the dimeric protein, or the polypeptide as defined above by producing the polypeptides in vitro. The in vitro synthesis of the polypeptides and proteins may be carried out by any suitable method known to the person skilled in the art, such as by peptide synthesis or expression of the polypeptide in a variety of expressions systems followed by purification.

Thus, a further aspect of the disclosure is a method for preparing a vaccine which comprises a multimeric protein consisting of multiple polypeptides, such as a dimeric protein consisting of two polypeptides; or which comprises a polypeptide,
wherein the method comprises:
   a) transfecting cells with a polynucleotide comprising: a targeting unit targeting or capable of targeting antigen-presenting cells; a multimerization unit such as a dimerization unit; and an antigenic unit comprising one or more T cell epitopes and one or more antigens or parts or fragments thereof; wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker;
   b) culturing the cells;
   c) collecting and purifying the multimeric protein or the polypeptide expressed from the cells; and
   d) mixing the multimeric protein or polypeptide obtained from step c) with a pharmaceutically acceptable carrier.

Preferably, the one or more T cell epitopes and the one or more antigens or parts or fragments thereof are from a pathogen. In some embodiments, the multimeric protein, such as the dimeric protein, or polypeptide obtained from step c) is dissolved in said pharmaceutically acceptable carrier.

Also disclosed herein is a method for preparing a vaccine which comprises a multimeric protein consisting of multiple polypeptides; or which comprises a polypeptide,
wherein the method comprises:
   a) transfecting cells with a polynucleotide comprising: a targeting unit targeting or capable of targeting antigen-presenting cells; a multimerization unit; and an antigenic unit comprising one or more T cell epitopes and one or more antigens or parts or fragments thereof; wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker;
   b) culturing the cells;
   c) collecting and purifying the multimeric protein or the polypeptide expressed from the cells; and
   d) mixing the multimeric protein or polypeptide obtained from step c) with a pharmaceutically acceptable carrier.

Preferably, the one or more T cell epitopes and the one or more antigens or parts or fragments thereof are from a pathogen. In some embodiments, the multimeric protein, or polypeptide obtained from step c) is dissolved in said pharmaceutically acceptable carrier.

Also disclosed herein is a method for preparing a vaccine which comprises a dimeric protein consisting of two polypeptides; or which comprises a polypeptide,
wherein the method comprises:
   a) transfecting cells with a polynucleotide comprising: a targeting unit targeting or capable of targeting antigen-presenting cells; a dimerization unit; and an antigenic unit comprising one or more T cell epitopes and one or more antigens or parts or fragments thereof; wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker;
   b) culturing the cells;
   c) collecting and purifying the dimeric protein or the polypeptide expressed from the cells; and
   d) mixing the dimeric protein or polypeptide obtained from step c) with a pharmaceutically acceptable carrier.

Preferably, the one or more T cell epitopes and the one or more antigens or parts or fragments thereof are from a pathogen. In some embodiments, the dimeric protein, or polypeptide obtained from step c) is dissolved in said pharmaceutically acceptable carrier.

Purification may be carried out according to any suitable method, such as chromatography, centrifugation, or differential solubility.

In another aspect the disclosure relates to a method for preparing a vaccine comprising a polynucleotide comprising a nucleotide sequence encoding a polypeptide comprising: a targeting unit targeting or capable of targeting antigen-presenting cells; a multimerization unit such as a dimerization unit; and an antigenic unit comprising one or more T cell epitopes and one or more antigens or parts or fragments thereof, wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker, wherein the method comprises:
a) preparing the polynucleotide;
b) optionally cloning the polynucleotide into an expression vector; and
c) mixing the polynucleotide obtained from step a) or the vector obtained from step b) with a pharmaceutically acceptable carrier.

Preferably, the one or more T cell epitopes and the one or more antigens or parts or fragments thereof are from a pathogen.

Thus, also disclosed herein is a method for preparing a vaccine comprising a polynucleotide comprising a nucleotide sequence encoding a polypeptide comprising: a targeting unit targeting or capable of targeting antigen-presenting cells; a multimerization unit; and an antigenic unit comprising one or more T cell epitopes and one or more antigens or parts or fragments thereof; wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker, wherein the method comprises:
a) preparing the polynucleotide;
b) optionally cloning the polynucleotide into an expression vector; and
c) mixing the polynucleotide obtained from step a) or the vector obtained from step b) with a pharmaceutically acceptable carrier.

Preferably, the one or more T cell epitopes and the one or more antigens or parts or fragments thereof are from a pathogen.

Also disclosed herein is a method for preparing a vaccine comprising a polynucleotide comprising a nucleotide sequence encoding a polypeptide comprising: a targeting unit targeting or capable of targeting antigen-presenting cells; a dimerization unit; and an antigenic unit comprising one or more T cell epitopes and one or more antigens or parts or fragments thereof; wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker,
wherein the method comprises:
   a) preparing the polynucleotide;
   b) optionally cloning the polynucleotide into an expression vector; and
   c) mixing the polynucleotide obtained from step a) or the vector obtained from step b) with a pharmaceutically acceptable carrier.

Preferably, the one or more T cell epitopes and the one or more antigens or parts or fragments thereof are from a pathogen.

The polynucleotide may be prepared by any suitable method known to the skilled person. For example, the polynucleotide may be prepared by chemical synthesis using an oligonucleotide synthesizer.

In particular, shorter nucleotide sequences, e.g. nucleotide sequences encoding the targeting unit, the multimerization/dimerization unit and/or the subunit of the antigenic unit may be synthesized individually and then ligated into a vector backbone to produce the final polynucleotide.

In one aspect, the disclosure relates to the use of the construct, the polynucleotide, the polypeptide or the multimeric protein, such as a dimeric protein, described above as a medicament. Also disclosed herein is the use of the construct, the polynucleotide, the polypeptide or the multimeric protein described above as a medicament. Also disclosed herein is the use of the construct, the polynucleotide, the polypeptide or the dimeric protein described above as a medicament.

### Treatment

The construct and vaccine of the disclosure may be used to treat any infectious disease caused by any pathogen, including diseases caused by viruses (such as betacoronaviruses or influenza viruses or HIV), bacteria, fungi or parasites, and treatment may either be for prophylactic or for therapeutic purpose.

The construct/vaccine is administered such that it induces an immunoprotective response (for a prophylactic treatment) or an immunotherapeutic response (for a therapeutic treatment) in the individual vaccinated with such vaccine. Such response is induced by either a single vaccination or several vaccinations, e.g. an initial vaccination and one or several booster vaccinations, adequately spaced in time.

In a further aspect, the disclosure provides a method for treating a subject having an infectious disease or being in need of prevention of an infectious disease, the method comprising administering to the subject a vaccine comprising
(i) a polynucleotide comprising a nucleotide sequence encoding a polypeptide comprising: a targeting unit targeting or capable of targeting antigen-presenting cells; a multimerization unit, such as a dimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
wherein the antigenic unit comprises a subunit comprising the T cell epitopes which are separated from each other by T cell linkers, if more than one T cell epitope is comprised in the subunit;
and wherein the subunit is connected to the multimerization unit by a unit linker and separated from the one or more antigens or parts or fragments thereof by an antigen linker.

Preferably, the one or more T cell epitopes and the one or more antigens or parts or fragments thereof are from a pathogen.

Also disclosed herein is a method for treating a subject having an infectious disease or being in need of prevention of an infectious disease, the method comprising administering to the subject a vaccine comprising
(i) a polynucleotide comprising a nucleotide sequence encoding a polypeptide comprising: a targeting unit targeting or capable of targeting antigen-presenting cells; a multimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii);
wherein the antigenic unit comprises a subunit comprising the T cell epitopes which are separated from each other by T cell linkers, if more than one T cell epitope is comprised in the subunit;
and wherein the subunit is connected to the multimerization unit by a unit linker and separated from the one or more antigens or parts or fragments thereof by an antigen linker.

Preferably, the one or more T cell epitopes and the one or more antigens or parts or fragments thereof are from a pathogen.

Also disclosed herein is a method for treating a subject having an infectious disease or being in need of prevention of an infectious disease, the method comprising administering to the subject a vaccine comprising
(i) a polynucleotide comprising a nucleotide sequence encoding a polypeptide comprising: a targeting unit targeting or capable of targeting antigen-presenting cells; a dimerization unit; and an antigenic unit comprising one or more T cell epitopes and one or more antigens or parts or fragments thereof; or
(ii) a polypeptide encoded by the nucleotide sequence defined in (i); or
(iii) a dimeric protein consisting of two polypeptides as defined in (ii);
wherein the antigenic unit comprises a subunit comprising the T cell epitopes which are separated from each other by T cell linkers, if more than one T cell epitope is comprised in the subunit;
and wherein the subunit is connected to the dimerization unit by a unit linker and separated from the one or more antigens or parts or fragments thereof by an antigen linker.

Preferably, the one or more T cell epitopes and the one or more antigens or parts or fragments thereof are from a pathogen.

The vaccine, the targeting unit, the multimerization/dimerization unit, the antigenic unit, the subunit comprising T cell epitopes, and the linkers are described in detail herein above.

In yet a further aspect, the disclosure provides a vaccine for use in the prophylactic or therapeutic treatment of an infectious disease in a subject in need thereof, the vaccine comprising:
(i) a polynucleotide comprising a nucleotide sequence encoding a polypeptide comprising: a targeting unit targeting or capable of targeting antigen-presenting cells; a multimerization unit such as a dimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker,
wherein the vaccine is administered to said subject.

Preferably, the one or more T cell epitopes and the one or more antigens or parts or fragments thereof are from a pathogen.

Also disclosed herein is a vaccine for use in the prophylactic or therapeutic treatment of an infectious disease in a subject in need thereof, the vaccine comprising:
(i) a polynucleotide comprising a nucleotide sequence encoding a polypeptide comprising: a targeting unit targeting or capable of targeting antigen-presenting cells; a multimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii);
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker,
wherein the vaccine is administered to said subject.

Preferably, the one or more T cell epitopes and the one or more antigens or parts or fragments thereof are from a pathogen.

Also disclosed herein is a vaccine for use in the prophylactic or therapeutic treatment of an infectious disease in a subject in need thereof, the vaccine comprising:
(i) a polynucleotide comprising a nucleotide sequence encoding a polypeptide comprising: a targeting unit targeting or capable of targeting antigen-presenting cells; a dimerization unit; and an antigenic unit comprising one or more T cell epitopes and one or more antigens or parts or fragments thereof; or
(ii) a polypeptide encoded by the nucleotide sequence defined in (i); or
(iii) a dimeric protein consisting of two polypeptides as defined in (ii);
wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker,
wherein the vaccine is administered to said subject.

Preferably, the one or more T cell epitopes and the one or more antigens or parts or fragments thereof are from a pathogen.

The vaccine, the targeting unit, the multimerization/dimerization unit, the antigenic unit, the subunit comprising T cell epitopes, and the linkers are described in detail herein above.

Also disclosed herein is the use of a vaccine comprising:
(i) a polynucleotide comprising a nucleotide sequence encoding a polypeptide comprising: a targeting unit targeting or capable of targeting antigen-presenting cells; a multimerization unit such as a dimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker,
for the manufacture of a medicament for the treatment of a subject suffering from an infectious disease or being in need of prevention thereof, wherein the medicament is administered to said subject.

Also disclosed herein is a vaccine comprising:
(i) a polynucleotide comprising a nucleotide sequence encoding a polypeptide comprising: a targeting unit targeting or capable of targeting antigen-presenting cells; a multimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker,
for the manufacture of a medicament for the treatment of a subject suffering from an infectious disease or being in need of prevention thereof, wherein the medicament is administered to said subject.

Also disclosed herein is a vaccine comprising:
(i) a polynucleotide comprising a nucleotide sequence encoding a polypeptide comprising: a targeting unit targeting or capable of targeting antigen-presenting cells; a dimerization unit; and an antigenic unit comprising one or more T cell epitopes and one or more antigens or parts or fragments thereof; or
(ii) a polypeptide encoded by the nucleotide sequence defined in (i); or
(iii) a dimeric protein consisting of two polypeptides as defined in (ii); preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker,
for the manufacture of a medicament for the treatment of a subject suffering from an infectious disease or being in need of prevention thereof, wherein the medicament is administered to said subject.

The vaccine, the targeting unit, the multimerization/dimerization unit, the antigenic unit, the subunit comprising T cell epitopes, and the linkers are described in detail herein above.

Also disclosed herein is the use of a vaccine comprising:
(i) a polynucleotide comprising a nucleotide sequence encoding a polypeptide comprising: a targeting unit targeting or capable of targeting antigen-presenting cells; a multimerization unit such as a dimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker,
for the treatment of a subject suffering from an infectious disease or being in need of prevention thereof, wherein the medicament is administered to said subject.

Also disclosed herein is the use of a vaccine comprising:
(i) a polynucleotide comprising a nucleotide sequence encoding a polypeptide comprising: a targeting unit targeting or capable of targeting antigen-presenting cells; a multimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker,
for the treatment of a subject suffering from an infectious disease or being in need of prevention thereof, wherein the medicament is administered to said subject.

Also disclosed herein is the use of a vaccine comprising:
(i) a polynucleotide comprising a nucleotide sequence encoding a polypeptide comprising: a targeting unit targeting or capable of targeting antigen-presenting cells; a dimerization unit; and an antigenic unit comprising one or more T cell epitopes and one or more antigens or parts or fragments thereof; or
(ii) a polypeptide encoded by the nucleotide sequence defined in (i); or
(iii) a dimeric protein consisting of two polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker,
for the treatment of a subject suffering from an infectious disease or being in need of prevention thereof, wherein the medicament is administered to said subject.

The vaccine, the targeting unit, the multimerization/dimerization unit, the antigenic unit, the subunit comprising T cell epitopes, and the linkers are described in detail herein above.

Also disclosed herein is a vaccine comprising:
(i) a polynucleotide comprising a nucleotide sequence encoding a polypeptide comprising: a targeting unit targeting or capable of targeting antigen-presenting cells; a multimerization unit such as a dimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
preferably wherein the one or more T cell epitopes and the one or more antigens or parts or fragments thereof and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker,
when used in the treatment of an infectious disease.

Also disclosed herein is a vaccine comprising:
(i) a polynucleotide comprising a nucleotide sequence encoding a polypeptide comprising: a targeting unit targeting or capable of targeting antigen-presenting cells; a multimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker,
when used in the treatment of an infectious disease.

Also disclosed herein is a vaccine comprising:
(i) a polynucleotide comprising a nucleotide sequence encoding a polypeptide comprising: a targeting unit targeting or capable of targeting antigen-presenting cells; a dimerization unit; and an antigenic unit comprising one or more T cell epitopes and one or more antigens or parts or fragments thereof; or
(ii) a polypeptide encoded by the nucleotide sequence defined in (i); or
(iii) a dimeric protein consisting of two polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker,
when used in the treatment of an infectious disease.

The vaccine, the targeting unit, the multimerization/dimerization unit, the antigenic unit, the subunit comprising T cell epitopes, and the linkers are described in detail herein above.

Also disclosed is the use of a vaccine comprising:
(i) a polynucleotide comprising a nucleotide sequence encoding a polypeptide comprising: a targeting unit targeting or capable of targeting antigen-presenting cells; a multimerization unit such as a dimerization unit; and an antigenic unit comprising one or more T cell epitopes and one or more antigens or parts or fragments thereof, or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker,
for treating an infectious disease.

Also disclosed herein is the use of a vaccine comprising:
(i) a polynucleotide comprising a nucleotide sequence encoding a polypeptide comprising: a targeting unit targeting or capable of targeting antigen-presenting cells; a multimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker
for treating an infectious disease.

Also disclosed herein is the use of a vaccine comprising:
(i) a polynucleotide comprising a nucleotide sequence encoding a polypeptide comprising: a targeting unit targeting or capable of targeting antigen-presenting cells; a dimerization unit; and an antigenic unit comprising one or more T cell epitopes and one or more antigens or parts or fragments thereof; or
(ii) a polypeptide encoded by the nucleotide sequence defined in (i); or
(iii) a dimeric protein consisting of two polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker
for treating an infectious disease.

The vaccine, the targeting unit, the multimerization/dimerization unit, the antigenic unit, the subunit comprising T cell epitopes, and the linkers are described in detail herein above.

Also disclosed herein is a medicament for the treatment of an infectious disease in a subject having said infectious disease by administering to the subject a vaccine comprising:
(i) a polynucleotide comprising a nucleotide sequence encoding a polypeptide comprising: a targeting unit targeting or capable of targeting antigen-presenting cells; a multimerization unit such as a dimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker.

Also disclosed herein is a medicament for the treatment of an infectious disease in a subject having said infectious disease by administering to the subject a vaccine comprising:
(i) a polynucleotide comprising a nucleotide sequence encoding a polypeptide comprising: a targeting unit targeting or capable of targeting antigen-presenting cells; a multimerization unit; and an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the multimerization unit by a unit linker and separated from the one or more antigens or parts or fragments thereof by an antigen linker.

Also disclosed herein is a medicament for the treatment of an infectious disease in a subject having said infectious disease by administering to the subject a vaccine comprising:
(i) a polynucleotide comprising a nucleotide sequence encoding a polypeptide comprising: a targeting unit targeting or capable of targeting antigen-presenting cells; a dimerization unit; and an antigenic unit comprising one or more T cell epitopes and one or more antigens or parts or fragments thereof; or
(ii) a polypeptide encoded by the nucleotide sequence defined in (i); or
(iii) a dimeric protein consisting of two polypeptides as defined in (ii);
preferably wherein the one or more T cell epitopes and the one or more antigens or parts or fragments thereof are from a pathogen;
wherein the antigenic unit comprises a subunit comprising the T cell epitopes which are separated from each other by T cell linkers, if more than one T cell epitope is comprised in the subunit; and wherein the subunit is connected to the dimerization unit by a unit linker and separated from the one or more antigens or parts or fragments thereof by an antigen linker.

The vaccine, the targeting unit, the multimerization/dimerization unit, the antigenic unit, the subunit comprising T cell epitopes, and the linkers are described in detail herein above.

### EXAMPLES

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims

### Example 1: Selection of T Cell Epitopes for Inclusion into a Construct of the Disclosure for Use as a Vaccine Against Infection with SARS-CoV-2

The predicted immunogenic epitopes from conserved regions of SARS CoV viruses were identified as follows:

In a first step, the worldwide population of HLA class I and II alleles were identified. For HLA class I, the allele frequency database available at http://www.allelefrequencies.net was used and the identification of the most frequent HLA alleles was conducted in the following manner:

A separate search for each locus: A, B and C and a separate regional search for Europe, South-East Asia (focus on China) and North America (focus on USA) was carried out. The population standard was set to “Gold” to obtain only the high-quality studies. The level of resolution was set to at least 4 digits, for instance: HLA-A*01:01. The sampling year was set to 2005 and later. The top 4 frequent alleles for each study were collected. Among all top 4 frequent alleles for all studies, the top 4 or 5 frequent alleles for each region (Europe/South-East Asia/North America) was selected. Due to overlap between the regions, the number of the final selected alleles was 10, 10 and 11 for A, B and C, respectively. These 31 HLA class I alleles cover 99.4% of the world population, as estimated by IEDB population coverage estimation tool (http://tools.iedb.org/population/). The coverage in detail was as follows: Europe: 99.9%; North America: 99.2%; South America: 92.7%; East Asia: 98.5%; Southeast Asia: 98.1%; Northeast Asia: 97.4%; South Asia: 93.1%; Southwest Asia: 93.3%; Central Africa: 94.3%; East Africa: 92.3%; North Africa: 96.2%; South Africa: 91.2% and West Africa: 94.3%.

For HLA class II, although not done in this Example 1, the allele frequency may be identified in a similar manner as for HLA class I.

In a next step, T cell epitopes for SARS-CoV-2 were identified as follows: high-quality SARS-CoV-2 reference amino-acid sequences were obtained. The annotated (annotation score 5/5) Uniprot Wuhan strain was downloaded from Uniprot, query SARS-CoV-2 (https://www.uniprot.org/uniprot/?query=sars-cov-2&fil=organism%3A%22Severe%20acute%20respiratory%20syndrome%20coronavirus%202%20(2019-nCoV)%20(SARS-CoV-2)%20%5B2697049%5D%22&columns=id%2Centry%20name%2Creviewed%2Cprotein%20names%2Cgenes%2Corganism%2Clength&sort=score). Six proteins were selected: four structural proteins, the spike protein, the envelope protein, the membrane protein, and the nucleocapsid protein and two non-structural proteins, ORF1a/b and ORF3a. A search was carried out for hotspot genomic areas of epitopes predicted to bind to HLA class I alleles in the six protein sequences using NetMHCpan 4.0 (https://services.healthtech.dtu.dk/service.php?NetNMCpan-4.0) and the HLA class I alleles as defined in the initial step. A total of 13236 epitopes predicted to bind to at least one HLA class I allele were found. To identify the hotspot areas, filtering was applied remove all epitopes that did not bind to more than 10 different HLA class I alleles and to at least 1 allele from each locus (A/B/C). The remaining 604 epitopes were further processed by merging the overlapping or adjacent epitopes (within 3 amino acids apart) to obtain hotspot epitope regions. Epitopes shorter than 15 amino acids were extended to 15 amino acids. The binding of the epitopes to HLA I and HLA II alleles was predicted using NetMHCpan 4.0 and NetMHCIIpan 3.2 (https://services.healthtech.dtu.dk/service.php?NetMHCIIpan-3.2), respectively, on the final list of merged epitopes.

Up-to-date high-quality annotated sequences from the NCBI virus database for SARS-CoV-2 and SARS-CoV were then obtained. The homology to these sequences by global alignment (% identity between the strain and epitope sequence) was determined. Up-to-date high-quality annotated human reference protein sequences were obtained from https://www.uniprot.org/proteomes/UP000005640. A summary of all identical matches between the epitopes and the human proteome using 6, 7, 8 and 9 amino acid short sequences was created and a search for substring matches between the epitopes and all epitopes deposited in the Immune Epitope Database (IEDB) shown to elicit T/B cell response or binding to MHC class I was carried out. The final prioritization of epitopes was made based on the collected information:
Maximizing the global population coverage by prioritizing epitopes covering a large set of distinct MHC class I and II alleles
Conservation within >200 different SARS CoV-2 strains worldwide from up-to-date NCBI virus database
Conservation within different SARS CoV strains and betacoronaviruses in general
No/minimal numbers of exact match of 6 amino acids to any protein in the human proteome
Identity with an immunodominant SARS-CoV epitope deposited in the Immune Epitope Database (IEDB)
   Over 400 T cell epitopes were identified and 3 of them were selected for inclusion into the antigenic unit (sequences described in Example 2).

### Example 2: Design and Production of DNA Constructs of the Disclosure for Use as a Vaccine Against Infection with SARS-CoV-2

All gene sequences of tested constructs (VB10.COV2) were ordered from Genscript (860 Centennial Ave., Piscataway, NJ 08854, USA) and cloned into the expression vector pUMVC4a.

Seventeen DNA constructs (in the following also called VB10.COV2) were designed. All constructs comprise an hMIP-1α targeting unit, a dimerization unit and as the antigen a part of the RBD of the spike protein of SARS-CoV-2. All but one construct, which was used as a comparison (VB2060), comprise one or more of the following T cell epitopes which were selected based on their predicted binding to HLA alleles as described in Example 1 (Table 1):

TABLE 1
selected T cell epitopes

| T cell | | SEQ |
|---|---|---|
| epitope | Sequence | ID NO: |
| pep08 | HFAIGLALYYPSARIVYTACSHAAV | 129 |
| pep18 | FVNEFYAYLRKHFSMM | 130 |
| pep25 | LVKPSFYVYSRVKNL | 131 |

VB2081 (encoding amino acid sequence SEQ ID NO: 4), comprising a subunit with 1 predicted T cell epitope, pep08 (SEQ ID NO: 129), and a (GGGGS)2 antigen linker (SEQ ID NO: 24, wherein m=2), which connects the subunit to the antigen.

VB2082 (encoding amino acid sequence SEQ ID NO: 5), comprising a subunit with 1 predicted T cell epitope, pep08 (SEQ ID NO: 129), and a (GGGGS)2 antigen linker (SEQ ID NO: 24, wherein m=2), which connects the subunit to the antigen.

VB2083 (encoding amino acid sequence SEQ ID NO: 6), comprising a subunit with 2 predicted T cell epitopes, pep08 (SEQ ID NO: 129) and pep18 (SEQ ID NO: 130), separated by a (GGGGS)2 T cell epitope linker (SEQ ID NO: 24, wherein m=2) and a (GGGGS)2 antigen linker (SEQ ID NO: 24, wherein m=2), which connects the subunit to the antigen.

VB2084 (encoding amino acid sequence SEQ ID NO: 7), comprising a subunit with 3 predicted T cell epitopes, pep08 (SEQ ID NO: 129), pep18 (SEQ ID NO: 130) and pep25 (SEQ ID NO: 131), separated by (GGGGS)2 T cell epitope linkers (SEQ ID NO: 24, wherein m=2) and a (GGGGS)2 antigen linker (SEQ ID NO: 24, wherein m=2), which connects the subunit to the antigen.

VB2085 (encoding amino acid sequence SEQ ID NO: 8), comprising a subunit with 1 predicted T cell epitope, pep08 (SEQ ID NO: 129), and a GLGGL antigen linker (SEQ ID NO: 42), which connects the subunit to the antigen.

VB2086 (encoding amino acid sequence SEQ ID NO: 9), comprising a subunit with 1 predicted T cell epitope, pep08 (SEQ ID NO: 129), and a (GLGGL)2 antigen linker (SEQ ID NO: 90, wherein m=2), which connects the subunit to the antigen.

VB2087 (encoding amino acid sequence SEQ ID NO: 10), comprising a subunit with 1 predicted T cell epitope, pep18 (SEQ ID NO: 130), and a GLGGL antigen linker (SEQ ID NO: 42), which connects the subunit to the antigen.

VB2088 (encoding amino acid sequence SEQ ID NO: 11), comprising a subunit with 2 predicted T cell epitopes, pep08 (SEQ ID NO: 129) and pep18 (SEQ ID NO: 130), separated by a (GGGGS)2 T cell epitope linker (SEQ ID NO: 131) and a GLGGL antigen linker (SEQ ID NO: 42), which connects the subunit to the antigen.

VB2089 (encoding amino acid sequence SEQ ID NO: 12), comprising a subunit with 3 predicted T cell epitopes, pep08 (SEQ ID NO: 129), pep18 (SEQ ID NO: 130) and pep25 (SEQ ID NO: 131), separated by (GGGGS)2 T cell epitope linkers (SEQ ID NO: 24, wherein m=2) and a GLGGL antigen linker (SEQ ID NO: 42), which connects the subunit to the antigen.

VB2091 (encoding amino acid sequence SEQ ID NO: 13), comprising a subunit with 1 predicted T cell epitope, pep08 (SEQ ID NO: 129), and a TQKSLSLSPGKGLGGL antigen linker (SEQ ID NO: 78), which connects the subunit to the antigen.

VB2092 (encoding amino acid sequence SEQ ID NO: 14), comprising a subunit with 3 predicted T cell epitopes, pep08 (SEQ ID NO: 129), pep18 (SEQ ID NO: 130) and pep25 (SEQ ID NO: 131), separated by (GGGGS)2 T cell epitope linkers (SEQ ID NO: 24, wherein m=2) and a TQKSLSLSPGKGLGGL antigen linker (SEQ ID NO: 78), which connects the subunit to the antigen.

VB2094 (encoding amino acid sequence SEQ ID NO: 15), comprising a subunit with 1 predicted T cell epitope, pep08 (SEQ ID NO: 129), and a SLSLSPGKGLGGL antigen linker (SEQ ID NO: 79), which connects the subunit to the antigen.

VB2095 (encoding amino acid sequence SEQ ID NO: 16), comprising a subunit with 3 predicted T cell epitopes, pep08 (SEQ ID NO: 129), pep18 (SEQ ID NO: 130) and pep25 (SEQ ID NO: 131), separated by (GGGGS)2 T cell epitope linkers (SEQ ID NO: 24, wherein m=2) and a SLSLSPGKGLGGL antigen linker (SEQ ID NO: 79), which connects the subunit to the antigen.

VB2097 (encoding amino acid sequence SEQ ID NO: 17), comprising a subunit with 3 predicted T cell epitopes, pep08 (SEQ ID NO: 129), pep18 (SEQ ID NO: 130) and pep25 (SEQ ID NO: 131), separated by (GGGGS)2 T cell epitope linkers (SEQ ID NO: 24, wherein m=2) and a GGSAGGSGSGSSGGSSGASGTGTAGGTGSGSGTGSG antigen linker (SEQ ID NO: 80), which connects the subunit to the antigen.

VB2099 (encoding amino acid sequence SEQ ID NO: 18), comprising a subunit with 3 predicted T cell epitopes, pep08 (SEQ ID NO: 129), pep18 (SEQ ID NO: 130) and pep25 (SEQ ID NO: 131), separated by (GGGGS)2 T cell epitope linkers (SEQ ID NO: 24, wherein m=2) and a GGSGGGSEGGGSEGGGSEGGGSEGGGSEGGGSGGGS antigen linker (SEQ ID NO: 81), which connects the subunit to the antigen.

### Example 3: In Vitro Characterization of the Protein Expression and Secretion Post Transient Transfection of Mammalian Cells with the DNA Constructs

The purpose of this study was to characterize the VB10.COV2 protein expression and secretion post transient transfection of mammalian cells with the VB10.COV2 DNA plasmids, by measuring the presence of VB10.COV2 proteins in the cell supernatant by an ELISA assay using binding of specific antibodies to the targeting, dimerization, and antigenic units of the proteins.

The VB10.COV2/Combo DNA constructs were synthesized, cloned and produced by Genscript. The resulting constructs encoded for homodimeric proteins with MIP-1α targeting unit, T cell epitopes/part of RBD as antigenic unit, and a dimerization unit consisting of human hinge exons h1 and h4 and CH3 domain of IgG3. Genscript also performed DNA plasmid preparation (0.5-1.0 mg). The plasmid DNA sequence was verified by sanger sequencing, plasmid identity and supercoil content was verified by restriction digest and agarose gel electrophoresis (Genscript).

HEK293 cells were obtained from ATCC. HEK293 cells were transiently transfected with VB10.COV2 DNA plasmids. Briefly, 2×105 cells/well were plated in 24-well tissue culture plates with 10% FBS growth medium and transfected with 1 μg VB10.COV2 DNA plasmid using Lipofectamine® 2000 reagent under the conditions suggested by the manufacturer (Invitrogen, Thermo Fischer Scientific). The transfected cells were then maintained for 6 days at 37° C. with 5% CO₂ and the cell supernatant was harvested for characterization of the VB10.COV2 protein expression.

ELISA was performed to verify the amount of VB10.COV2 protein produced by the HEK293 cells and secreted into the cell supernatant. MaxiSorp Nunc-immuno plates were coated with 1 μg/ml of anti hIgG (hIgG contains the CH3 domain of the dimerization unit included in the constructs) as capture antibody (MCA878G, BioRad) in 1×PBS with 100 μl/well and plates were incubated overnight at 4° C. The microtiter wells were blocked by the addition of 200 μl/well 4% BSA in 1×PBS. 100 μl of cell supernatant from transfected HEK293 cells containing VB10.COV2 proteins were added to the plates. For detection antibody, SARS-CoV-2/2019-nCoV RBD antibody (1:1000) (Sino Biological) was added and incubated. Thereafter, anti-Rabbit IgG-HRP (1:5000) was added and incubated. Unless specified, all incubations were carried out at 37° C. for 1 h, followed by 3× washing with PBS-Tween. Afterwards, 100 μl/well of TMB solution was added and color development was stopped after 5-15 min adding 100 μl/well of 1 M HCl. The optical density at 450 nm was determined on an automated plate reader (Thermo Scientific Multiscan GO).

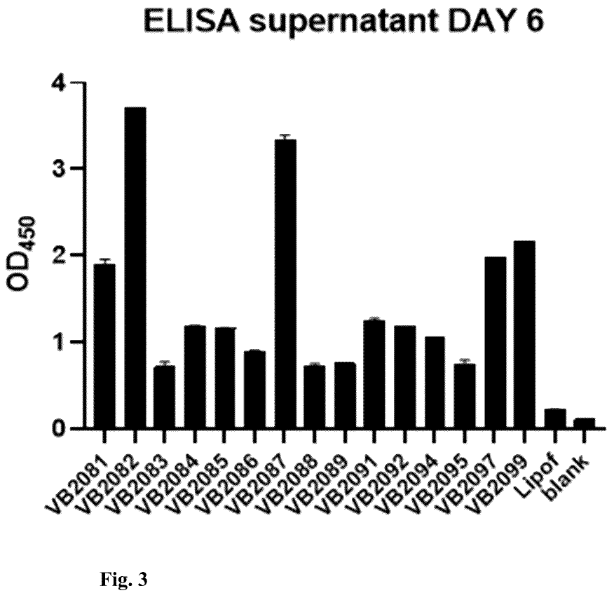

FIG. 3 shows that all constructs were expressed and secreted as proteins, although the secretion level varies and depends on the included T cell epitopes and/or the antigen linker. The secretion levels were higher for constructs containing pep18 (VB2082 and VB2087) compared to constructs containing pep08 (VB2081). When constructs comprised 3 T cell epitopes (pep08, pep18 and pep25), those with a longer antigen linker sequence such as SEG or GSAT were significantly better expressed compared to the constructs with other, shorter antigen linkers.

In conclusion, Example 3 shows successful protein expression and secretion of the constructs in HEK293 cells.

### Example 4: Anti-RBD Immune Responses in Mice Immunized with Constructs of the Disclosure

For all experiments with mice (Examples 4-5), the following study design was applied:

Female, 6-8-week-old BALB/c mice were obtained from Janvier Labs (France). All animals were housed in the animal facility at the Radium Hospital (Oslo, Norway) or the University of Oslo (Norway). All animal protocols were approved by the Norwegian Food Safety Authority (Oslo, Norway). For the studies, the mice were vaccinated with the DNA constructs as follows:

Mice received on day 0 1 dose of a vaccine comprising 25 μg p DNA Combo construct (DNA plasmid) dissolved in sterile PBS. The vaccine was administrated to each tibialis anterior (TA) muscle by needle injection of 25 μl of the vaccine followed by AgilePulse in vivo electroporation (EP) (BTX, U.S.). The AgilePulse EP delivery consists of 3 sets of pulses with 110-450 voltage. The first set, there are 1 50 μs pulse with a 0.2 ms delay; the second set is 1 50 μs pulse with a 50 ms delay and the third set is 8 pulses with 10 ms pulse and 20 ms delay. Sera were collected on day 14 and tested for anti-RBD IgG antibodies binding the RBD protein.

The purpose of the study was to evaluate the humoral immune response induced in mice against RBD when vaccinated with the constructs.

The humoral immune response was evaluated in sera collected from vaccinated mice by an ELISA assay detecting total IgG in the sera binding to RBD from SARS-CoV2. Nunc ELISA plates were coated with 1 μg/ml recombinant protein antigen in PBS overnight at 4° C. Plates were blocked with 4% BSA in PBS for 1 hour at RT. Plates were then incubated with serial dilutions of mouse sera and incubated for 2 h at 37° C. Plates were washed 3× and incubated with 1:50 000 dilution of HRP-anti-mouse IgG secondary antibody (Southern Biotech) and incubated for 1 h at 37° C. After final washing, plates were developed using TMB substrate (Merck, cat. CL07-1000). Plates were read at 450 nm wavelength within 30 min using a Multiscan GO (Thermo Fischer Scientific). Binding antibody endpoint titers were calculated. Binding antigens tested included SARS-CoV-2 antigens: RBD (Sino Biological 40592-V08H; (SEQ ID NO: 142).

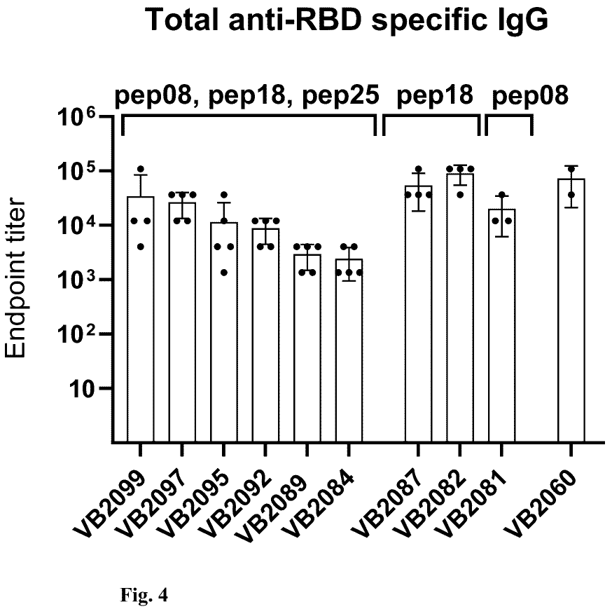

As shown in FIG. 4, constructs VB2097 (3 T cell epitopes/GSAT antigen linker) and VB2099 (3 epitopes/SEG antigen linker) induced stronger IgG responses against RBD than the constructs comprising 3 epitopes with other linkers. As for the constructs with 1 epitope, VB2082 and VB2087 (including pep18) induced stronger anti-RBD IgG responses compared to constructs comprising the pep08 epitope (VB2081) Moreover, two of the constructs containing a combination of predicted T cell epitopes and a part of RBD, VB2097 and VB2087 induce similar immune responses compared to VB2060, comprising only said part of the RBD and no T cell epitopes.

### Example 5: Induction of Specific Cellular Responses to T Cell Epitopes and RBD by Vaccination with Constructs According to the Disclosure

The purpose of this study was to evaluate the cellular immune response against both the predicted T cell epitopes and the RBD in splenocytes from mice vaccinated with the constructs.

Splenocytes from vaccinated mice were analyzed in an IFN-γ-ELISpot assay detecting predicted T cell epitopes and RBD-specific cellular responses. Briefly, the animals were sacrificed at day 14 and the spleens were harvested aseptically. The spleens were mashed, cell suspensions were incubated with 1×ACK buffer, washed and re-suspended to a cell concentration of 6×10⁵ cells. Furthermore, the cells were plated in triplicates (6×10⁵ cells/well) and stimulated with 2 μg/ml of individual peptides (T cell epitopes included in the respective constructs) and 2 μg/ml of RBD peptide pools (Table 2) for 24 h. The RBD peptide pools comprised 15-mer peptides overlapping by 12 amino acids spanning regions of the RBD.

TABLE 2

| Pool ID | Composition |
|---|---|
| RBD pool 2 | RBD 11, 12, 13, 14, 15, 16, 17, 18, 19, 24 |
| RBD pool 6 | RBD 52, 53, 54, 55, 56, 57, 58, 59, 60, 61 |

No-peptide-stimulation was used as negative control. The stimulated splenocytes were analyzed for IFN-γ responses using the IFN-γ ELISpot Plus kit (Mabtech AB, Sweden). Spot-forming cells were measured in an IRIS Fluorospot and ELISpot plate reader (Mabtech AB) and analyzed using the Apex software (Mabtech AB) Results are shown as the mean number of IFN-γ+ spots/10⁶ splenocytes.

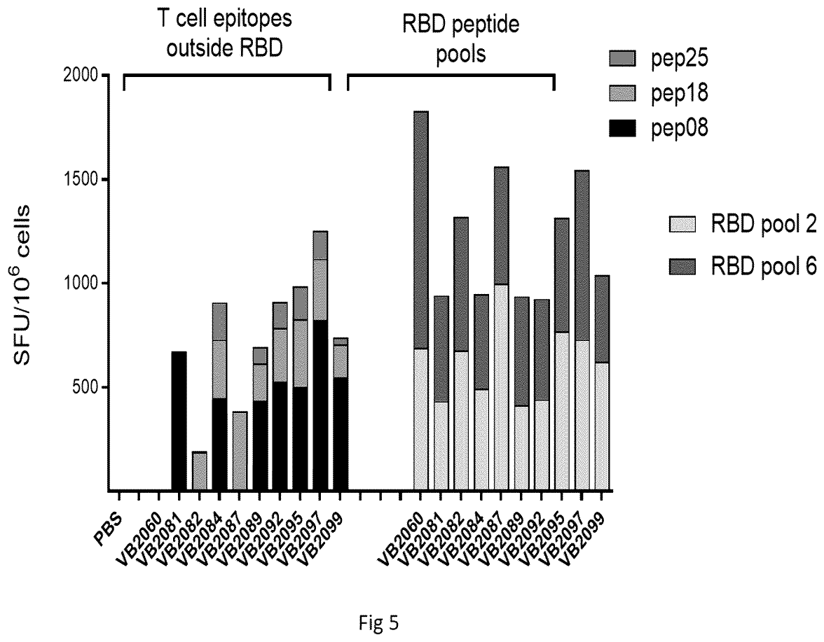

As shown in FIG. 5, strong T cell responses against the predicted T cell epitopes from multiple SARS-CoV-2 strains were detected in spleens at day 14 from mice vaccinated once with 25 μg of constructs dissolved in sterile PBS (as described in Example 4) containing either one or three predicted T cell epitopes. VB2097 (3 T cell epitopes/GSAT antigen linker) induced a stronger T cell specific response (˜1250 SFC per 10⁶ cells) than the other constructs comprising 3 epitopes with other linkers. In addition, all constructs were also able to elicit a strong RBD-specific cellular response. VB2097 and VB2087 elicited the strongest responses against RBD at a similar level to the response elicited by VB2060 (FIG. 5).

### Conclusion

It has been shown that eliciting both strong RBD-specific antibody and T cell responses in addition to specific T cell responses against predicted T cell epitopes from the SARS-COV2 genome is feasible by including the predicted T cell epitopes with a part of RBD in the antigenic unit in the construct of the disclosure. These findings, together with simple administration and storage stability even at elevated temperatures (data not shown), suggest that the constructs are promising future candidates to prevent and treat Covid-19.

### Example 6: Design and Production of DNA Constructs of the Disclosure for Use as a Vaccine Against Infection with SARS-CoV-2

All gene sequences of tested constructs were ordered from Genscript (Genscript Biotech B.V., Netherlands) and cloned into the expression vector pUMVC4a; a master plasmid comprising a nucleotide sequence encoding the signal peptide, targeting unit, dimerization unit and unit linker described in Table 3.

DNA plasmids TECH004-IV002 (SEQ TD NO: 169), TECH004-V003 (SEQ ID NO: 170), TECH004-IV004 (SEQ ID NO: 171) and TECH004-V005 (SEQ ID NO: 172) comprise nucleic acid sequences encoding elements/units including a targeting unit, a dimerization unit, and a unit linker (Table 3) followed by an antigen unit comprising one or more of the T cell epitopes (Table 4), an antigen linker and the RBD (amino acids 319-542) of the spike protein of SARS-CoV-2 (Wuhan variant).

TABLE 3

| Elements/Units | Description and sequence |
|---|---|
| Signal peptide | Native leader sequence for human CCL3L1 |
| | Amino acids 1-23 of SEQ ID NO: 1 |
| Targeting unit | Full length human CCL3L1 sequence |
| | Amino acids 24-93 of SEQ ID NO: 1 |
| Dimerization | Human hinge-region 1 from IgG |
| unit | Amino acids 94-105 of SEQ ID NO: 1 |
| | Human hinge-region 4 from IgG3 |
| | Amino acids 106-120 of SEQ ID NO: 1 |
| | Glycine-serine-linker |
| | Amino acids 121-130 of SEQ ID NO: 1 |
| | Human CH3 domain from IgG3 |
| | Amino acids 131-237 of SEQ ID NO: 1 |
| Unit linker | Glycine-leucine linker |
| | Amino acids 238-242 of SEQ ID NO: 1 |

TABLE 4

| T cell | | SEQ |
|---|---|---|
| epitope | Sequence | ID NO: |
| Pep01 | RSFIEDLLFNKVTLA | 142 |
| pep02 | MTYRRLISMMGFKMNYQVNGYPNMF | 143 |
| pep03 | LMIERFVSLAIDAYP | 144 |
| pep04 | RAMPNMLRIMASLVL | 145 |
| pep05 | MVYMPASWVMRIMTW | 146 |
| pep06 | FLNRFTTTLNDFNLVAM | 147 |
| pep07 | SSVELKHFFFAQDGNAAI | 148 |
| pep08 | HFAIGLALYYPSARIVYTACSHAAV | 129 |
| pep09 | YFIKGLNNLNRGMVL | 149 |
| pep10 | YLNTLTLAVPYNMRV | 150 |
| pep11 | AQFAPSASAFFGMSRI | 151 |
| pep12 | EIVDTVSALVYDNKL | 152 |
| pep13 | SSGDATTAYANSVFNICQAVTANVNALL | 153 |
| pep14 | HVISTSHKLVLSVNPYV | 154 |
| pep15 | MLSDTLKNLSDRVVFVLWAHGFEL | 155 |
| pep17 | ASIKNFKSVLYYQNNVFM | 156 |
| pep18 | FVNEFYAYLRKHFSMM | 130 |
| pep19 | RVWTLMNVLTLVYKV | 157 |
| pep20 | FAYANRNRFLYIIKL | 158 |
| pep25 | LVKPSFYVYSRVKNL | 131 |

TECH004-IV002 (encoding amino acid sequence SEQ ID NO: 169), comprising a subunit with 3 predicted T cell epitopes, pep08 (SEQ ID NO: 129), pep18 (SEQ ID NO: 130) and pep25 (SEQ ID NO: 131), separated by (GGGGS)2 T cell epitope linkers (SEQ ID NO: 24, wherein m=2) and a GGSAGGSGSGSSGGSSGASGTGTAGGTGSGSGTGSG antigen linker (SEQ ID NO: 80), which connects the subunit to the RBD antigen (SEQ ID NO: 125).

TECH004-IV003 (encoding amino acid sequence SEQ ID NO: 170), comprising a subunit with 5 predicted T cell epitopes, pep08 (SEQ ID NO: 129), pep18 (SEQ ID NO: 130), pep09 (SEQ ID NO: 149), pep13 (SEQ ID NO: 153) and pep25 (SEQ ID NO: 131), separated by (GGGGS)2 T cell epitope linkers (SEQ ID NO: 24, wherein m=2) and a GGSAGGSGSGSSGGSSGASGTGTAGGTGSGSGTGSG antigen linker (SEQ ID NO: 80), which connects the subunit to the RBD antigen (SEQ ID NO: 125).

TECH004-IV004 (encoding amino acid sequence SEQ ID NO: 171), comprising a subunit with 10 predicted T cell epitopes, pep02 (SEQ ID NO: 143), pep09 (SEQ ID NO: 149), pep08 (SEQ ID NO: 129), pep05 (SEQ ID NO: 146), pep20 (SEQ ID NO: 158), pep06 (SEQ ID NO: 147), pep07 (SEQ ID NO: 148), pep13 (SEQ ID NO: 153), pep18 (SEQ ID NO: 130), pep 25 (SEQ ID NO: 131), separated by (GGGGS)2 T cell epitope linkers (SEQ ID NO: 24, wherein m=2) and a GGSAGGSGSGSSGGSS GASGTGTAGGTGSGSGTGSG antigen linker (SEQ ID NO: 80), which connects the subunit to the RBD antigen (SEQ ID NO: 125).

TECH004-IV005 (encoding amino acid sequence SEQ ID NO: 172), comprising a subunit with 20 predicted T cell epitopes, pep02 (SEQ ID NO: 143), pep14 (SEQ ID NO: 154), pep17 (SEQ ID NO: 156), pep09 (SEQ ID NO: 149), pep10 (SEQ ID NO: 150), pep15 (SEQ ID NO: 155), pep08 (SEQ ID NO: 129), pep01 (SEQ ID NO: 142), pep03 (SEQ ID NO: 144), pep04 (SEQ ID NO: 145), pep05 (SEQ ID NO: 146), pep19 (SEQ ID NO: 157), pep20 (SEQ ID NO: 158), pep 11 (SEQ ID NO: 151), pep12 (SEQ ID NO: 152), pep06 (SEQ ID NO: 147), pep07 (SEQ ID NO: 148), pep13 (SEQ ID NO: 153), pep18 (SEQ ID NO: 130), pep25 (SEQ ID NO: 131), separated by (GGGGS)₂ T cell epitope linkers (SEQ ID NO: 24, wherein m=2) and a GGSAGGSGSGSSGGSSGASGTGTA GGTGSGSGTGSG antigen linker (SEQ ID NO: 80), which connects the subunit to the RBD antigen (SEQ ID NO: 125).

### Example 7: In Vitro Assessment of Expression and Secretion of Proteins Encoded by TECH004-IV002, TECH004-IV003, TECH004-IV004, and TECH004-IV005

The purpose of this study was to characterize the protein expression and secretion post transient transfection of mammalian cells with the TECH004-IV002, TECH004-IV003, TECH004-IV004 and TECH004-IV005 DNA plasmids by measuring the presence of proteins in the cell supernatant by an ELISA assay using antibodies detecting the targeting and dimerization units. In addition, western blot (WB) analysis was performed on supernatant samples from transfected Expi293F cells to further characterize the proteins encoded by TECH004-IV002, TECH004-IV003, TECH004-IV004, and TECH004-IV005.

Briefly, Expi293F cells (2×10⁶ cells/ml, 1 ml) were seeded in a 96-well culture plate. The cells were transfected with 0.64 μg/ml plasmid DNA using ExpiFectamine 293 Reagent (100014994 Thermo Fisher Sci.), and the plates were incubated on an orbital shaker (3 mm diameter, 900 rpm) in a humidified CO₂ cell incubator (8% CO₂, 37° C.). The plates were incubated for 72 h before the supernatant was harvested.

The secreted proteins were characterized in a sandwich ELISA of the supernatant using mouse anti human IgG CH3 domain antibody (capture antibody, 100 μl/well, 2 μg/ml, MCA878G, Bio-Rad), rabbit anti-SARS-CoV-2/2019-nCoV RBD antibody (detection antibody, 100 μl/well, 40592-T62, Sino Biological), and goat anti-rabbit IgG-HRP antibody (secondary HRP-conjugated antibody, 100 μl/well, 0.16 μg/ml, 31460, Thermo Fisher Scientific).

Further, the samples were prepared for SDS-PAGE by mixing 70 μl supernatant from transfected Expi293F cells with 25 μl 4× Laemmli sample buffer (Bio-Rad) with 5 μl DTT (Thermo Fisher Sci.) for reducing conditions. The samples were heated at 70° C. for 10 minutes (min) before adding 20 μL per lane to 4%-20% Criterion TGX Stain-Free precast gels (Bio-Rad). SDS-PAGE was performed in 1× Tris/Glycine/SDS running buffer (Bio-Rad) with Precision Plus Protein All Blue Prestained and Unstained protein standards (Bio-Rad). Proteins were transferred from the gel onto EtOH activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with goat anti-human MIP-1α (AF270, R&D Systems). The membranes were washed, incubated with fluorochrome-conjugated secondary antibodies for 1 h at room temperature, and then washed and dried (rinsed in ethanol). Images were acquired by using a ChemiDoc™ MP Imaging System (setting Dylight 650 and 800, Auto Optimal).

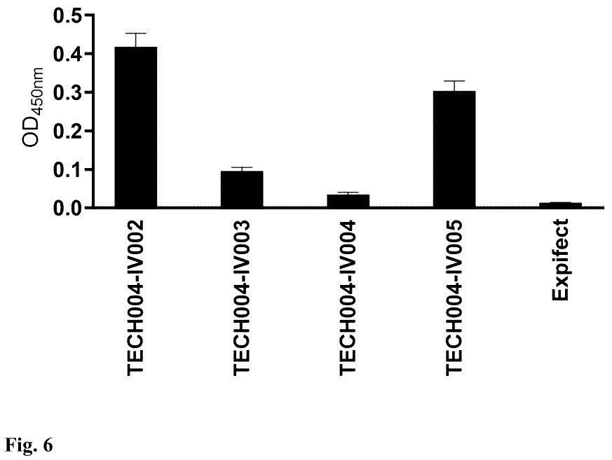

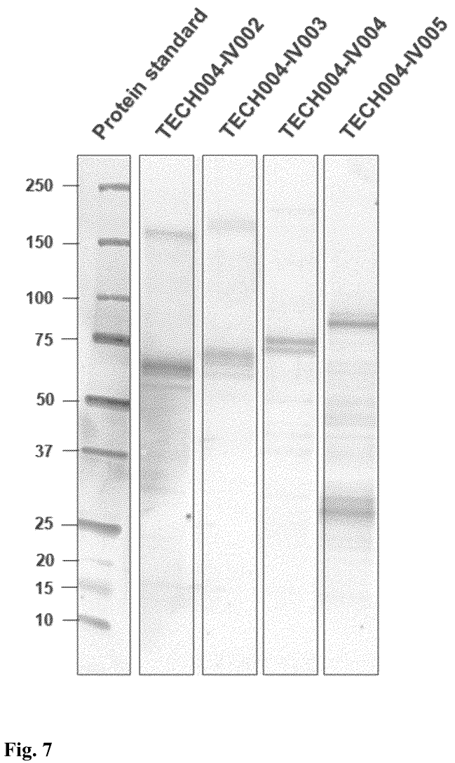

FIG. 6 shows that all constructs were expressed and secreted as proteins from transfected Expi293F cells, although the expression level varies and depends on the included T cell epitopes. The western blot analysis demonstrated that TECH004-IV002, TECH004-IV003, TECH004-IV004, and TECH004-IV005 all are expressed as full-length proteins (FIG. 7). Also, the lane loaded with sample from TECH004-IV005 showed both full-length protein of the monomeric band at 75 kDa (reducing conditions) and possibly truncated or cleaved product band at 30 kDa.

### Conclusion

Taken together, the ELISA and western blot data demonstrate full-length expression of the Combo proteins with an antigenic unit comprising the same antigen but varying subunits, in which the subunit comprises varying numbers and lengths of T cell epitopes.

### Example 8: Assessment of T Cell Response Induced Against T Cell Epitopes and RBD (Aa 319-542) from SARS-COV-2 in TECH004-IV002, TECH004-IV003, TECH004-IV004, and TECH004-IV005

Female, 6-week-old BALB/c mice were obtained from Janvier Labs (France). All animals were housed in the animal facility at the University of Oslo (Oslo, Norway). All animal protocols were approved by the Norwegian Food Safety Authority (Oslo, Norway). 5 mice/group were used for the testing of the constructs comprising an antigenic unit, whereas 3 mice/group were used for the negative control.

Immunogenicity of TECH004-IV002, TECH004-IV003, TECH004-IV004, and TECH004-IV005 was determined; VB1026 was used as negative control. VB1026 encoding the polypeptide with amino acid sequence of 1-237 of SEQ ID NO: 1, was included. This DNA plasmid is identical to TECH004-IV002, TECH004-IV003, TECH004-IV004, and TECH004-IV005, but comprises no antigenic unit.

A final dose of 25 μg DNA plasmid dissolved in sterile PBS was administered by intramuscular needle injection to each tibialis anterior (2×25 μl, 500 μg/ml), followed by electroporation with AgilePulse in vivo electroporation system (BTX, USA).

The spleens were collected 13 days after vaccination and mashed in a cell strainer to obtain a single cell suspension. The red blood cells were lysed using ammonium-chloride-potassium (ACK) lysing buffer. The splenocytes were pooled with in respective vaccinated groups and counted using the NucleoCounter NC-202 (ChemoMetec, Denmark) and resuspended to a final concentration of 6×10⁶ cells/ml. The splenocytes were then seeded at 6×10⁵ cells/well and re-stimulating with 4 μg/ml of single peptides corresponding to T cell epitopes (Table 4) and 2 μg/ml RBD peptide pools (Table 5) for 24 hours. No-peptide-stimulation was used as negative control. The stimulated splenocytes were analyzed for IFN-γ responses using the IFN-γ FluoroSpot kit (Mabtech AB, Sweden). Spot-forming cells were measured in an IRIS Fluorospot and ELISpot plate reader (Mabtech AB) and analyzed using the Apex software (Mabtech AB).

TABLE 5

| Pool ID | Composition |
|---|---|
| Pool 1 | RBD-1, 2, 3, 4, 5, 6, 7, 8, 9, 10 |
| Pool 2 | RBD-11, 12, 13, 14, 15, 16, 17, 18, 19, 24 |
| Pool 3 | RBD-20, 21, 22, 23, 25, 26, 27, 28, 29, 30 |
| Pool 4 | RBD-31, 32, 33, 34, 36, 37, 38, 39, 40 |
| Pool 5 | RBD-41, 42, 43, 44, 45, 46, 47, 48, 49, 50, 51 |
| Pool 6 | RBD-52, 53, 54, 55, 56, 57, 58, 59, 60, 61 |

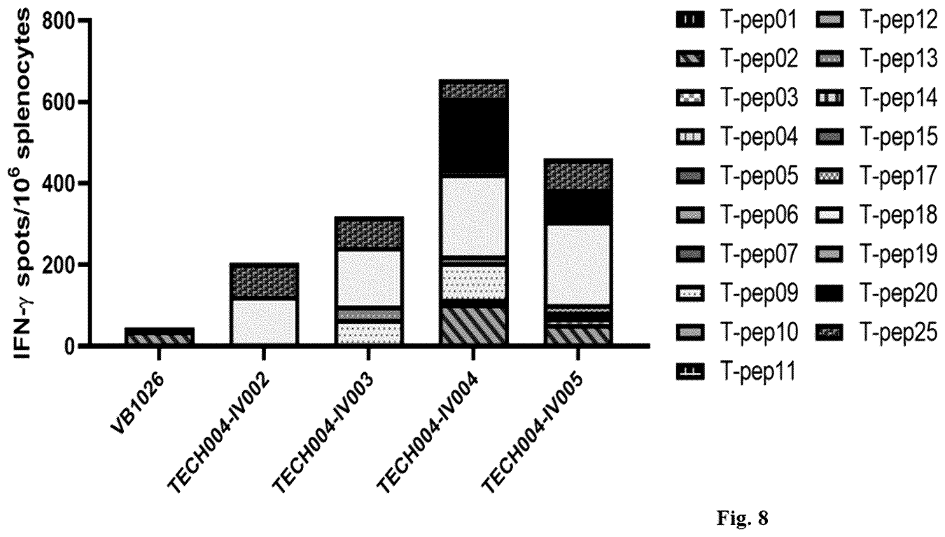

T cell responses against the predicted T cell epitopes from multiple SARS-CoV-2 strain were detected in spleens at day 13 from mice vaccinated once with 25 μg of constructs containing either 3, 5, 10, or 20 predicted T cell epitopes (FIG. 8). In addition, all constructs were also able to elicit a Wuhan RBD-specific T cell response (FIG. 9). Results are shown as the mean number of IFN-γ+ spots/10⁶ splenocytes.

### Conclusion

Taken together, the combined T cell responses elicited against the Wuhan RBD (aa319-542) and the additional T cell epitopes encoded in the antigenic unit indicate that combining T cell epitopes with a B cell antigen can broaden the T cell response induced by the vaccine.

### Example 9: Assessment of Humoral Immune Response Induced in Mice Against SARS-COV-2 RBD (Amino Acids 319-542, Wuhan Variant)

Sera from the mice vaccinated with TECH004-IV002, TECH004-IV003, TECH004-IV004, and TECH004-IV005 or the VB1026 control plasmid were collected 12 days after vaccination and tested for anti-RBD IgG antibodies binding the RBD (Wuhan variant) protein.

Briefly, blood was collected from the saphenous vein of the vaccinated mice. Coagulated blood was centrifuged twice (1000 g, 15 min) and the serum was collected and transferred to a clean tube. The humoral immune response was evaluated in an ELISA assay detecting total IgG in the sera binding to RBD (aa319-542) from SARS-COV2 (Wuhan variant). ELISA plates (MaxiSorp Nunc-Immuno plates) were coated with 1 μg/ml recombinant RBD-His protein antigen in PBS overnight at 4° C. Plates were blocked with 4% BSA in PBS for 1 hour at room temperature. Plates were then incubated with serial dilutions of mouse sera and incubated for 2 h at 37° C. Plates were washed 3 times, incubated with 1:50 000 dilution of anti-mouse total IgG-HRP antibody (Southern Biotech) and incubated for 1 h at 37° C. After final washing, plates were developed using TMB substrate (Merck, cat. CL07-1000). Plates were read at 450 nm wavelength within 30 min using a SPARK® Multimode Microplate Reader (Tecan). Binding antibody endpoint titers were calculated as the reciprocal of the highest dilution resulting in a signal above the cutoff. Binding antigens tested included SARS-COV-2 antigens: RBD (Sino Biological 40592-V08H; (SEQ ID NO: 26)).

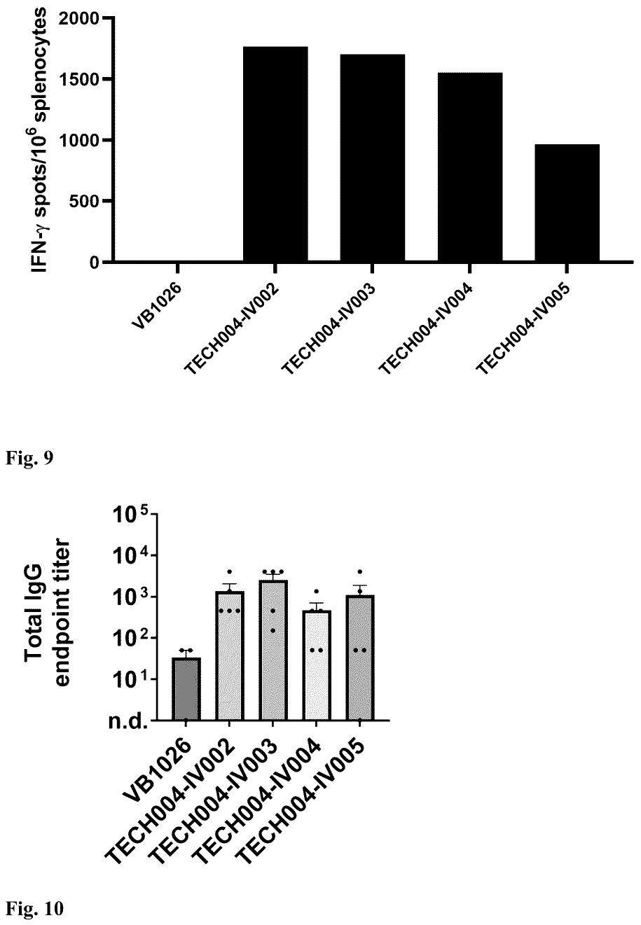

The results shown in FIG. 10 demonstrate that all Combo constructs tested TECH004-IV002, TECH004-IV003, TECH004-IV004, and TECH004-IV005, encoding RBD (amino acids 319-542) from SARS-COV-2 (Wuhan variant) as part of the antigenic unit induced total IgG responses against said antigenic unit.

### Conclusion

These results indicate that irrespective of the varying length of the subunit comprising T cell epitopes, a construct combining such subunits with the RBD (amino acids 319-542) antigen in the antigenic unit elicits antibody responses against the RBD antigen, i.e. the number of T cell epitopes of the subunit does not influence said response.

### Example 10: Design and Production of Influenza A Virus (A/PR/8/34(H1N1)) Combo DNA Constructs

All gene sequences of tested constructs were ordered from Genscript (Genscript Biotech B.V., Netherlands) and cloned into the expression vector pUMVC4a, a master plasmid comprising a nucleotide sequence encoding the signal peptide, targeting unit, dimerization unit and unit linker described in Table 1.

DNA plasmids, TECH004-IV025 (SEQ ID NO: 173), TECH004-V026 (SEQ ID NO: 174), and TECH004-IV027 (SEQ ID NO: 175) comprise nucleic acid sequences encoding elements/units including a targeting unit, a dimerization unit and a unit linker (Table 6) followed by an antigenic unit comprising one or more T cell epitopes (Table 7), an antigen linker and a portion of Hemagglutinin (HA) corresponding to amino acids 18-541 from Influenza A virus (A/Puerto Rico/8/1934 (H1N1; SEQ ID NO: 140)).

TABLE 6

| Elements/Units | Description and sequence |
|---|---|
| Signal peptide | Native leader sequence for human CCL3L1 |
| | Amino acids 1-23 of SEQ ID NO: 1 |
| Targeting unit | Full length human CCL3L1 sequence |
| | Amino acids 24-93 of SEQ ID NO: 1 |
| Dimerization | Human hinge-region 1 from IgG3 |
| unit | Amino acids 94-105 of SEQ ID NO: 1 |
| | Human hinge-region 4 from IgG3 |
| | Amino acids 106-120 of SEQ ID NO: 1 |
| | Glycine-serine-linker |
| | Amino acids 121-130 of SEQ ID NO: 1 |
| | Human CH3 domain from IgG3 |
| | Amino acids 131-237 of SEQ ID NO: 1 |
| Unit linker | Glycine-leucine linker |
| | Amino acids 238-242 of SEQ ID NO: 1 |

TABLE 7

| T cell | | SEQ |
|---|---|---|
| epitope | Sequence | ID NO: |
| pep01 | KLSTRGVQIASNENMETMESSTLELRS | 159 |
| pep02 | GENGRKTRIAYERMCNILKGKFQTAAQ | 160 |
| pep03 | GKMIGGIGRFYIQMCTELKLSDYEGRL | 161 |
| pep04 | LLEMCHSTQIGGIRMVDILRQNPTEEQ | 162 |
| pep05 | IWHSNLNDATYQRTRALVRTGMDPRMC | 163 |
| pep06 | DQSLPPNFSSLENFRAYVDGFEPNGYI | 164 |
| pep07 | MSLLTEVETPIRNEWGCRCNGSSDPLA | 165 |
| pep08 | YEGRLIQNSLTIERMVLSAFDERRNKY | 166 |
| pep09 | NSSLCPIRGWAIYSKDNSIRIGSKGDV | 167 |
| pep10 | RNFWRGENGRKTRIAYERMCNILKGKF | 168 |

TECH004-IV025 (encoding amino acid sequence SEQ ID NO: 173), comprising a subunit with 3 predicted T cell epitopes, pep01 (SEQ ID NO: 159), pep06 (SEQ ID NO: 164) and pep07 (SEQ ID NO: 165), separated by (GGGGS)2 T cell epitope linkers (SEQ ID NO: 24, wherein m=2) and a GGSAGGSGSGSSGGSSGASGTGTAGGTGSGSGTGSG antigen linker (SEQ ID NO: 80), which connects the subunit to the HA antigen (SEQ ID NO: 140).

TECH004-IV026 (encoding amino acid sequence SEQ ID NO: 174), comprising a subunit with 5 predicted T cell epitopes, pep01 (SEQ ID NO: 159), pep02 (SEQ ID NO: 160), pep05 (SEQ ID NO: 163), pep06 (SEQ ID NO: 164) and pep07 (SEQ ID NO: 165), separated by (GGGGS)2 T cell epitope linkers (SEQ ID NO: 24, wherein m=2) and a GGSAGGSGSGSSGGSSGASGTGTAGGTGSGSGTGSG antigen linker (SEQ ID NO: 80), which connects the subunit to the HA antigen (SEQ ID NO: 140).

TECH004-IV027 (encoding amino acid sequence SEQ ID NO: 175), comprising a subunit with 10 predicted T cell epitopes, pep01 (SEQ ID NO: 159), pep02 (SEQ ID NO: 160), pep03 (SEQ ID NO: 161), pep04 (SEQ ID NO: 162), pep05 (SEQ ID NO: 163), pep06 (SEQ ID NO: 164), pep07 (SEQ ID NO: 165), pep08 (SEQ ID NO: 166), pep09 (SEQ ID NO: 167) and pep10 (SEQ ID NO: 168), separated by (GGGGS)2 T cell epitope linkers (SEQ ID NO: 24, wherein m=2) and a GGSAGGSGSGSSGGSSGASGTGTAGGTGSGSGTGSG antigen linker (SEQ ID NO: 80), which connects the subunit to the HA antigen (SEQ ID NO: 140).

### Example 11: Assessment of Expression and Secretion of Proteins Encoded by TECH004-IV025, TECH004-IV026, and TECH004-IV027

The purpose of this study was to characterize protein expression and secretion post transient transfection of mammalian cells with the TECH004-IV025, TECH004-IV026, and TECH004-IV027 plasmids by measuring the presence of secreted proteins in the cell supernatant by an ELISA assay using antibodies against the targeting and dimerization units. Briefly, Expi293F cells (2×10⁶ cells/ml, 1 ml) were seeded in a 96-well culture plate. The cells were transfected with 0.64 μg/ml plasmid DNA using ExpiFectamine 293 Reagent (100014994 Thermo Fisher Sci.), and the plates were incubated on an orbital shaker (3 mm diameter, 900 rpm) in a humidified CO2 cell incubator (8% CO2, 37° C.). The plates were incubated for 72 h before the supernatant was harvested. The secreted proteins were characterized in a sandwich ELISA of the supernatant using mouse anti-human IgG CH3 domain antibody (capture antibody, 100 μl/well, 1 μg/ml, MCA878G, Bio-Rad), rabbit anti-Influenza A H1N1 HA domain antibody (detection antibody, 100 μl/well, 0.2 μg/ml, 11684-R107, Sino Biological), and goat anti-rabbit IgG antibody (secondary antibody, 100 μl/well, 0.16 μg/ml, 31460, Thermo Fisher Scientific).

### Conclusion

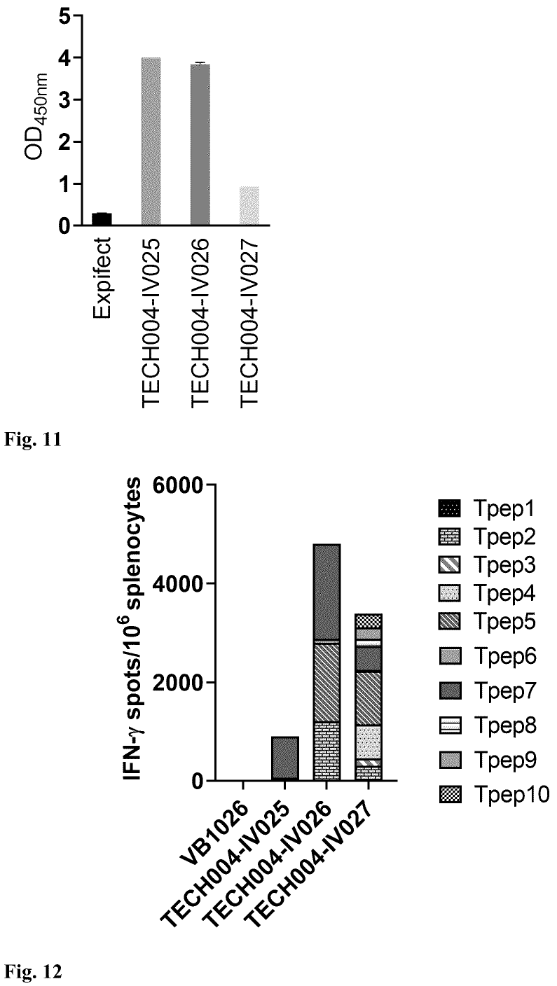

The results presented in FIG. 11 demonstrate successful protein expression and secretion from transfected Expi293F cells of all H1N1 Combo constructs.

### Example 12: Assessment of T Cell Responses Induced Against H1N1 T Cell Epitopes and HA Antigen Encoded in TECH004-IV025, TECH004-IV026, and TECH004-IV027

Female, 6-week-old BALB/c mice were obtained from Janvier Labs (France). All animals were housed in the animal facility at the University of Oslo (Oslo, Norway). All animal protocols were approved by the Norwegian Food Safety Authority (Oslo, Norway). 5 mice/group were used for the testing of the constructs comprising an antigenic unit, whereas 3 mice/group were used for the negative control.

Immunogenicity of TECH004-IV025, TECH004-IV026 and TECH004-IV027 were determined and VB1026 was used as negative control. This DNA plasmid is identical to TECH004-IV025, TECH004-IV026 and TECH004-IV027, but comprises no antigenic unit.

A final dose of 25 μg DNA plasmid dissolved in sterile PBS was administered by intramuscular needle injection to each tibialis anterior (2×25 μl, 500 μg/ml), followed by electroporation with AgilePulse in vivo electroporation system (BTX, USA).

The spleens were collected 14 days after vaccination and mashed in a cell strainer to obtain a single cell suspension. The red blood cells were lysed using ammonium-chloride-potassium (ACK) lysing buffer. The splenocytes were pooled with in respective vaccinated groups and counted using the NucleoCounter NC-202 (ChemoMetec, Denmark) and resuspended to a final concentration of 6×10⁶ cells/ml. The splenocytes were then seeded 6×10⁵ cells/well and re-stimulating with 4 μg/ml of single peptides corresponding to T cell epitopes (Table 7) and 4 μg/ml of single peptides corresponding to three T cell epitopes within HA (Table 8) for 24 hours. No-peptide-stimulation was used as negative control. The stimulated splenocytes were analyzed for IFN-γ responses using the IFN-γ FluoroSpot kit (Mabtech AB, Sweden). Spot-forming cells were measured in a IRIS Fluorospot and ELISpot plate reader (Mabtech AB) and analyzed using the Apex software (Mabtech AB). Results are shown as the mean number of IFN-γ+ spots/10⁶ splenocytes.

TABLE 8

| T cell epitope | Peptide composition |
|---|---|
| Pep11 | SVSSFERFEIFPK (SEQ ID NO: 176) |
| Pep12 | HNTNGVTAACSHEG (SEQ ID NO: 177) |
| Pep13 | IYSTVASSL (SEQ ID NO: 178) |

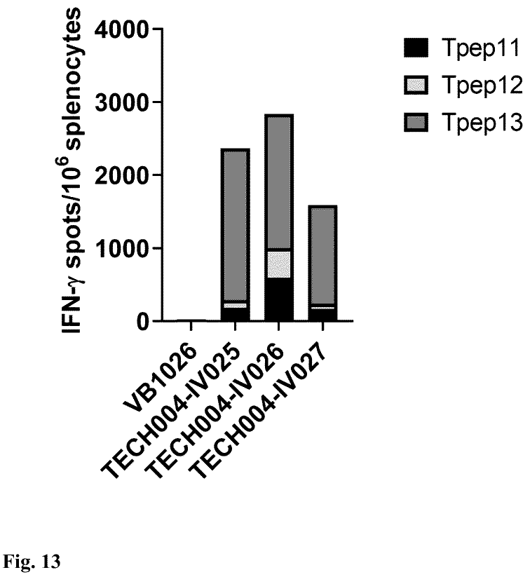

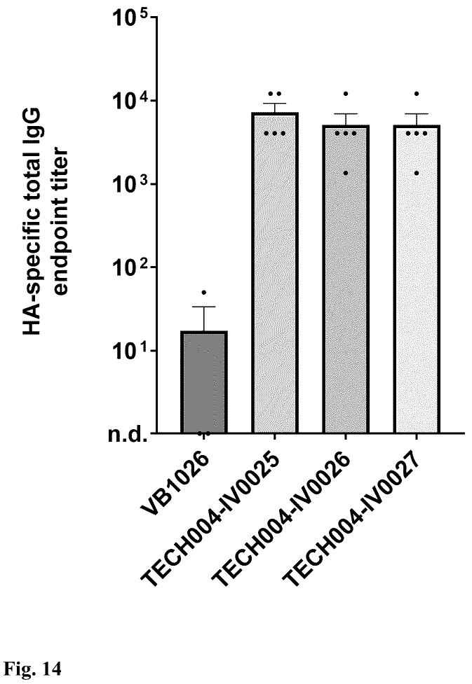

T cell responses against the predicted T cell epitopes from H1N1 strain were detected in spleens at day 14 from mice vaccinated once with 25 μg of plasmid DNA constructs containing either 3, 5 or 10 predicted T cell epitopes (FIG. 12). In addition, all constructs were also able to elicit a HA-specific T cell response (FIG. 13).

### Conclusion

Taken together, the combined T cell responses elicited against the Influenza A virus H1N1 HA antigen and the additional T cell epitopes encoded in the antigenic unit indicate that combining T cell epitopes with a B cell antigen can broaden the T cell response induced by the vaccine.

### Example 13: Assessment of Humoral Immune Response Induced in Mice Against H1N1 HA Antigen

Sera from the mice vaccinated with TECH004-IV025, TECH004-IV026 and TECH004-IV027 or the VB1026 control plasmid were collected 14 days after vaccination and tested for total IgG antibodies binding the HA protein.

Briefly, blood was collected from the saphenous vein of the vaccinated mice. Coagulated blood was centrifuged twice (1000 g, 15 min) and the serum was collected and transferred to a clean tube. The humoral immune response was evaluated in an ELISA assay detecting total IgG in the sera binding to HA (18-541aa) from Influenza A virus (A/Puerto Rico/8/1934(H1N1). ELISA plates (MaxiSorp Nunc-Immuno plates) were coated with 1 μg/ml recombinant HA-His protein antigen in PBS overnight at 4° C. Plates were blocked with 4% BSA in PBS for 1 hour at RT. Plates were then incubated with serial dilutions of mouse sera and incubated for 2 h at 37° C. Plates were washed 3× and incubated with 1:50 000 dilution of anti-mouse total IgG-HRP antibody (Southern Biotech) and incubated for 1 h at 37° C. After final washing, plates were developed using TMB substrate (Merck, cat. CL07-1000). Plates were read at 450 nm wavelength within 30 min using a SPARK® Multimode Microplate Reader (Tecan). Binding antibody endpoint titers were calculated as the reciprocal of the highest dilution resulting in a signal above the cutoff. Binding antigens tested included Influenza A H1N1 (A/Puerto Rico/8/1934) Hemagglutinin/HA Protein (Sino Biological 11684-V08H; (SEQ ID NO: 179)).

### Conclusion

The results shown in FIG. 14 demonstrate that all constructs tested, namely TECH004-IV025, TECH004-IV026, and TECH004-IV027, encoding HA (amino acids 18-541) from Influenza A virus (A/Puerto Rico/8/1934(H1N1) as part of the antigenic unit, induced total IgG responses against HA. This indicates that, irrespective of combining a string of varying lengths of 3-10 T cell epitopes in addition to the HA antigen in the antigenic unit of the construct, the vaccines designed according to the invention induce similar antibody responses against the antigen.

### SEQUENCES

The numbers included in some of the sequences below indicate the position to facilitate the reader's understanding.

SEQ ID NO: 1
Amino acid sequence of human MIP1-a (amino acids 24-93), signal peptide of human
MIP1-α (amino acids 1-23), hinge exon h1 from IgG3 (amino acids 94-105), hinge
exon h4 from IgG3 (amino acids 106-120), a dimerization unit linker (amino acids
121-130) and the human CH3 domain of IgG3 (amino acids 131-237)
M1QVSTAALAVLLCTMALCNQVLS²³A²⁴PLAADTPTACCFSYTSRQIPQNFIAD
YFETSSQCSKPSVIFLTKRGRQVCADPSEEWVQKYVSDLELSA⁹³E⁹⁴LKTPLGDT
THT¹⁰⁵E¹⁰⁶PKSCDTPPPCPRCP¹²⁰G¹²¹GGSSGGGSG¹³⁰G¹³¹QPREPQVYTLPPSREE
MTKNQVSLTCLVKGFYPSDIAVEWESSGQPENNYNTTPPMLDSDGSFFLYSKL
TVDKSRWQQGNIFSCSVMHEALHNRFTQKSLSLSPGK²³⁷
SEQ ID NO: 2
Amino acid sequence of anti-pan HLA class II with Ig VH signal peptide (amino acids
1-19), anti-pan HLA class II VL (amino acids 20-127), a linker (amino acids 128-142)
and anti-pan HLA class II VH (amino acids 143-260)
M1NFGLRLIFLVLTLKGVQC¹⁹D²⁰IQMTQTTSSLSASLGDRVTISCSASQDINNYL
NWYQQKPDGTVKLLIYYTSSLHSGVPSRFSGSGSGTDYSLTISNLEPEDIATYY
CQQYSKFPRTFGGGTKLEIKR¹²⁷G¹²⁸GGGSGGGGSGGGGS¹⁴²Q¹⁴³IQLVQSGPEL
KKPGETVKISCKASGYTFINYGMNWVKQTPGKGLKWMGWINTYSGEPTYPD
DFKGRFAFSLETSASTAYLQLNNLKNEDMATYFCARGDYYGPFDNWGQGTTL
TVSS²⁶⁰
SEQ ID NO: 3
Signal peptide
MDAMKRGLCCVLLLCGAVFVSP
SEQ ID NO: 4
Amino acid sequence of VB2081
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGGGGSGGGGSRVQPTESIVRFPNITNLCPFGEVENATRFASVYAWNRKRISN
CVADYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKPFE
RDISTEIYQAGSTPCNGVEGENCYFPLQSYGFQPTNGVGYQPYRVVVLSFELLH
APATVCGPKKSTNLVKNKCVNF
SEQ ID NO: 5
Amino acid sequence of VB2082
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLFVNEFYAYLRKHFSMMGGGGSGG
GGSRVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTEIYQA
GSTPCNGVEGFNCYFPLQSYGFQPTNGVGYQPYRVVVLSFELLHAPATVCGPK
KSTNLVKNKCVNF
SEQ ID NO: 6
Amino acid sequence of VB2083
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGGGGSGGGGSFVNEFYAYLRKHFSMMGGGGSGGGGSRVQPTESIVRFPNIT
NLCPFGEVENATRFASVYAWNRKRISNCVADYSVLYNSASFSTFKCYGVSPTK
LNDLCFTNVYADSFVIRGDEVRQIAPGQTGKIADYNYKLPDDFTGCVIAWNSN
NLDSKVGGNYNYLYRLFRKSNLKPFERDISTEIYQAGSTPCNGVEGENCYFPL
QSYGFQPTNGVGYQPYRVVVLSFELLHAPATVCGPKKSTNLVKNKCVNF
SEQ ID NO: 7
Amino acid sequence of VB2084
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGGGGSGGGGSFVNEFYAYLRKHFSMMGGGGSGGGGSLVKPSFYVYSRVKN
LGGGGSGGGGSRVQPTESIVRFPNITNLCPFGEVENATRFASVYAWNRKRISNC
VADYSVLYNSASFSTFKCYGVSPTKLNDLCFTNVYADSFVIRGDEVRQIAPGQ
TGKIADYNYKLPDDFTGCVIAWNSNNLDSKVGGNYNYLYRLFRKSNLKPFER
DISTEIYQAGSTPCNGVEGENCYFPLQSYGFQPTNGVGYQPYRVVVLSFELLHA
PATVCGPKKSTNLVKNKCVNF
SEQ ID NO: 8
Amino acid sequence of VB2085
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGLGGLRVQPTESIVRFPNITNLCPFGEVENATRFASVYAWNRKRISNCVADYS
VLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIY
QAGSTPCNGVEGENCYFPLQSYGFQPTNGVGYQPYRVVVLSFELLHAPATVC
GPKKSTNLVKNKCVNF
SEQ ID NO: 9
Amino acid sequence of VB2086
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGLGGLGLGGLRVQPTESIVRFPNITNLCPFGEVFNATRFASVYAWNRKRISNC
VADYSVLYNSASFSTFKCYGVSPTKLNDLCFTNVYADSFVIRGDEVRQIAPGQ
TGKIADYNYKLPDDFTGCVIAWNSNNLDSKVGGNYNYLYRLFRKSNLKPFER
DISTEIYQAGSTPCNGVEGENCYFPLQSYGFQPTNGVGYQPYRVVVLSFELLHA
PATVCGPKKSTNLVKNKCVNF
SEQ ID NO: 10
Amino acid sequence of VB2087
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLFVNEFYAYLRKHFSMMGLGGLRV
QPTESIVRFPNITNLCPFGEVENATRFASVYAWNRKRISNCVADYSVLYNSASF
STFKCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTN
LVKNKCVNF
SEQ ID NO: 11
Amino acid sequence of VB2088
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGGGGSGGGGSFVNEFYAYLRKHFSMMGLGGLRVQPTESIVRFPNITNLCPFG
EVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKV
GGNYNYLYRLFRKSNLKPFERDISTEIYQAGSTPCNGVEGENCYFPLQSYGFQP
TNGVGYQPYRVVVLSFELLHAPATVCGPKKSTNLVKNKCVNF
SEQ ID NO: 12
Amino acid sequence of VB2089
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGGGGSGGGGSFVNEFYAYLRKHFSMMGGGGSGGGGSLVKPSFYVYSRVKN
LGLGGLRVQPTESIVRFPNITNLCPFGEVENATRFASVYAWNRKRISNCVADYS
VLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIY
QAGSTPCNGVEGFNCYFPLQSYGFQPTNGVGYQPYRVVVLSFELLHAPATVC
GPKKSTNLVKNKCVNF
SEQ ID NO: 13
Amino acid sequence of VB2091
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VTQKSLSLSPGKGLGGLRVQPTESIVRFPNITNLCPFGEVENATRFASVYAWNR
KRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLS
FELLHAPATVCGPKKSTNLVKNKCVNF
SEQ ID NO: 14
Amino acid sequence of VB2092
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGGGGSGGGGSFVNEFYAYLRKHFSMMGGGGSGGGGSLVKPSFYVYSRVKN
LTQKSLSLSPGKGLGGLRVQPTESIVRFPNITNLCPFGEVFNATRFASVYAWNR
KRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLS
FELLHAPATVCGPKKSTNLVKNKCVNF
SEQ ID NO: 15
Amino acid sequence of VB2094
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VSLSLSPGKGLGGLRVQPTESIVRFPNITNLCPFGEVENATRFASVYAWNRKRI
SNCVADYSVLYNSASFSTFKCYGVSPTKLNDLCFTNVYADSFVIRGDEVRQIA
PGQTGKIADYNYKLPDDFTGCVIAWNSNNLDSKVGGNYNYLYRLFRKSNLKP
FERDISTEIYQAGSTPCNGVEGENCYFPLQSYGFQPTNGVGYQPYRVVVLSFEL
LHAPATVCGPKKSTNLVKNKCVNF
SEQ ID NO: 16
Amino acid sequence of VB2095
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGGGGGGGGSFVNEFYAYLRKHFSMMGGGGSGGGGSLVKPSFYVYSRVKN
LSLSLSPGKGLGGLRVQPTESIVRFPNITNLCPFGEVENATRFASVYAWNRKRIS
NCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPF
ERDISTEIYQAGSTPCNGVEGENCYFPLQSYGFQPTNGVGYQPYRVVVLSFELL
HAPATVCGPKKSTNLVKNKCVNF
SEQ ID NO: 17
Amino acid sequence of VB2097
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGGGGSGGGGSFVNEFYAYLRKHFSMMGGGGSGGGGSLVKPSFYVYSRVKN
LGGSAGGSGSGSSGGSSGASGTGTAGGTGSGSGTGSGRVQPTESIVRFPNITNL
CPFGEVENATRFASVYAWNRKRISNCVADYSVLYNSASFSTFKCYGVSPTKLN
DLCFTNVYADSFVIRGDEVRQIAPGQTGKIADYNYKLPDDFTGCVIAWNSNNL
DSKVGGNYNYLYRLFRKSNLKPFERDISTEIYQAGSTPCNGVEGENCYFPLQSY
GFQPTNGVGYQPYRVVVLSFELLHAPATVCGPKKSTNLVKNKCVNF
SEQ ID NO: 18
Amino acid sequence of VB2099
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLHFAIGLALYYPSARIVYTACSHAA
VGGGGSGGGGSFVNEFYAYLRKHFSMMGGGGSGGGGSLVKPSFYVYSRVKN
LGGSGGGSEGGGSEGGGSEGGGSEGGGSEGGGSGGGSRVQPTESIVRFPNITN
LCPFGEVENATRFASVYAWNRKRISNCVADYSVLYNSASFSTFKCYGVSPTKL
NDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGENCYFPLQS

### EMBODIMENTS

1. An immunogenic construct comprising:
(i) a polynucleotide comprising a nucleotide sequence encoding a polypeptide comprising:
   a) a targeting unit targeting or capable of targeting antigen-presenting cells,
   b) a multimerization unit such as a dimerization unit, and
   c) an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
wherein the antigenic unit comprises a subunit comprising the T cell epitopes which are separated by each other by T cell epitope linkers, if more than one T cell epitope is comprised in the subunit, and wherein the subunit is connected to the dimerization unit by a unit linker and separated from the one or more antigens or parts thereof by an antigen linker.

2. The construct according to embodiment 1, wherein the one or more T cell epitopes are from a pathogen, such a from a virus or a pathogenic bacteria.

3. The construct according to any one of the preceding embodiments, wherein the one or more antigens or parts or fragments thereof are from a pathogen, such as from a virus or a pathogenic bacteria.

4. The construct according to any one of the preceding embodiments, wherein the construct comprises a multimerization unit.

5. The construct according to any one of the preceding embodiments, wherein the construct comprises a dimerization unit.

6. The construct according to any one of the preceding embodiments, wherein the one or more antigen is a full-length protein of a pathogen, preferably a full-length surface protein of a pathogen.

7. The construct according to any one of the preceding embodiments, wherein the one or more antigen is a full-length viral protein, preferably a full-length viral envelope protein.

8. The construct according to any one of embodiments 1 to 6, wherein the one or more antigen is a full-length bacterial protein, preferably a full-length bacterial protein that is secreted or surface-localized.

9. The construct according to any one of the preceding embodiments, wherein the antigenic unit comprises one full-length protein of a pathogen, preferably one full-length surface protein of a pathogen.

10. The construct according to any one of the preceding embodiments, wherein the antigenic unit comprises several full-length proteins of a pathogen, preferably several full-length surface proteins of a pathogen.

11. The construct according to any one of the preceding embodiments, wherein the antigenic unit comprises one or more parts or fragments of one or more antigens.

12. The construct according to embodiment 11, wherein the antigenic unit comprises one part or fragment of one antigen.

13. The construct according to embodiment 11, wherein the antigenic unit comprises several parts or fragments of one antigen.

14. The construct according to embodiment 11, wherein the antigenic unit comprises one part or fragment of several antigens.

15. The construct according to embodiment 11, wherein the antigenic unit comprises several parts or fragments of several antigens.

16. The construct according to any one of the preceding embodiments, wherein the antigen is a surface protein of a pathogen.

17. The construct according to embodiment 16, wherein the antigen is a viral surface protein, preferably a viral envelope protein.

18. The construct according to any one of the preceding embodiments, wherein the antigen is a bacterial protein, preferably a bacterial protein that is secreted or surface-localized.

19. The construct according to any one of the preceding embodiments, wherein the antigenic unit comprises multiple copies of antigens or parts or fragments thereof.

20. The construct according to embodiment 19, wherein the antigenic unit comprises from 2 to 5 copies of antigens or parts or fragments thereof.

21. The construct according to any one of the preceding embodiments, wherein the antigen or parts or fragments thereof comprises at least one B cell epitope, preferably several B cell epitopes.

22. The construct according to any one of the preceding embodiments, wherein the subunit comprises n−1 T cell epitope linkers, wherein n is the number of neoepitopes in said antigenic unit and n is an integer of from 1 to 50.

23. The construct according to any one of the preceding embodiments, wherein the subunit comprises 1 to 50 T cell epitopes.

24. The construct according to embodiment 22, wherein the subunit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes or comprises 1 to 3 T cell epitopes or 1 to 5 T cell epitopes or 3 to 6 T cell epitopes or 5 to 15 T cell epitopes or 7 to 17 T cell epitopes or 9 to 19 T cell epitopes.

25. The construct according to any one of the preceding embodiments, wherein the subunit comprises one T cell epitope.

26. The construct according to any one of the preceding embodiments, wherein the subunit comprises several T cell epitopes.

27. The construct according to any one of the preceding embodiments, wherein the T cell epitope is known to be immunogenic or is selected based on its predicted ability to bind to HLA class I/II alleles.

28. The construct according to one of the preceding embodiments, wherein one or more of the T cell epitopes are known to be immunogenic and/or one or more of the T cell epitopes are selected based on the predicted ability to bind to HLA class I/II alleles.

29. The construct according to any one of the preceding embodiments, wherein the T cell epitope is comprised in a structural protein or in a non-structural protein.

30. The construct according to any one of the preceding embodiments, wherein the T cell epitope is from a conserved region of the pathogen.

31. The construct according to any one of the preceding embodiments, wherein the T cell epitope has a length of from 7 to about 200 amino acids, such as from 7 to 100 amino acids, such as from 7 to 150 amino acids, preferably of from 7 to 100 amino acids, e.g. from about 10 to about 100 amino acids or from about 15 to about 100 amino acids or from about 20 to about 75 amino acids or from about 25 to about 50 amino acids; or the T cell epitope has a length suitable for presentation by HLA class I/II alleles, preferably a length of from 7 to 30 amino acids, more preferably a length of from 8 to 15 amino acids.

32. The construct according to any one of the preceding embodiments, wherein the antigenic unit comprises up to 3500 amino acids, such as from 60 to 3500 amino acids, e.g. from about 80 or about 100 or about 150 amino acids to about a 3000 amino acids, such as from about 200 to about 2500 amino acids, such as from about 300 to about 2000 amino acids or from about 400 to about 1500 amino acids or from about 500 to about 1000 amino acids.

33. The construct according to any one of the preceding embodiments, wherein the subunit comprises more than one T cell epitope, and the T cell epitope linker is a peptide consisting of from 4 to 40 amino acids, such as from 4 to 20 amino acids, such as from 5 to 20 amino acids or 5 to 15 amino acids or 8 to 20 amino acids or 8 to 15 amino acids 10 to 15 amino acids or 8 to 12 amino acids; preferably the T cell epitope linker is a peptide consisting of 10 amino acids.

34. The construct according to any one of the preceding embodiments, wherein the antigen linker is a peptide consisting of from 10 to 80 amino acids, e.g. from 11 to 70 amino acids or 15 to 60 amino acids or 20 to 50 amino acids or 25 to 45 amino acids or 12 to 45 amino acids or 13 to 40 amino acids or 30 to 40 amino acids.

35. The construct according to any one of the preceding embodiments, wherein the antigenic unit comprises several antigens or several parts of one or more antigens, and said several antigens or several parts of one or more antigens are separated from each other by an antigen linker as defined in embodiment 34.

36. The construct according to any one of the preceding embodiments, wherein the T cell epitope linker and the antigen linker are more non-immunogenic and/or flexible, preferably non-immunogenic and flexible.

37. The construct according to any one of the preceding embodiments, wherein the T cell epitope linker, if present, and the antigen linker are selected from the group consisting of: serine and/or glycine rich linkers which optionally comprise at least one leucine residue; GSAT linkers; and SEG linkers.

38. The construct according to any one of the preceding embodiments, wherein the T cell epitope linker, if present, and the antigen linker are selected from the group consisting of serine and/or glycine rich linkers which optionally comprise at least one leucine residue, TQKSLSLSPGKGLGGL (SEQ ID NO: 78), SLSLSPGKGLGGL (SEQ ID NO: 79), GSAT linkers such as GGSAGGSGSGSSGGSSGASGTGTAGGTGSGSGTGSG (SEQ ID NO: 80) and SEG linkers such as GGSGGGSEGGGSEGGGSEGGGSEGGGSEGGGSGGGS (SEQ ID NO: 81).

39. The construct according to any one of the preceding embodiments, wherein said targeting unit comprises antibody binding regions with specificity for surface molecules or receptors on antigen presenting cells (APCs), preferably wherein the surface molecule or receptor on APC is selected from the group consisting of HLA, CD14, CD40, CLEC9A, chemokine receptors, such as CCR1, CCR3, CCR4, CCR5, CCR6, CCR7, CCR8 or XCR1, and Toll-like receptors such as TLR-2, TLR-4 or TLR-5.

40. The construct according to any one of the preceding embodiments, wherein the targeting unit has affinity for a chemokine receptor selected from CCR1, CCR3 and CCR5.

41. The construct according to any one of the preceding embodiments, wherein the targeting unit has affinity for MHC class II proteins, preferably MHC class II proteins selected from the group consisting of anti-HLA-DP, anti-HLA-DR and anti-pan HLA class II.

42. The construct according to any one of the preceding embodiments, wherein the targeting unit comprises or consists of pan-HLAII or MIP-1α, preferably pan-HLAII or human MIP-1α (LD78β, CCL3L1).

43. The construct according to embodiment 42, wherein the targeting unit is MIP-1α, preferably human MIP-1α.

44. The construct according to embodiment 43, wherein the targeting unit comprises or consists of an amino acid sequence having at least 80% sequence identity to the amino acid sequence 24-93 of SEQ ID NO: 1, such as at least 85% or at least 86%, 87%, 88%, 89%, 90%, 91%, 92%, 93%, 94%, 95%, 96%, 97%, 98%, at least 99% or 100% sequence identity.

45. The construct according to any one of embodiments 1 to 42, wherein the targeting unit is anti-pan-HLA class II.

46. The construct according to embodiment 45, wherein the targeting unit comprises an amino acid sequence having at least 80% sequence identity to the amino acid sequence 20-260 of SEQ ID NO: 2, such as at least 85% or at least 86%, 87%, 88%, 89%, 90%, 91%, 92%, 93%, 94%, 95%, 96%, 97%, 98%, at least 99% or 100% sequence identity.

47. The construct according to any of the preceding embodiments, wherein the multimerization unit is a dimerization unit which comprises a hinge region, such as hinge exon h1 and hinge exon h4.

48. The construct according to embodiment 47, wherein the hinge region has the ability to form one or more covalent bonds.

49. The construct according to any one of embodiments 47 to 48, wherein the hinge region is Ig derived.

50. The construct according to any one of embodiments 47 to 49, wherein the dimerization unit further comprises another domain that facilitates dimerization.

51. The construct according to embodiment 50, wherein the other domain is an immunoglobulin domain, preferably an immunoglobulin constant domain.

52. The construct according to any one of embodiments 50 to 51, wherein the other domain is a carboxyterminal C domain derived from IgG, preferably from IgG3.

53. The construct according to any one of embodiments 47 to 52, wherein the dimerization unit further comprises a dimerization unit linker, such as glycine-serine rich linker, such as GGGSSGGGSG (SEQ ID NO: 87).

54. The construct according to embodiment 53, wherein the dimerization unit linker connects the hinge region and the other domain that facilitates dimerization.

55. The construct according to any of embodiments 47 to 54, wherein the dimerization unit comprises hinge exon h1 and hinge exon h4, a dimerization unit linker and a CH3 domain of human IgG3.

56. The construct according to embodiment 55, wherein the dimerization unit comprises or consists of an amino acid sequence having at least 80% sequence identity to the amino acid sequence 94 to 237 of SEQ ID NO: 1, such as at least 85%, or at least 86%, 87%, 88%, 89%, 90%, 91%, 92%, 93%, 94%, 95%, 96%, 97%, 98%, at least 99% or 100% sequence identity.

57. The construct according to any one of the preceding embodiments, wherein the construct is the polynucleotide (i).

58. The construct according to embodiment 57, wherein the polynucleotide is an RNA or DNA, preferably a DNA.

59. The construct according to any one of embodiments 1 to 58, wherein the polynucleotide further comprises a nucleotide sequence encoding a signal peptide.

60. The construct according to embodiment 59, wherein the signal peptide is selected from the list consisting of Ig VH signal peptide, human TPA signal peptide and human MIP-1α signal peptide.

61. The construct according to embodiment 60, wherein the signal peptide comprises or consists of an amino acid sequence having at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the amino acid sequence 1-23 of SEQ ID NO: 1.

62. The construct according to embodiment 61, wherein the targeting unit is human MIP-1α.

63. The construct according to embodiment 60, wherein the signal peptide comprises or consists of an amino acid sequence having at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the amino acid sequence 1-19 of SEQ ID NO: 2.

64. The construct according to embodiment 63, wherein the targeting unit is anti-pan HLA class II.

65. A polynucleotide as defined in any one of the preceding embodiments.

66. A polynucleotide comprising a nucleotide sequence encoding a polypeptide comprising:
a) a targeting unit targeting or capable of targeting antigen-presenting cells,
b) a multimerization unit such as a dimerization unit, and
c) an antigenic unit comprising one or more T cell epitopes and one or more antigens or parts or fragments thereof,
   wherein the antigenic unit comprises a subunit comprising the T cell epitopes which are separated by each other by T cell epitope linkers, if more than one T cell epitope is comprised in the subunit,
   and wherein the subunit is connected to the dimerization unit by a unit linker and separated from the one or more antigens or parts thereof by an antigen linker.

67. The polynucleotide according to any one of embodiments 65 to 66, wherein the one or more T cell epitopes are from a pathogen, such a from a virus or a pathogenic bacteria.

68. The polynucleotide according to any one of embodiments 65 to 67, wherein the one or more antigens are from a pathogen, such a from a virus or a pathogenic bacteria.

69. The polynucleotide according to any one of embodiments 65 to 67, wherein the construct comprises a dimerization unit.

70. The polynucleotide according to any one of embodiments 65 to 69, wherein the construct comprises a multimerization unit.

71. The polynucleotide according to any one of embodiments 65 to 70, wherein the one or more antigen is a full-length protein of a pathogen, preferably a full-length surface protein of a pathogen.

72. The polynucleotide according to any one of embodiments 65 to 71, wherein the one or more antigen is a full-length viral protein, preferably a full-length viral envelope protein.

73. The polynucleotide according to any one of embodiments 65 to 71, wherein the one or more antigen is a full-length bacterial protein, preferably a full-length bacterial protein that is secreted or surface-localized.

74. The polynucleotide according to any one of embodiments 65 to 73, wherein the antigenic unit comprises one full-length protein of a pathogen, preferably one full-length surface protein of a pathogen.

75. The polynucleotide according to any one of embodiments 65 to 74, wherein the antigenic unit comprises several full-length proteins of a pathogen, preferably several full-length surface proteins of a pathogen.

76. The polynucleotide according to any one of embodiments 65 to 75, wherein the antigenic unit comprises one or more parts or fragments of one or more antigens.

77. The polynucleotide according to embodiment 76, wherein the antigenic unit comprises one part or fragment of one antigen.

78. The polynucleotide according to embodiment 76, wherein the antigenic unit comprises several parts or fragments of one antigen.

79. The polynucleotide according to embodiment 76, wherein the antigenic unit comprises one part or fragment of several antigens.

80. The polynucleotide according to embodiment 76, wherein the antigenic unit comprises several parts or fragments of several antigens.

81. The polynucleotide according to any one embodiments 65 to 80, wherein the antigen is a surface protein of a pathogen.

82. The polynucleotide according to embodiment 81, wherein the antigen is a viral surface protein, preferably a viral envelope protein.

83. The polynucleotide according to any one of the preceding embodiments, wherein the antigen is a bacterial protein, preferably a bacterial protein that is secreted or surface-localized.

84. The construct according to any one of embodiments 65 to 83, wherein the antigenic unit comprises multiple copies of antigens or parts or fragments thereof.

85. The polynucleotide according to embodiment 84, wherein the antigenic unit comprises from 2 to 5 copies of antigens or parts or fragments thereof.

86. The polynucleotide according to any one of embodiments 65 to 85, wherein the antigen or parts or fragments thereof comprises at least one B cell epitope, preferably several B cell epitopes.

87. The polynucleotide according to any one of embodiments 65 to 86, wherein the subunit comprises n−1 T cell epitope linkers, wherein n is the number of neoepitopes in said antigenic unit and n is an integer of from 1 to 50.

88. The polynucleotide according to any one of embodiments 65 to 87, wherein the subunit comprises 1 to 50 T cell epitopes.

89. The construct according to embodiment 88, wherein the subunit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes or comprises 1 to 3 T cell epitopes or 1 to 5 T cell epitopes or 3 to 6 T cell epitopes or 5 to 15 T cell epitopes or 7 to 17 T cell epitopes or 9 to 19 T cell epitopes.

90. The polynucleotide according to any one of embodiments 65 to 89, wherein the subunit comprises one T cell epitope.

91. The polynucleotide according to any one of embodiments 65 to 90, wherein the subunit comprises several T cell epitopes.

92. The polynucleotide according to any one of embodiments 65 to 91, wherein the T cell epitope is known to be immunogenic or is selected based on its predicted ability to bind to HLA class I/II alleles.

93. The polynucleotide according to any one of embodiments 65 to 92, wherein one or more of the T cell epitopes are known to be immunogenic and/or one or more of the T cell epitopes are selected based on the predicted ability to bind to HLA class I/II alleles.

94. The polynucleotide according to any one of embodiments 65 to 93, wherein the T cell epitope is comprised in a structural protein or in a non-structural protein.

95. The polynucleotide according to any one of embodiments 65 to 94, wherein the T cell epitope is from a conserved region of the pathogen.

96. The polynucleotide according to any one of embodiments 65 to 95, wherein the T cell epitope has a length of from 7 to about 200 amino acids, such as from 7 to 100 amino acids, such as from 7 to 150 amino acids, preferably of from 7 to 100 amino acids, e.g. from about 10 to about 100 amino acids or from about 15 to about 100 amino acids or from about 20 to about 75 amino acids or from about 25 to about 50 amino acids; or the T cell epitope has a length suitable for presentation by HLA class I/II alleles, preferably a length of from 7 to 30 amino acids, more preferably a length of from 8 to 15 amino acids.

97. The polynucleotide according to any one of embodiments 65 to 96, wherein the antigenic unit comprises up to 3500 amino acids, such as from 60 to 3500 amino acids, e.g. from about 80 or about 100 or about 150 amino acids to about a 3000 amino acids, such as from about 200 to about 2500 amino acids, such as from about 300 to about 2000 amino acids or from about 400 to about 1500 amino acids or from about 500 to about 1000 amino acids.

98. The polynucleotide according to any one of embodiments 65 to 97, wherein the subunit comprises more than one T cell epitope, and the T cell epitope linker is a peptide consisting of from 4 to 40 amino acids, such as from 4 to 20 amino acids, such as from 5 to 20 amino acids or 5 to 15 amino acids or 8 to 20 amino acids or 8 to 15 amino acids 10 to 15 amino acids or 8 to 12 amino acids; preferably the T cell epitope linker is a peptide consisting of 10 amino acids.

99. The polynucleotide according to any one of embodiments 65 to 98, wherein the antigen linker is a peptide consisting of from 10 to 80 amino acids, e.g. from 11 to 70 amino acids or 15 to 60 amino acids or 20 to 50 amino acids or 25 to 45 amino acids or 12 to 45 amino acids or 13 to 40 amino acids or 30 to 40 amino acids.

100. The polynucleotide according to any one of embodiments 65 to 99, wherein the antigenic unit comprises several antigens or several parts of one or more antigens, and said several antigens or several parts of one or more antigens are separated from each other by an antigen linker as defined in embodiment 99.

101. The polynucleotide according to any one of embodiments 65 to 100, wherein the T cell epitope linker and the antigen linker are more non-immunogenic and/or flexible, preferably non-immunogenic and flexible.

102. The polynucleotide according to any one of embodiments 65 to 101, wherein the T cell epitope linker, if present, and the antigen linker are selected from the group consisting of: serine and/or glycine rich linkers which optionally comprise at least one leucine residue; GSAT linkers; and SEG linkers.

103. The polynucleotide according to any one of embodiments 65 to 102, wherein the T cell epitope linker, if present, and the antigen linker are selected from the group consisting of serine and/or glycine rich linkers which optionally comprise at least one leucine residue, TQKSLSLSPGKGLGGL (SEQ ID NO: 78), SLSLSPGKGLGGL (SEQ ID NO: 79), GSAT linkers such as GGSAGGSGSGSSGGSSGASGTGTAGGTGSGSGTGSG (SEQ ID NO: 80) and SEG linkers such as GGSGGGSEGGGSEGGGSEGGGSEGGGSEGGGSGGGS (SEQ ID NO: 81).

104. The polynucleotide according to any one of embodiments 65 to 103, wherein said targeting unit comprises antibody binding regions with specificity for surface molecules or receptors on antigen presenting cells (APCs), preferably wherein the surface molecule or receptor on APC is selected from the group consisting of HLA, CD14, CD40, CLEC9A, chemokine receptors, such as CCR1, CCR3, CCR4, CCR5, CCR6, CCR7, CCR8 or XCR1, and Toll-like receptors such as TLR-2, TLR-4 or TLR-5.

105. The polynucleotide according to any one of embodiments 65 to 104, wherein the targeting unit has affinity for a chemokine receptor selected from CCR1, CCR3 and CCR5.

106. The polynucleotide according to any one of embodiments 65 to 105, wherein the targeting unit has affinity for MHC class II proteins, preferably MHC class II proteins selected from the group consisting of anti-HLA-DP, anti-HLA-DR and anti-pan HLA class II.

107. The polynucleotide according to any one of embodiments 65 to 106, wherein the targeting unit comprises or consists of pan-HLAII or MIP-1α, preferably pan-HLAII or human MIP-1α (LD78β, CCL3L1).

108. The polynucleotide according to embodiment 107, wherein the targeting unit is MIP-1α, preferably human MIP-1α.

109. The polynucleotide according to embodiment 108, wherein the targeting unit comprises or consists of an amino acid sequence having at least 80% sequence identity to the amino acid sequence 24-93 of SEQ ID NO: 1, such as at least 85% or at least 86%, 87%, 88%, 89%, 90%, 91%, 92%, 93%, 94%, 95%, 96%, 97%, 98%, at least 99% or 100% sequence identity.

110. The polynucleotide according to any one of embodiments 65 to 109, wherein the targeting unit is anti-pan-HLA class II.

111. The polynucleotide according to embodiment 110, wherein the targeting unit comprises an amino acid sequence having at least 80% sequence identity to the amino acid sequence 20-260 of SEQ ID NO: 2, such as at least 85% or at least 86%, 87%, 88%, 89%, 90%, 91%, 92%, 93%, 94%, 95%, 96%, 97%, 98%, at least 99% or 100% sequence identity.

112. The polynucleotide according to any one of embodiments 65 to 111, wherein the multimerization unit is a dimerization unit which comprises a hinge region, such as hinge exon h1 and hinge exon h4.

113. The polynucleotide according to embodiment 112, wherein the hinge region has the ability to form one or more covalent bonds.

114. The polynucleotide according to any one of embodiments 65 to 113, wherein the hinge region is Ig derived.

115. The polynucleotide according to any one of embodiments 65 to 114, wherein the dimerization unit further comprises another domain that facilitates dimerization.

116. The polynucleotide according to embodiment 115, wherein the other domain is an immunoglobulin domain, preferably an immunoglobulin constant domain.

117. The polynucleotide according to any one of embodiments 115 to 116, wherein the other domain is a carboxyterminal C domain derived from IgG, preferably from IgG3.

118. The polynucleotide according to any one of embodiments 65 to 117, wherein the dimerization unit further comprises a dimerization unit linker, such as 30 glycine-serine rich linker, such as GGGSSGGGSG (SEQ ID NO: 87).

119. The polynucleotide according to embodiment 118, wherein the dimerization unit linker connects the hinge region and the other domain that facilitates dimerization.

120. The polynucleotide according to any one of embodiments 65 to 119, wherein the dimerization unit comprises hinge exon h1 and hinge exon h4, a dimerization unit linker and a CH3 domain of human IgG3.

121. The polynucleotide according to embodiment 120, wherein the dimerization unit comprises or consists of an amino acid sequence having at least 80% sequence identity to the amino acid sequence 94 to 237 of SEQ ID NO: 1, such as at least 85%, or at least 86%, 87%, 88%, 89%, 90%, 91%, 92%, 93%, 94%, 95%, 96%, 97%, 98%, at least 99% or 100% sequence identity.

122. The polynucleotide according to any one of embodiments 65 to 121, wherein the polynucleotide is an RNA or DNA, preferably a DNA.

123. The polynucleotide according to any one of embodiments 65 to 122, wherein the polynucleotide further comprises a nucleotide sequence encoding a signal peptide.

124. The polynucleotide according to embodiment 123, wherein the signal peptide is selected from the list consisting of Ig VH signal peptide, human TPA signal peptide and human MIP-1α signal peptide.

125. The polynucleotide according to embodiment 124, wherein the signal peptide comprises or consists of an amino acid sequence having at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the amino acid sequence 1-23 of SEQ ID NO: 1.

126. The polynucleotide according to embodiment 125, wherein the targeting unit is human MIP-1α.

127. The polynucleotide according to embodiment 126, wherein the signal peptide comprises or consists of an amino acid sequence having at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the amino acid sequence 1-19 of SEQ ID NO: 2.

128. A vector comprising the polynucleotide according to any one of embodiments 65 to 127.

129. A host cell comprising the polynucleotide according to any one of embodiments 65 to 127 or the vector according to embodiment 128.

130. A polypeptide encoded by the nucleotide sequence as defined in any one of embodiments 1 to 64 or by the polynucleotide according to any one of embodiments 65 to 127.

131. A multimeric protein consisting of multiple polypeptides according to embodiment 130.

132. The multimeric protein according to embodiment 131, which is a dimeric protein consisting of two polypeptides according to embodiment 130.

133. The dimeric protein according to embodiment 132, wherein the dimeric protein is a homodimeric protein.

134. The polypeptide or the multimeric or dimeric protein according to any one of embodiments 131 to 134, wherein said targeting unit, multimerization unit, such as a dimerization unit, and antigenic unit in said polypeptide or multimeric protein, are in the N-terminal to C-terminal order of targeting unit, multimerization unit and antigenic unit.

135. The construct according to any one of embodiments 1 to 64, the polynucleotide according to any one of embodiments 65 to 127, the vector according to embodiment 128, the polypeptide according to embodiment 130, or the multimeric protein, such as the dimeric protein, according to any one of embodiments 130 to 132, for use as a medicament.

136. A vaccine comprising the construct according to any one of embodiments 1 to 64 or the polynucleotide according to any one of embodiments 65 to 127, and a pharmaceutically acceptable carrier.

137. The vaccine according to embodiment 136, wherein the pharmaceutically acceptable carrier is selected from the group consisting of saline, buffered saline, PBS, dextrose, water, glycerol, ethanol, sterile isotonic aqueous buffers, and combinations thereof.

138. The construct according to any one of embodiments 1 to 64, the polynucleotide according to any one of embodiments 65 to 127, or the vaccine according to any one of embodiments 136 to 137, for use in the treatment of an infectious disease caused by a pathogen.

139. The construct, polynucleotide or vaccine for the use according to embodiment 138, wherein the pathogen is selected from the group consisting of viruses, bacteria, fungi and parasites.

140. The construct, polynucleotide or vaccine for the use according to any one of embodiments 138 to 139, wherein the treatment is a prophylactic treatment or a therapeutic treatment.

141. The construct, polynucleotide or vaccine for the use according to any one of embodiments 138 to 140, wherein the treatment is a treatment of an infectious disease caused by the pathogen.

142. A method for preparing the vaccine according to any one of embodiments 136 to 137, wherein the vaccine comprises the polypeptide according to embodiment 130 or the multimeric protein, such as the dimeric protein, according to any one of embodiments 130 to 132,
wherein the method comprises:
a) transfecting cells with the polynucleotide according to any one of embodiments 65 to 125;
b) culturing the cells;
c) collecting and purifying the multimeric protein, such as the dimeric protein, or the polypeptide expressed from the cells; and
d) mixing the multimeric protein or the polypeptide obtained from step c) with a pharmaceutically acceptable carrier.

143. A method for preparing the vaccine according to any one of embodiments 136 to 137, wherein the vaccine comprises the polynucleotide according to embodiment 65, wherein the method comprises:
a) preparing the polynucleotide;
b) optionally cloning the polynucleotide into an expression vector; and
c) mixing the polynucleotide obtained from step a) or the vector obtained from step b) with the pharmaceutically acceptable carrier.

144. A method for treating a subject suffering from an infectious disease or being in need of prevention thereof, the method comprising administering to the subject the vaccine as defined in any one of embodiments 136 to 137.

145. The method according to embodiment 144, wherein the infectious disease is caused by a pathogen.

146. The method according to any one of embodiments 144 to 145, wherein the pathogen is selected from the group consisting of viruses, bacteria, fungi and parasites.

147. The method according to any one of embodiments 144 to 146, wherein the one or more T cell epitopes are from a pathogen, such a from a virus or a pathogenic bacteria.

148. The method according to any one of embodiments 144 to 146, wherein the one or more antigens are from a pathogen, such a from a virus or a pathogenic bacteria.

149. The method according to any one of embodiments 144 to 147, wherein the construct comprises a dimerization unit.

150. The method according to any one of embodiments 144 to 149, wherein the construct comprises a multimerization unit.

151. The method according to any one of embodiments 144 to 150, wherein the one or more antigen is a full-length protein of a pathogen, preferably a full-length surface protein of a pathogen.

152. The method according to any one of embodiments 144 to 151, wherein the one or more antigen is a full-length viral protein, preferably a full-length viral envelope protein.

153. The method according to any one of embodiments 144 to 147, wherein the one or more antigen is a full-length bacterial protein, preferably a full-length bacterial protein that is secreted or surface-localized.

154. The method according to any one of embodiments 144 to 153, wherein the antigenic unit comprises one full-length protein of a pathogen, preferably one full-length surface protein of a pathogen.

155. The method according to any one of embodiments 144 to 154, wherein the antigenic unit comprises several full-length proteins of a pathogen, preferably several full-length surface proteins of a pathogen.

156. The method according to any one of embodiments 144 to 155, wherein the antigenic unit comprises one or more parts or fragments of one or more antigens.

157. The method according to embodiment 156, wherein the antigenic unit comprises one part or fragment of one antigen.

158. The method according to embodiment 156, wherein the antigenic unit comprises several parts or fragments of one antigen.

159. The method according to embodiment 156, wherein the antigenic unit comprises one part or fragment of several antigens.

160. The method according to embodiment 156, wherein the antigenic unit comprises several parts or fragments of several antigens.

161. The method according to any one of embodiments 144 to 160, wherein the antigen is a surface protein of a pathogen.

162. The method according to embodiment 161, wherein the antigen is a viral surface protein, preferably a viral envelope protein.

163. The method according to any one of embodiments 144 to 162, wherein the antigen is a bacterial protein, preferably a bacterial protein that is secreted or surface-localized.

164. The method according to any one of embodiments 144 to 163, wherein the antigenic unit comprises multiple copies of antigens or parts or fragments thereof.

165. The method according to embodiment 164, wherein the antigenic unit comprises from 2 to 5 copies of antigens or parts or fragments thereof.

166. The method according to any one of embodiments 144 to 165, wherein the antigen or parts or fragments thereof comprises at least one B cell epitope, preferably several B cell epitopes.

167. The method according to any one of embodiments 144 to 166, wherein the subunit comprises n−1 T cell epitope linkers, wherein n is the number of neoepitopes in said antigenic unit and n is an integer of from 1 to 50.

168. The method according to any one of embodiments 144 to 167, wherein the subunit comprises 1 to 50 T cell epitopes.

169. The method according to embodiment 168, wherein the subunit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes or comprises 1 to 3 T cell epitopes or 1 to 5 T cell epitopes or 3 to 6 T cell epitopes or 5 to 15 T cell epitopes or 7 to 17 T cell epitopes or 9 to 19 T cell epitopes.

170. The method according to any one of embodiments 144 to 169, wherein the subunit comprises one T cell epitope.

171. The method according to any one of embodiments 144 to 170, wherein the subunit comprises several T cell epitopes.

172. The method according to any one of embodiments 144 to 171, wherein the T cell epitope is known to be immunogenic or is selected based on its predicted 30 ability to bind to HLA class I/II alleles.

173. The method according to any one of embodiments 144 to 172, wherein one or more of the T cell epitopes are known to be immunogenic and/or one or more of the T cell epitopes are selected based on the predicted ability to bind to HLA class I/II alleles.

174. The method according to any one of embodiments 144 to 173, wherein the T cell epitope is comprised in a structural protein or in a non-structural protein.

175. The method according to any one of embodiments 144 to 174, wherein the T cell epitope is from a conserved region of the pathogen.

176. The method according to any one of embodiments 144 to 175, wherein the T cell epitope has a length of from 7 to about 200 amino acids, such as from 7 to 100 amino acids, such as from 7 to 150 amino acids, preferably of from 7 to 100 amino acids, e.g. from about 10 to about 100 amino acids or from about 15 to about 100 amino acids or from about 20 to about 75 amino acids or from about 25 to about 50 amino acids; or the T cell epitope has a length suitable for presentation by HLA class I/II alleles, preferably a length of from 7 to 30 amino acids, more preferably a length of from 8 to 15 amino acids.

177. The method according to any one of embodiments 144 to 176, wherein the antigenic unit comprises up to 3500 amino acids, such as from 60 to 3500 amino acids, e.g. from about 80 or about 100 or about 150 amino acids to about a 3000 amino acids, such as from about 200 to about 2500 amino acids, such as from about 300 to about 2000 amino acids or from about 400 to about 1500 amino acids or from about 500 to about 1000 amino acids.

178. The method according to any one of embodiments 144 to 177, wherein the subunit comprises more than one T cell epitope, and the T cell epitope linker is a peptide consisting of from 4 to 40 amino acids, such as from 4 to 20 amino acids, such as from 5 to 20 amino acids or 5 to 15 amino acids or 8 to 20 amino acids or 8 to 15 amino acids 10 to 15 amino acids or 8 to 12 amino acids; preferably the T cell epitope linker is a peptide consisting of 10 amino acids.

179. The method according to any one of embodiments 144 to 178, wherein the antigen linker is a peptide consisting of from 10 to 80 amino acids, e.g. from 11 to 70 amino acids or 15 to 60 amino acids or 20 to 50 amino acids or 25 to 45 amino acids or 12 to 45 amino acids or 13 to 40 amino acids or 30 to 40 amino acids.

180. The method according to any one of embodiments 144 to 179, wherein the antigenic unit comprises several antigens or several parts of one or more antigens, and said several antigens or several parts of one or more antigens are separated from each other by an antigen linker as defined in embodiment 34.

181. The method according to any one of embodiments 144 to 180, wherein the T cell epitope linker and the antigen linker are more non-immunogenic and/or flexible, preferably non-immunogenic and flexible.

182. The method according to any one of embodiments 144 to 181, wherein the T cell epitope linker, if present, and the antigen linker are selected from the group consisting of: serine and/or glycine rich linkers which optionally comprise at least one leucine residue; GSAT linkers; and SEG linkers.

183. The method according to any one of embodiments 144 to 182, wherein the T cell epitope linker, if present, and the antigen linker are selected from the group consisting of serine and/or glycine rich linkers which optionally comprise at least one leucine residue, TQKSLSLSPGKGLGGL (SEQ ID NO: 78), SLSLSPGKGLGGL (SEQ ID NO: 79), GSAT linkers such as GGSAGGSGSGSSGGSSGASGTGTAGGTGSGSGTGSG (SEQ ID NO: 80) and SEG linkers such as GGSGGGSEGGGSEGGGSEGGGSEGGGSEGGGSGGGS (SEQ ID NO: 81).

184. The method according to any one of embodiments 144 to 183, wherein said targeting unit comprises antibody binding regions with specificity for surface molecules or receptors on antigen presenting cells (APCs), preferably wherein the surface molecule or receptor on APC is selected from the group consisting of HLA, CD14, CD40, CLEC9A, chemokine receptors, such as CCR1, CCR3, CCR4, CCR5, CCR6, CCR7, CCR8 or XCR1, and Toll-like receptors such as TLR-2, TLR-4 or TLR-5.

185. The method according to any one of embodiments 144 to 184, wherein the targeting unit has affinity for a chemokine receptor selected from CCR1, CCR3 and CCR5.

186. The method according to any one of embodiments 144 to 185, wherein the targeting unit has affinity for MHC class II proteins, preferably MHC class II proteins selected from the group consisting of anti-HLA-DP, anti-HLA-DR and anti-pan HLA class II.

187. The method according to any one of embodiments 144 to 186, wherein the targeting unit comprises or consists of pan-HLAII or MIP-1α, preferably pan-HLAII or human MIP-1α (LD78β, CCL3L1).

188. The method according to embodiment 187, wherein the targeting unit is MIP-1α, preferably human MIP-1α.

189. The method according to embodiment 188, wherein the targeting unit comprises or consists of an amino acid sequence having at least 80% sequence identity to the amino acid sequence 24-93 of SEQ ID NO: 1, such as at least 85% or at least 86%, 87%, 88%, 89%, 90%, 91%, 92%, 93%, 94%, 95%, 96%, 97%, 98%, at least 99% or 100% sequence identity.

190. The method according to any one of embodiments 144 to 187, wherein the targeting unit is anti-pan-HLA class II.

191. The method according to embodiment 190, wherein the targeting unit comprises an amino acid sequence having at least 80% sequence identity to the amino acid sequence 20-260 of SEQ ID NO: 2, such as at least 85% or at least 86%, 87%, 88%, 89%, 90%, 91%, 92%, 93%, 94%, 95%, 96%, 97%, 98%, at least 99% or 100% sequence identity.

192. The method according to any one of embodiments 144 to 191, wherein the multimerization unit is a dimerization unit which comprises a hinge region, such as hinge exon h1 and hinge exon h4.

193. The method according to embodiment 192, wherein the hinge region has the ability to form one or more covalent bonds.

194. The method according to any one of embodiments 192 to 193, wherein the hinge region is Ig derived.

195. The method according to any one of embodiments 192 to 194, wherein the dimerization unit further comprises another domain that facilitates dimerization.

196. The method according to embodiment 195, wherein the other domain is an immunoglobulin domain, preferably an immunoglobulin constant domain.

197. The method according to any one of embodiments 195 to 196, wherein the other domain is a carboxyterminal C domain derived from IgG, preferably from IgG3.

198. The method according to any one of embodiments 192 to 197, wherein the dimerization unit further comprises a dimerization unit linker, such as glycine-serine rich linker, such as GGGSSGGGSG (SEQ ID NO: 87).

199. The method according to embodiment 198, wherein the dimerization unit linker connects the hinge region and the other domain that facilitates dimerization.

200. The method according to any of embodiments 192 to 199, wherein the dimerization unit comprises hinge exon h1 and hinge exon h4, a dimerization unit linker and a CH3 domain of human IgG3.

201. The method according to embodiment 200, wherein the dimerization unit comprises or consists of an amino acid sequence having at least 80% sequence identity to the amino acid sequence 94 to 237 of SEQ ID NO: 1, such as at least 85%, or at least 86%, 87%, 88%, 89%, 90%, 91%, 92%, 93%, 94%, 95%, 96%, 97%, 98%, at least 99% or 100% sequence identity.

202. The method according to any one of embodiments 144 to 201, wherein the construct is the polynucleotide (i).

203. The method according to embodiment 202, wherein the polynucleotide is an RNA or DNA, preferably a DNA.

204. The method according to any one of embodiments 144 to 203, wherein the polynucleotide further comprises a nucleotide sequence encoding a signal peptide.

205. The method according to embodiment 204, wherein the signal peptide is selected from the list consisting of Ig VH signal peptide, human TPA signal peptide and human MIP-1α signal peptide.

206. The method according to embodiment 205, wherein the signal peptide comprises or consists of an amino acid sequence having at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the amino acid sequence 1-23 of SEQ ID NO: 1.

207. The method according to embodiment 206, wherein the targeting unit is human MIP-1α.

208. The method according to embodiment 205, wherein the signal peptide comprises or consists of an amino acid sequence having at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the amino acid sequence 1-19 of SEQ ID NO: 2.

209. The method according to embodiment 208, wherein the targeting unit is anti-pan HLA class II.

210. A vaccine as defined in any one of embodiments 136 to 137 for use in the treatment of an infectious disease.

211. The vaccine according to embodiment 147, wherein the infectious disease is caused by a pathogen.

212. The vaccine according to any one of embodiments 147 to 211, wherein 15 the pathogen is selected from the group consisting of viruses, bacteria, fungi and parasites.

213. The vaccine according to any of one of embodiments 147 to 212, wherein the treatment is a prophylactic treatment or a therapeutic treatment.

214. A kit comprising the construct according to any one of embodiments 1 to 64, the polynucleotide according to any one of embodiments 65 to 127, the vaccine according to any one of embodiments 210 to 213, or the polypeptide according to embodiment 130 or the multimeric protein, such as the dimeric protein, according to any one of embodiments 130 to 132, and optionally instructions for use.

1. An immunogenic construct comprising:
(i) a polynucleotide comprising a nucleotide sequence encoding a polypeptide comprising:
a) a targeting unit targeting or capable of targeting antigen-presenting cells,
b) a multimerization unit, such as a dimerization unit; and
c) an antigenic unit comprising one or more T cell epitopes and one or more antigens or parts or fragments thereof; or
(ii) a polypeptide encoded by the nucleotide sequence defined in (i); or
(iii) a multimeric protein consisting of multiple polypeptides as defined in (ii), such as a dimeric protein consisting of two polypeptides as defined in (ii);
wherein the one or more T cell epitopes and the one or more antigens or parts or fragments thereof are from a pathogen;
wherein the antigenic unit comprises a subunit comprising the T cell epitopes which are separated from each other by T cell epitope linkers, if more than one T cell epitope is comprised in the subunit,
and wherein the subunit is connected to the multimerization unit by a unit linker and separated from the one or more antigens or parts or fragments thereof by an antigen linker.

2. The construct according to claim 1, wherein the targeting unit comprises a moiety that interacts with surface molecules on the antigen-presenting cells, such as a surface molecule selected from the group consisting of HLA, CD14, CD40, CLEC9A, chemokine receptors, such as CCR1, CCR3, CCR4, CCR5, CCR6, CCR7, CCR8 or XCR1 and Toll-like receptors such as TLR-2, TLR-4 or TLR-5.

3. The construct according to claim 1, wherein the targeting unit comprises or consists of soluble CD40 ligand, CCL4 and its isoforms, CCL5, CCL19, CCL20, CCL21, macrophage inflammatory protein alpha including its isoforms, such as mouse CCL3, human CCL3, human CCL3L1, human CCL3L2 and human CCL3L3, XCL1, XCL2, flagellin, anti-HLA-DP, anti-HLA-DR, anti-pan HLA class II anti-CD40, anti-TLR-2, anti-TLR-4, anti-TLR-5 or anti-CLEC9A.

4. The construct according to claim 1, wherein the multimerization unit is a trimerization unit, such as the C-terminal domain of T4 fibritin or such as a collagen-derived trimerization unit, such as human collagen derived XVIII trimerization domain or human collagen XV trimerization domain, or a tetramerization unit, such as a domain derived from p53.

5. The construct according to claim 1, wherein the multimerization unit is a dimerization unit, such as a dimerization unit comprising a hinge region and an immunoglobulin domain, e.g. an immunoglobulin constant domain or a dimerization unit comprising the dHLX protein.

6. The construct according to claim 1, wherein the construct further comprises a unit liker that connects the antigenic unit to the multimerization unit, such as the dimerization unit, and wherein the unit linker is a non-immunogenic linker and/or flexible or rigid linker.

7. The construct according to claim 1, wherein the construct is a polynucleotide, such as an RNA or DNA.

8. The construct according to claim 7, wherein the polynucleotide further comprises a nucleotide sequence encoding a signal peptide.

9. The construct according to claim 1, wherein the construct comprises a DNA polynucleotide comprising a nucleotide sequence encoding a polypeptide comprising a human MIP-1α signal peptide, a human MIP-1α targeting unit, a dimerization unit which comprises a hinge exon h1, a hinge exon h4, a dimerization unit linker and a CH3 domain of human IgG3, a unit linker and an antigenic unit as defined in claim 1.

10. (canceled)

11. A vaccine comprising the construct according to claim 1, and a pharmaceutically acceptable carrier.

12-19. (canceled)

20. The vaccine according to claim 11, wherein the vaccine is a liquid formulation for injection.

21. A method for preparing the vaccine according to claim 11, wherein the vaccine comprises the polypeptide or the multimeric protein,
wherein the method comprises:
a) transfecting cells with the polynucleotide;
b) culturing the cells;
c) collecting and purifying the multimeric protein, such as the dimeric protein, or the polypeptide expressed from the cells; and
d) mixing the multimeric protein or the polypeptide obtained from step c) with a pharmaceutically acceptable carrier.

22. A method for treating a subject suffering from a disease caused by a pathogen or being in need of prevention thereof, wherein the method comprises administering to the subject the vaccine as defined in claim 11.

23-31. (canceled)

32. A vector comprising the construct of claim 7.

33. The construct according to claim 1, wherein the pathogen is selected from the group consisting of virus, bacterium, fungus and parasite.

34. A method for preparing the vaccine according to claim 11, wherein the vaccine comprises the polynucleotide, wherein the method comprises:
a) Preparing the polynucleotide;
b) Optionally cloning the polynucleotide into an expression vector; and
c) Mixing the polynucleotide obtained from step a) or the vector obtained from step b) with a pharmaceutically acceptable carrier.

35. The method of claim 22, wherein the pathogen is selected from the group consisting of virus, bacterium, fungus and parasite.
